(19) United States
(12) Patent Application Publication
(10) Pub. No.: US 2026/0229325 A1
(43) Pub. Date: Aug. 6, 2026

(54) ARTIFICIAL INTELLIGENCE SYSTEM FOR GENERATING PHARMACEUTICAL INTERVENTION PLANS BASED ON PATIENT PROFILES

(71) Applicant: Taura Health, Inc., Lake Geneva, WI (US)

(72) Inventors: Jeffrey Scott Shorten, Eden Prairie, MN (US); Justin Lee Heiser, Alexandria, MN (US)

(21) Appl. No.: 19/288,104

(22) Filed: Aug. 1, 2025

Related U.S. Application Data
(63) Continuation of application No. 17/698,734, filed on Mar. 18, 2022, now abandoned.
(60) Provisional application No. 63/163,247, filed on Mar. 19, 2021.

(51) Int. Cl. G16H 10/60; G16H 10/20; G16H 20/10; G16H 40/20; G16H 70/40; G16H 80/00

(52) U.S. Cl. CPC G16H 10/60; G16H 10/20; G16H 20/10; G16H 40/20; G16H 70/40; G16H 80/00

(57) ABSTRACT

Techniques for utilizing artificial intelligence to generate intervention plans based on pharmaceutical profiles are described. A computing device creates a pharmaceutical profile for a patient, the pharmaceutical profile including information regarding one or more of one or more active prescriptions for the patient, one or more inactive past prescriptions for the patient, one or more diagnosis codes for the patient, prescription directions for the one or more active prescriptions for the patient, allergy information for the patient, clinical interventions for the patient, insurance information for the patient, a preferred store for the patient, and demographic information for the patient. The computing device receives real-time prescription information for the patient and generates an intervention plan for the patient based on the pharmaceutical profile for the patient and the real-time prescription information for the patient.

### RELATED APPLICATIONS

This application is a continuation of U.S. patent application Ser. No. 17/698,734, filed Mar. 18, 2022, which claims the benefit of U.S. Provisional Patent Application No. 63/163,247, filed Mar. 19, 2021, the entire contents of which are incorporated herein.

### TECHNICAL FIELD

The disclosure relates to artificial intelligence and workflow optimization.

### BACKGROUND

Pharmaceutical workflows generally include receiving the patient and prescription information from a doctor. At the pharmacy at which the prescribed medication may be retrieved, a licensed pharmacist is required to review the prescription and patient information, verify the accuracy of all received information, configure the prescription, and fill the information. This can lead to bottlenecks at locations that are busier or understaffed, while other locations that may be more rural or properly staffed may not be as busy.

### SUMMARY

In one example, the disclosure is directed to a non-transitory computer-readable storage medium containing instructions. The instructions, when executed, cause one or more processors to receive, from a first computing device of a plurality of computing devices, prescription data for a first prescription to be filled, wherein the first computing device is located in a first geographic location that is different than a second geographic location where a second computing device of the plurality of computing devices is located, wherein the prescription data includes a promise time comprising a time at which the prescription is scheduled to be retrieved by a patient and prescription information to be validated by a pharmacist. The instructions, when executed, further cause the one or more processors to, in response to receiving the prescription data for the first prescription, determine, based on the promise time for the first prescription, a first position on a list of prescriptions for the first prescription, wherein the list of prescriptions comprises one or more prescriptions to be validated by a pharmacist, wherein the list of prescriptions is sorted by a respective promise time for each of the one or more prescriptions to be validated, and wherein each prescription in the one or more prescriptions to be validated was received by the server device and from one of the plurality of computing devices. The instructions, when executed, also cause the one or more processors to, place an indication of the first prescription in the first position on the list of prescriptions to create an updated list. The instructions, when executed, further cause the one or more processors to, in response to creating the updated list, send the updated list to each computing device of the plurality of computing devices.

In another example, the disclosure is directed to a non-transitory computer-readable storage medium containing instructions. The instructions, when executed, cause one or more processors to receive, from a server device, a list of prescriptions comprising one or more prescriptions to be validated by a pharmacist, wherein the list of prescriptions is sorted by at least a respective promise time for each of the one or more prescriptions to be validated, wherein each prescription in the one or more prescriptions to be validated was received by the server device from one of a plurality of computing devices, wherein the plurality of computing devices includes the first computing device, wherein the first computing device is located in a first geographic location, wherein a first prescription in the list of prescriptions was sent to the server device by a second computing device of the plurality of computing devices, and wherein the second computing device is located in a second geographic location different than the first geographic location. The instructions, when executed, further cause the one or more processors to output, for display on a display device operatively connected to the first computing device, at least a portion of the list of prescriptions in a first graphical user interface. The instructions, when executed, also cause the one or more processors to receive an indication of user input selecting the first prescription from the list of prescriptions in the graphical user interface. The instructions, when executed, further cause the one or more processors to, in response to receiving the indication of user input selecting the first prescription, output, for display on the display device operatively connected to the first computing device, prescription information to be validated for the first prescription in a second graphical user interface.

In another example, the disclosure is directed to a method comprising receiving, by a server device and from a first computing device of a plurality of computing devices, prescription data for a first prescription to be filled, wherein the first computing device is located in a first geographic location that is different than a second geographic location where a second computing device of the plurality of computing devices is located, wherein the prescription data includes a promise time comprising a time at which the prescription is scheduled to be retrieved by a patient and prescription information to be validated by a pharmacist. The method further includes, in response to receiving the prescription data for the first prescription, determining, by the server device and based on the promise time for the first prescription, a first position on a list of prescriptions for the first prescription, wherein the list of prescriptions comprises one or more prescriptions to be validated by a pharmacist, wherein the list of prescriptions is sorted by a respective promise time for each of the one or more prescriptions to be validated, and wherein each prescription in the one or more prescriptions to be validated was received by the server device and from one of the plurality of computing devices. The method also includes placing, by the server device, an indication of the first prescription in the first position on the list of prescriptions to create an updated list. The method further includes, in response to creating the updated list, sending, by the server device, the updated list to each computing device of the plurality of computing devices. The method also includes receiving, by a second computing device of the plurality of computing devices and from the server device, the updated list, wherein the second computing device is located in a second geographic location different than the first geographic location. The method further includes outputting, by the second computing device and for display on a display device operatively connected to the second computing device, at least a portion of the updated list in a first graphical user interface. The method also includes receiving, by the second computing device, an indication of user input selecting the first prescription from the list of prescriptions in the graphical user interface. The method further includes, in response to receiving the indication of user input selecting the first prescription, outputting, by the second computing device and for display on the display device operatively connected to the second computing device, prescription information to be validated for the first prescription in a second graphical user interface.

In another example, the disclosure is directed to a non-transitory computer-readable storage medium containing instructions. The instructions, when executed, cause one or more processors to create a pharmaceutical profile for a patient, wherein the pharmaceutical profile comprises information regarding one or more of one or more active prescriptions for the patient, one or more inactive past prescriptions for the patient, one or more diagnosis codes for the patient, prescription directions for the one or more active prescriptions for the patient, allergy information for the patient, clinical interventions for the patient, insurance information for the patient, a preferred store for the patient, and demographic information for the patient. The instructions, when executed, further cause the one or more processors to receive real-time prescription information for the patient. The instructions, when executed, also cause the one or more processors to generate an intervention plan for the patient based on the pharmaceutical profile for the patient and the real-time prescription information for the patient.

In another example, the disclosure is directed to a method including creating, by one or more processors of a computing device, a pharmaceutical profile for a patient, wherein the pharmaceutical profile comprises information regarding one or more of one or more active prescriptions for the patient, one or more inactive past prescriptions for the patient, one or more diagnosis codes for the patient, prescription directions for the one or more active prescriptions for the patient, allergy information for the patient, clinical interventions for the patient, insurance information for the patient, a preferred store for the patient, and demographic information for the patient. The method further includes receiving, by the one or more processors, real-time prescription information for the patient. The method also includes generating, by the one or more processors, an intervention plan for the patient based on the pharmaceutical profile for the patient and the real-time prescription information for the patient.

In another example, the disclosure is directed to a computing device comprising a storage component and one or more processors. The one or more processors are configured to create a pharmaceutical profile for a patient, wherein the pharmaceutical profile comprises information regarding one or more of one or more active prescriptions for the patient, one or more inactive past prescriptions for the patient, one or more diagnosis codes for the patient, prescription directions for the one or more active prescriptions for the patient, allergy information for the patient, clinical interventions for the patient, insurance information for the patient, a preferred store for the patient, and demographic information for the patient. The one or more processors are further configured to receive real-time prescription information for the patient. The one or more processors are also configured to generate an intervention plan for the patient based on the pharmaceutical profile for the patient and the real-time prescription information for the patient.

The details of one or more examples of the disclosure are set forth in the accompanying drawings and the description below. Other features, objects, and advantages of the disclosure will be apparent from the description and drawings, and from the claims.

### BRIEF DESCRIPTION OF DRAWINGS

The following drawings are illustrative of particular examples of the present invention and therefore do not limit the scope of the invention. The drawings are not necessarily to scale, though embodiments can include the scale illustrated, and are intended for use in conjunction with the explanations in the following detailed description wherein like reference characters denote like elements. Examples of the present invention will hereinafter be described in conjunction with the appended drawings.

FIG. 1 is a block diagram illustrating an example system of pharmacies at separate locations and with respective client devices connected over a network, in accordance with one or more of the techniques described herein.

FIG. 2 is a block diagram illustrating a more detailed example of a server device configured to perform the techniques described herein.

FIG. 3 is a block diagram illustrating a more detailed example of a client device configured to perform the techniques described herein.

FIG. 4 is a flow diagram illustrating an example technique performed by one or more devices within the system described herein.

FIG. 5 is an example portion of a graphical user interface showing an example prescription fill list, in accordance with one or more of the techniques described herein.

FIG. 6 is an example portion of a graphical user interface showing an example prescription fill list with a long-term care option, in accordance with one or more of the techniques described herein.

FIG. 7 is an example portion of a graphical user interface showing an example image prescription verification page, in accordance with one or more of the techniques described herein.

FIG. 8 is an example portion of a graphical user interface showing an example text prescription verification page, in accordance with one or more of the techniques described herein.

FIG. 9 is an example portion of a graphical user interface showing an example prescription image with a consultation notes field, in accordance with one or more of the techniques described herein.

FIG. 10 is an example portion of a graphical user interface showing an example prescription entry page, in accordance with one or more of the techniques described herein.

FIG. 11 is an example portion of a graphical user interface showing an example button to remove consultation notes, in accordance with one or more of the techniques described herein.

FIG. 12 is an example portion of a graphical user interface showing an example biometric verification request, in accordance with one or more of the techniques described herein.

FIG. 13 is an example portion of a graphical user interface showing an example prescription with a clinical hold and a biometric verification request, in accordance with one or more of the techniques described herein.

FIG. 14 is an example portion of a graphical user interface showing an example dialog box to change the promised prescription time, in accordance with one or more of the techniques described herein.

FIG. 15 is an example portion of a graphical user interface showing an example page to verify a prescription for a controlled substance, in accordance with one or more of the techniques described herein.

FIG. 16 is an example portion of a graphical user interface showing an example fill verify queue, in accordance with one or more of the techniques described herein.

FIG. 17 is an example portion of a graphical user interface showing an example prescription pill image, in accordance with one or more of the techniques described herein.

FIG. 18 is an example portion of a graphical user interface showing an example denial of a prescription, in accordance with one or more of the techniques described herein.

FIG. 19 is an example portion of a graphical user interface showing an example blister packet verification with both full and half tablets, in accordance with one or more of the techniques described herein.

FIG. 20 is an example portion of a graphical user interface showing an example prescription fill list for long term care admissions, in accordance with one or more of the techniques described herein.

FIG. 21 is an example portion of a graphical user interface showing an example prescription fill list for long term care admissions with multiple prescriptions in a single order, in accordance with one or more of the techniques described herein.

FIG. 22 is an example portion of a graphical user interface showing an example search page, in accordance with one or more of the techniques described herein.

FIG. 23 is an example portion of a graphical user interface showing an example patient workflow history, in accordance with one or more of the techniques described herein.

FIG. 24 is an example portion of a graphical user interface showing an example specialty prescription list, in accordance with one or more of the techniques described herein.

FIG. 25 is an example portion of a graphical user interface showing an example quality assurance queue, in accordance with one or more of the techniques described herein.

FIG. 26 is an example portion of a graphical user interface showing an example clinical queue, in accordance with one or more of the techniques described herein.

FIG. 27 is an example portion of a graphical user interface showing an example clinical conversation page, in accordance with one or more of the techniques described herein.

FIG. 28 is an example portion of a graphical user interface showing an example prescription fill queue with multiple prescriptions being locked, in accordance with one or more of the techniques described herein.

FIG. 29 is an example portion of a graphical user interface showing an example prescription list with a tab to access a clinical platform, in accordance with one or more of the techniques described herein.

FIG. 30 is an example portion of a graphical user interface showing an example clinical platform, in accordance with one or more of the techniques described herein.

FIG. 31 is an example portion of a graphical user interface showing an example of information usable during patient conversations, in accordance with one or more of the techniques described herein.

FIG. 32 is an example portion of a graphical user interface showing an example allergy information page, in accordance with one or more of the techniques described herein.

FIG. 33 is an example portion of a graphical user interface showing an example patient profile, in accordance with one or more of the techniques described herein.

FIG. 34 is an example portion of a graphical user interface showing an example patient profile with inactive medications, in accordance with one or more of the techniques described herein.

FIG. 35 is an example portion of a graphical user interface showing an example list of medications filled by third party pharmacists, in accordance with one or more of the techniques described herein.

FIG. 36 is an example portion of a graphical user interface showing an example primary care provider page, in accordance with one or more of the techniques described herein.

FIG. 37 is an example portion of a graphical user interface showing an example patient demographic page with expandable tiles, in accordance with one or more of the techniques described herein.

FIG. 38 is an example portion of a graphical user interface showing an example patient profile with substitute recommendations, in accordance with one or more of the techniques described herein.

FIG. 39 is an example portion of a graphical user interface showing an example billing information page, in accordance with one or more of the techniques described herein.

FIG. 40 is an example portion of a graphical user interface showing an example billing information page with a deferred recommendation, in accordance with one or more of the techniques described herein.

FIG. 41 is an example portion of a graphical user interface showing an example new medication recommendation, in accordance with one or more of the techniques described herein.

FIG. 42 is an example portion of a graphical user interface showing an example printing dialog box, in accordance with one or more of the techniques described herein.

FIG. 43 is an example portion of a graphical user interface showing an example clinical history page, in accordance with one or more of the techniques described herein.

FIG. 44 is an example portion of a graphical user interface showing an example recommendation history page, in accordance with one or more of the techniques described herein.

FIG. 45 is an example portion of a graphical user interface showing an example recommendation direction page, in accordance with one or more of the techniques described herein.

FIG. 46 is an example portion of a graphical user interface showing an example contact log, in accordance with one or more of the techniques described herein.

FIG. 47 is an example portion of a graphical user interface showing an example clinical document list, in accordance with one or more of the techniques described herein.

FIG. 48 is an example portion of a graphical user interface showing an example option to add a program, in accordance with one or more of the techniques described herein.

FIG. 49 is an example portion of a graphical user interface showing an example prompt for adding a program, in accordance with one or more of the techniques described herein.

FIG. 50 is an example portion of a graphical user interface showing an example contact method selection, in accordance with one or more of the techniques described herein.

FIG. 51 is an example portion of a graphical user interface showing an example list of test cases and targeted medication reviews (TMR), in accordance with one or more of the techniques described herein.

FIG. 52 is an example portion of a graphical user interface showing an example targeted intervention list, in accordance with one or more of the techniques described herein.

FIG. 53 is an example portion of a graphical user interface showing an example option to add a comprehensive medical review test case, in accordance with one or more of the techniques described herein.

FIG. 54 is an example portion of a graphical user interface showing an example prompt to add a test case, in accordance with one or more of the techniques described herein.

FIG. 55 is an example portion of a graphical user interface showing an example test case recommendation entry page, in accordance with one or more of the techniques described herein.

FIG. 56 is an example portion of a graphical user interface showing an example test case page with an option to add a TMR, in accordance with one or more of the techniques described herein.

FIG. 57 is an example portion of a graphical user interface showing an example prompt to add a TMR, in accordance with one or more of the techniques described herein.

FIG. 58 is an example portion of a graphical user interface showing an example prompt to add a test case condition, in accordance with one or more of the techniques described herein.

FIG. 59 is an example portion of a graphical user interface showing an example response group option, in accordance with one or more of the techniques described herein.

FIG. 60 is an example portion of a graphical user interface showing an example prompt for editing a response group, in accordance with one or more of the techniques described herein.

### DETAILED DESCRIPTION

The following detailed description is exemplary in nature and is not intended to limit the scope, applicability, or configuration of the invention in any way. Rather, the following description provides some practical illustrations for implementing examples of the present invention. Those skilled in the art will recognize that many of the noted examples have a variety of suitable alternatives.

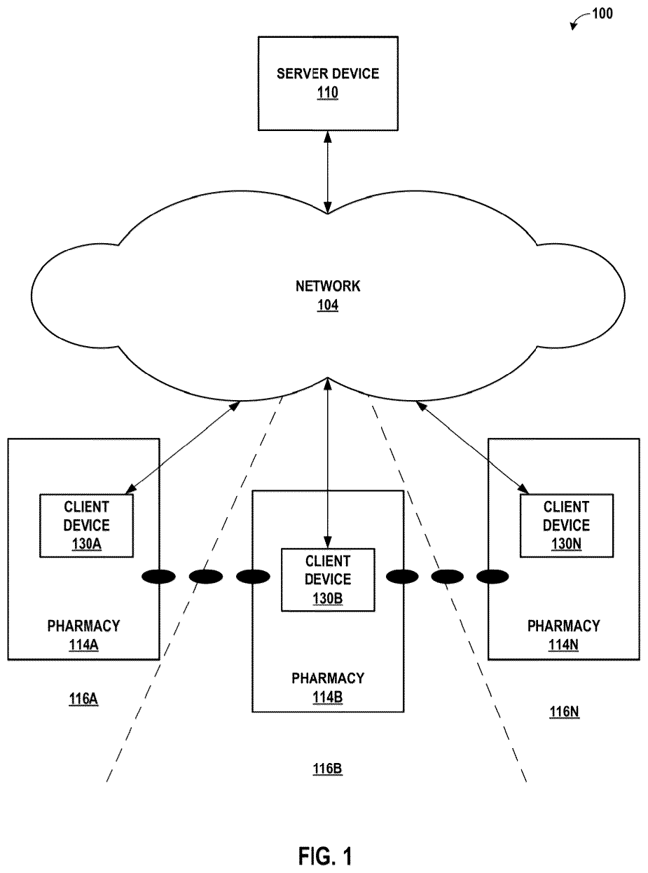

FIG. 1 is a block diagram illustrating an example system 100 of pharmacies 114A-114N at separate locations 116A-116N and with respective client devices 130A-130N connected over a network 104, in accordance with one or more of the techniques described herein. Server device 110 may be a central server device, such as one located at a corporate headquarters when pharmacies 114A-114N are affiliated pharmacies, or at some central repository located at one of pharmacies 114A-114N.

In accordance with one or more techniques described herein, server device 110 may receive, from client device 130A, prescription data for a first prescription to be filled. Client device 130A is located in geographic location 116A that is different than a geographic location 116B where client device 130B is located. The prescription data includes a promise time, or a time at which the prescription is scheduled to be retrieved by a patient and prescription information to be validated by a pharmacist.

In response to receiving the prescription data for the first prescription, server device 110 may determine, based on the promise time for the first prescription, a first position on a list of prescriptions for the first prescription. The list of prescriptions may include one or more prescriptions to be validated by a pharmacist, and the list of prescriptions may be sorted by a respective promise time for each of the one or more prescriptions to be validated. Each prescription in the one or more prescriptions to be validated was received by server device 110 and from one of client devices 130A-130N.

Server device 110 may subsequently place an indication of the first prescription in the first position on the list of prescriptions to create an updated list. In response to creating the updated list, server device 110 may send the updated list to each of client devices 130A-130N. As such, client devices 130A-130N may each have a combined copy of prescriptions from each of pharmacies 114A-114N to create a unified, singular workflow that each client device 130A-130N has access to.

In accordance with the techniques described herein, client device 130A may receive, from server device 110, a list of prescriptions that includes one or more prescriptions to be validated by a pharmacist. The list of prescriptions is sorted by at least a respective promise time for each of the one or more prescriptions to be validated, with each prescription in the one or more prescriptions to be validated having been received by server device 110 from one of client devices 130A-130N. Client device 130A is located in geographic location 116A. A first prescription in the list of prescriptions was sent to server device 110 by client device 130B, and client device 130B is located in geographic location 116B different than geographic location 116A.

Client device 130A may output, for display on a display device operatively connected to client device 130A, at least a portion of the list of prescriptions in a first graphical user interface. Client device 130A may then receive an indication of user input selecting the first prescription from the list of prescriptions in the graphical user interface, the first prescription being the prescription sent by client device 130B for pickup at location 116B and pharmacy 114B, even though client device 130A is at different location 116A and pharmacy 114A. In response to receiving the indication of user input selecting the first prescription, client device 130A may output, for display on the display device operatively connected to client device 130A, prescription information to be validated for the first prescription in a second graphical user interface.

In accordance with additional techniques described herein, server device 110 may create a pharmaceutical profile for a patient. The pharmaceutical profile may include information regarding one or more of one or more active prescriptions for the patient, one or more inactive past prescriptions for the patient, one or more diagnosis codes for the patient, prescription directions for the one or more active prescriptions for the patient, allergy information for the patient, clinical interventions for the patient, insurance information for the patient, a preferred store for the patient, and demographic information for the patient. Server device 110 may then receive real-time prescription information for the patient and generate an intervention plan for the patient based on the pharmaceutical profile for the patient and the real-time prescription information for the patient.

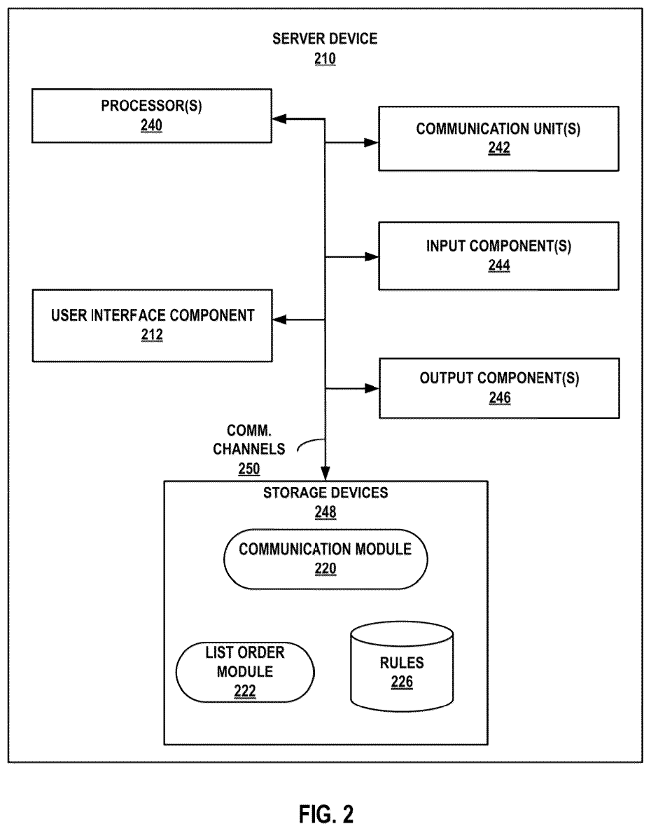

FIG. 2 is a block diagram illustrating a more detailed example of a server device configured to perform the techniques described herein. Server device 210 of FIG. 2 is described below as an example of server device 110 of FIG. 1. FIG. 2 illustrates only one particular example of server device 210, and many other examples of server device 210 may be used in other instances and may include a subset of the components included in example server device 210 or may include additional components not shown in FIG. 2.

As shown in the example of FIG. 2, server device 210 includes user interface component (UIC) 212, one or more processors 240, one or more communication units 242, one or more input components 244, one or more output components 246, and one or more storage components 248. Storage components 248 of server device 210 include communication module 220, list order module 222, and rules data store 226.

One or more processors 240 may implement functionality and/or execute instructions associated with server device 210 to control a prescription list that is verified by numerous remote client devices. That is, processors 240 may implement functionality and/or execute instructions associated with server device 210 to compile prescriptions from a number of client devices and take prescriptions off the list as those remote client devices verify the prescriptions.

Examples of processors 240 include application processors, display controllers, auxiliary processors, one or more sensor hubs, and any other hardware configure to function as a processor, a processing unit, or a processing device. Modules 220 and 222 may be operable by processors 240 to perform various actions, operations, or functions of server device 210. For example, processors 240 of server device 210 may retrieve and execute instructions stored by storage components 248 that cause processors 240 to perform the operations described with respect to modules 220 and 222.

One or more storage components 248 within server device 210 may store information for processing during operation of server device 210 (e.g., server device 210 may store data accessed by modules 220 and 222 and rules data store 226 during execution at server device 210). In some examples, storage component 248 is a temporary memory, meaning that a primary purpose of storage component 248 is not long-term storage. Storage components 248 on server device 210 may be configured for short-term storage of information as volatile memory and therefore not retain stored contents if powered off. Examples of volatile memories include random access memories (RAM), dynamic random access memories (DRAM), static random access memories (SRAM), and other forms of volatile memories known in the art.

Storage components 248, in some examples, also include one or more computer-readable storage media. Storage components 248 in some examples include one or more non-transitory computer-readable storage mediums. Storage components 248 may be configured to store larger amounts of information than typically stored by volatile memory. Storage components 248 may further be configured for long-term storage of information as non-volatile memory space and retain information after power on/off cycles. Examples of non-volatile memories include magnetic hard discs, optical discs, floppy discs, flash memories, or forms of electrically programmable memories (EPROM) or electrically erasable and programmable (EEPROM) memories. Storage components 248 may store program instructions and/or information (e.g., data) associated with modules 220 and 222 and rules data store 226. Storage components 248 may include a memory configured to store data or other information associated with modules 220 and 222 and rules data store 226.

Communication channels 250 may interconnect each of the components 212, 240, 242, 244, 246, and 248 for inter-component communications (physically, communicatively, and/or operatively). In some examples, communication channels 250 may include a system bus, a network connection, an inter-process communication data structure, or any other method for communicating data.

One or more communication units 242 of server device 210 may communicate with external devices via one or more wired and/or wireless networks by transmitting and/or receiving network signals on one or more networks. Examples of communication units 242 include a network interface card (e.g. such as an Ethernet card), an optical transceiver, a radio frequency transceiver, a GPS receiver, or any other type of device that can send and/or receive information. Other examples of communication units 242 may include short wave radios, cellular data radios, wireless network radios, as well as universal serial bus (USB) controllers.

One or more input components 244 of server device 210 may receive input. Examples of input are tactile, audio, and video input. Input components 244 of server device 210, in one example, includes a presence-sensitive input device (e.g., a touch sensitive screen, a PSD), mouse, keyboard, voice responsive system, camera, microphone or any other type of device for detecting input from a human or machine.

One or more output components 246 of server device 210 may generate output in a selected modality. Examples of modalities may include a tactile notification, audible notification, visual notification, machine generated voice notification, or other modalities. Output components 246 of server device 210, in one example, includes a presence-sensitive display, sound card, video graphics adapter card, speaker, cathode ray tube (CRT) monitor, liquid crystal display (LCD), or any other type of device for generating output to a human or machine in a selected modality.

While illustrated as an internal component of server device 210, UIC 212 may also represent an external component that shares a data path with server device 210 for transmitting and/or receiving input and output. For instance, in one example, UIC 212 represents a built-in component of server device 210 located within and physically connected to the external packaging of server device 210 (e.g., a screen on a mobile phone). In another example, UIC 212 represents an external component of server device 210 located outside and physically separated from the packaging or housing of server device 210 (e.g., a monitor, a projector, etc. that shares a wired and/or wireless data path with server device 210).

UIC 212 of server device 210 may detect two-dimensional and/or three-dimensional gestures as input from a user of server device 210. For instance, a sensor of UIC 212 may detect a user's movement (e.g., moving a hand, an arm, a pen, a stylus, a tactile object, etc.) within a threshold distance of the sensor of UIC 212. UIC 212 may determine a two or three-dimensional vector representation of the movement and correlate the vector representation to a gesture input (e.g., a hand-wave, a pinch, a clap, a pen stroke, etc.) that has multiple dimensions. In other words, UIC 212 can detect a multi-dimension gesture without requiring the user to gesture at or near a screen or surface at which UIC 212 outputs information for display. Instead, UIC 212 can detect a multi-dimensional gesture performed at or near a sensor which may or may not be located near the screen or surface at which UIC 212 outputs information for display.

In accordance with the techniques described herein, communication module 220 may receive, from a first client device of a plurality of client devices, prescription data for a first prescription to be filled. The first client device may be located in a first geographic location that is different than a second geographic location where a second client device of the plurality of client devices is located. The prescription data includes a promise time, or a time at which the prescription is scheduled to be retrieved by a patient, and prescription information to be validated by a pharmacist. The prescription information may include one or more of a patient name, a prescriber name, a prescription type, a fill type, a patient date of birth, a drug name, a drug strength, a drug form, a quantity written, a day supply, a refill amount, a drug application instruction, a prescriber license, and a prescription date. The first prescription comprises one or more of an individual prescription for an individual patient, an individual batch prescription that includes a plurality of drug prescriptions for the individual patient, and a facility batch prescription that includes one or more drug prescriptions for each of a plurality of individual patients.

In response to receiving the prescription data for the first prescription, list order module 222 may determine, based on the promise time for the first prescription, a first position on a list of prescriptions for the first prescription. Rules data store 226 may store the list of prescriptions, as well as the model for handling and storing the various prescriptions. The list of prescriptions includes one or more prescriptions to be validated by a pharmacist and is sorted by a respective promise time for each of the one or more prescriptions to be validated. Each prescription in the one or more prescriptions to be validated was received by communication module 220 and from one of the plurality of client devices.

List order module 222 may place an indication of the first prescription in a first position on a list of prescriptions to create an updated list. Communication module 220 may, in response to creating the updated list, send the updated list to each computing device of the plurality of client devices.

Communication module 220 may then receive, from the second client device, a validation indication for the first prescription. The validation indication indicates that the prescription information has been reviewed and approved by a pharmacist. In response to receiving the validation indication for the first prescription, list order module 222 may remove the indication of the first prescription from the prescription list to create a second updated list. Communication module 220 may then send the second updated list to each client device of the plurality of client devices, and send a notification indicating the validity of the first prescription to at least the first client device of the plurality of client devices, potentially including each client device of the plurality of client devices located at the first geographic location.

In some instances, communication module 220 may receive, from the second client device, a hold indication for the first prescription. The hold indication indicates that there are one or more potential errors in the prescription information that has been reviewed by a pharmacist. In response to receiving the hold indication for the first prescription, list order module 222 may remove the indication of the first prescription from the prescription list to create a second updated list. Communication module 220 may send the second updated list to each client device of the plurality of client devices and send a notification indicating the one or more potential errors in the prescription information to at least the first client device of the plurality of client devices. These potential errors may include one or more of an instruction of a doctor not matching an entry in the prescription information, a missing entry in the prescription information, an entry in the prescription information that is classified as unclear, or an entry in the prescription information that does not follow medical guidelines for a drug prescribed in the prescription information.

In some instances, the updated list is sorted first by a priority for each prescription in the updated list and second by the promise time for each prescription in the updated list. The priority information could be based on an indication that a patient retrieving the prescription has arrived at the first geographic location, an indication of a shift change at the first geographic location, or an indication of a nursing home delivery occurring at a first time. Communication module 220 may receive, from the first client device, a priority adjustment indication for the first prescription, where the priority adjustment indication changes a priority for the first prescription from a low priority status to a high priority status (e.g., the high priority status indicating that the patient retrieving the prescription has arrived at the pharmacy, that a shift change is coming up at a pharmacy, or that a nursing home delivery is about to arrive). In response to receiving the priority adjustment indication for the first prescription, list order module 222 may determine, based on the promise time for the first prescription and the high priority status for the first prescription, a second position in the updated list for the first prescription and move the indication of the first prescription to the second position in the updated list to create a second updated list. Communication module 220 may send the second updated list to each client device of the plurality of client devices.

Communication module 220 may receive, from the second client device, a validation initiation indication for the first prescription, where the validation initiation indication indicates that a pharmacist at the second geographic location is reviewing (e.g., actively reviewing) the prescription information. In response to receiving the validation initiation indication for the first prescription, list order module 222 may remove the indication of the first prescription from the prescription list to create a second updated list. Communication module 220 may then send the second updated list to each client device of the plurality of client devices and send, to at least the first client device of the plurality of client devices, a notification indicating an identifier for the pharmacist at the second geographic location who is reviewing the first prescription.

Communication module 220 may include a log-in procedure for pharmacists to access the list of prescriptions. For instance, prior to receiving the prescription data for the first prescription, communication module 220 may receive, for each of the plurality of client devices, log-in information for a pharmacist at a same geographic location as the respective client device, wherein the log-in information comprises one or more of a username and password combination or biometric information.

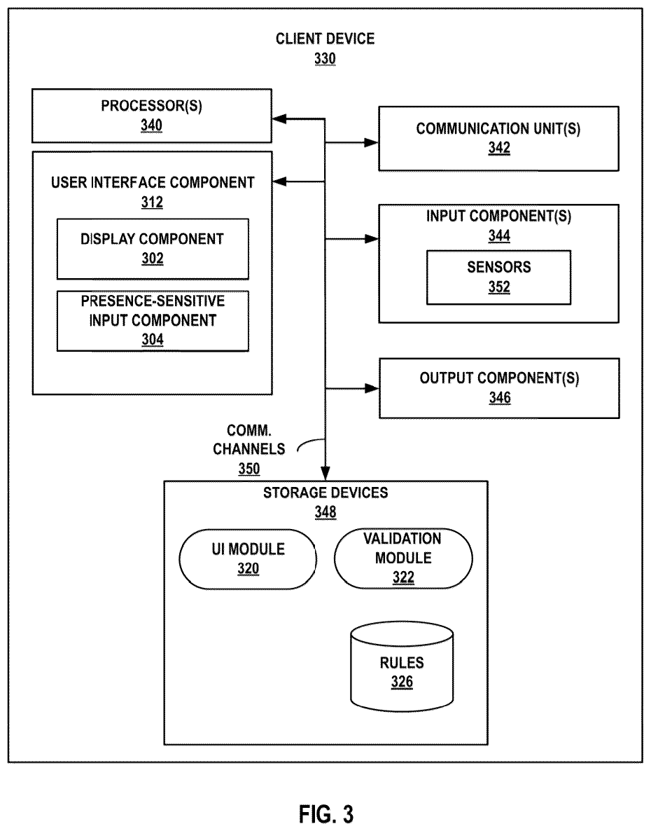

FIG. 3 is a block diagram illustrating a more detailed example of a client device configured to perform the techniques described herein. Client device 330 of FIG. 3 is described below as an example of any one of client devices 130A-130N of FIG. 1. FIG. 3 illustrates only one particular example of client device 330, and many other examples of client device 330 may be used in other instances and may include a subset of the components included in example client device 330 or may include additional components not shown in FIG. 3.

As shown in the example of FIG. 3, client device 330 includes user interface component (UIC) 312, one or more processors 340, one or more communication units 342, one or more input components 344, one or more output components 346, and one or more storage components 348. UIC 312 includes display component 302 and presence-sensitive input component 304. Storage components 348 of client device 330 include UI module 320, validation module 322, and rules data store 326.

One or more processors 340 may implement functionality and/or execute instructions associated with client device 330 to receive a list of prescriptions from a server device and to validate the information in those prescriptions, even if the prescription originated or is to be picked up in a different location. That is, processors 340 may implement functionality and/or execute instructions associated with client device 330 to remotely validate prescription information for pharmacies in a different location than client device 330.

Examples of processors 340 include application processors, display controllers, auxiliary processors, one or more sensor hubs, and any other hardware configure to function as a processor, a processing unit, or a processing device. Modules 320 and 322 may be operable by processors 340 to perform various actions, operations, or functions of client device 330. For example, processors 340 of client device 330 may retrieve and 19 execute instructions stored by storage components 348 that cause processors 340 to perform the operations described with respect to modules 320 and 322.

One or more storage components 348 within client device 330 may store information for processing during operation of client device 330 (e.g., client device 330 may store data accessed by modules 320 and 322 and rules data store 326 during execution at client device 330). In some examples, storage component 348 is a temporary memory, meaning that a primary purpose of storage component 348 is not long-term storage. Storage components 348 on client device 330 may be configured for short-term storage of information as volatile memory and therefore not retain stored contents if powered off. Examples of volatile memories include random access memories (RAM), dynamic random-access memories (DRAM), static random access memories (SRAM), and other forms of volatile memories known in the art.

Storage components 348, in some examples, also include one or more computer-readable storage media. Storage components 348 in some examples include one or more non-transitory computer-readable storage mediums. Storage components 348 may be configured to store larger amounts of information than typically stored by volatile memory. Storage components 348 may further be configured for long-term storage of information as non-volatile memory space and retain information after power on/off cycles. Examples of non-volatile memories include magnetic hard discs, optical discs, floppy discs, flash memories, or forms of electrically programmable memories (EPROM) or electrically erasable and programmable (EEPROM) memories. Storage components 348 may store program instructions and/or information (e.g., data) associated with modules 320 and 322 and rules data store 326. Storage components 348 may include a memory configured to store data or other information associated with modules 320 and 322 and rules data store 326.

Communication channels 350 may interconnect each of the components 312, 340, 342, 344, 346, and 348 for inter-component communications (physically, communicatively, and/or operatively). In some examples, communication channels 350 may include a system bus, a network connection, an inter-process communication data structure, or any other method for communicating data.

One or more communication units 342 of client device 330 may communicate with external devices via one or more wired and/or wireless networks by transmitting and/or receiving network signals on one or more networks. Examples of communication units 342 include a network interface card (e.g. such as an Ethernet card), an optical transceiver, a radio frequency transceiver, a GPS receiver, or any other type of device that can send and/or receive information. Other examples of communication units 342 may include short wave radios, cellular data radios, wireless network radios, as well as universal serial bus (USB) controllers.

One or more input components 344 of client device 330 may receive input. Examples of input are tactile, audio, and video input. Input components 344 of client device 330, in one example, includes a presence-sensitive input device (e.g., a touch sensitive screen, a PSD), mouse, keyboard, voice responsive system, camera, microphone or any other type of device for detecting input from a human or machine. In some examples, input components 344 may include one or more sensor components 352 one or more location sensors (GPS components, Wi-Fi components, cellular components), one or more temperature sensors, one or more movement sensors (e.g., accelerometers, gyros), one or more pressure sensors (e.g., barometer), one or more ambient light sensors, and one or more other sensors (e.g., infrared proximity sensor, hygrometer sensor, and the like). Other sensors, to name a few other non-limiting examples, may include a heart rate sensor, magnetometer, glucose sensor, olfactory sensor, compass sensor, step counter sensor.

One or more output components 346 of client device 330 may generate output in a selected modality. Examples of modalities may include a tactile notification, audible notification, visual notification, machine generated voice notification, or other modalities. Output components 346 of client device 330, in one example, includes a presence-sensitive display, sound card, video graphics adapter card, speaker, cathode ray tube (CRT) monitor, liquid crystal display (LCD), or any other type of device for generating output to a human or machine in a selected modality.

UIC 312 of client device 330 may include display component 302 and presence-sensitive input component 304. Display component 302 may be a screen at which information (e.g., a visual indication) is displayed by UIC 312 while presence-sensitive input component 304 may detect an object at and/or near display component 302.

While illustrated as an internal component of client device 330, UIC 312 may also represent an external component that shares a data path with client device 330 for transmitting and/or receiving input and output. For instance, in one example, UIC 312 represents a built-in component of client device 330 located within and physically connected to the external packaging of client device 330 (e.g., a screen on a mobile phone). In another example, UIC 312 represents an external component of client device 330 located outside and physically separated from the packaging or housing of client device 330 (e.g., a monitor, a projector, etc. that shares a wired and/or wireless data path with client device 330).

UIC 312 of client device 330 may detect two-dimensional and/or three-dimensional gestures as input from a user of client device 330. For instance, a sensor of UIC 312 may detect a user's movement (e.g., moving a hand, an arm, a pen, a stylus, a tactile object, etc.) within a threshold distance of the sensor of UIC 312. UIC 312 may determine a two or three-dimensional vector representation of the movement and correlate the vector representation to a gesture input (e.g., a hand-wave, a pinch, a clap, a pen stroke, etc.) that has multiple dimensions. In other words, UIC 312 can detect a multi-dimension gesture without requiring the user to gesture at or near a screen or surface at which UIC 312 outputs information for display. Instead, UIC 312 can detect a multi-dimensional gesture performed at or near a sensor which may or may not be located near the screen or surface at which UIC 312 outputs information for display.

In accordance with the techniques described herein, UI module 320 may receive, from a server device, a list of prescriptions comprising one or more prescriptions to be validated by a pharmacist. The list of prescriptions is sorted by at least a respective promise time for each of the one or more prescriptions to be validated, where each prescription in the one or more prescriptions to be validated was received by the server device from one of a plurality of client devices that includes client device 330. Client device 330 is located in a first geographic location. A first prescription in the list of prescriptions was sent to the server device by a second client device of the plurality of client devices, and with the second computing device being located in a second geographic location different than the first geographic location.

The prescription information may include one or more of a patient name, a prescriber name, a prescription type, a fill type, a patient date of birth, a drug name, a drug strength, a drug form, a quantity written, a day supply, a refill amount, a drug application instruction, a prescriber license, and a prescription date. The first prescription may be any one or more of an individual prescription for an individual patient, an individual batch prescription that includes a plurality of drug prescriptions for the individual patient, and a facility batch prescription that includes one or more drug prescriptions for each of a plurality of individual patients.

UI module 320 may output, for display on UIC 312 or output components 346, at least a portion of the list of prescriptions in a first graphical user interface. In the first graphical user interface, the list of prescriptions may be sorted first by a priority for each prescription in the list of prescriptions and second by the promise time for each prescription in the list of prescriptions. The priority may be one or more of an indication that a patient retrieving the prescription has arrived at the first geographic location, an indication of a shift change at the first geographic location, an indication of a nursing home delivery occurring at a first time, or a low priority status.

UI module 320 may receive an indication of user input selecting the first prescription from the list of prescriptions in the graphical user interface. In response to receiving the indication of user input selecting the first prescription, UI module 320 may output, for display on UIC 312 or output components 346, prescription information to be validated for the first prescription in a second graphical user interface.

In some instances, UI module 320 may further receive an indication of a second user input that comprises a determination for the first prescription. The determination may be one of a validation of the prescription information for the first prescription or an indication of one or more potential errors in the prescription information for the first prescription. In response to receiving the indication of the second user input, UI module 320 may send, to the server device, the determination for the first prescription. The one or potential errors may include one or more of an instruction of a doctor not matching an entry in the prescription information, a missing entry in the prescription information, an entry in the prescription information that is classified as unclear, or an entry in the prescription information that does not follow medical guidelines for a drug prescribed in the prescription information.

In response to sending the determination for the first prescription to the server device, UI module 320 may then output, for display on UIC 312 or output components 346, prescription information to be validated for a second prescription in a third graphical user interface. The second prescription may be a prescription in the prescription list with a highest position in the prescription list, enabling client device to continue cycling through the prescriptions in the list of prescriptions from all of the pharmacies based on which prescriptions are expected to be picked up next, thereby optimizing the workflow.

In response to sending the determination for the first prescription to the server device, UI module 320 may receive an updated list from the server device. The updated list may include a set of one or more prescriptions to be validated by a pharmacist, but the updated list may not include the first prescription. UI module 320 may output, for display on UIC 312 or output components 346, at least a portion of the updated list in a third graphical user interface.

In some instances, a first portion of the second graphical user interface includes an electronic prescription received from a medical provider. A second portion of the second graphical user interface includes a plurality of data fields. Validation module 322 may extract information from the electronic prescription and populate one or more of the data fields in the plurality of data fields with the information extracted from the electronic prescription. UI module 320 may detect an indication of a second user input at a first field of the plurality of data fields in the second portion of the second graphical user interface. The second user input may be a mouse click of the first field, a tap from an input tool on the first field, or a mouse hover over the first field. In response to detecting the indication of the second user input at the first field, UI module 320 may highlight, in the second graphical user interface, both the first field in the second portion of the second graphical user interface and an area of the electronic prescription that includes the information used to populate the first field.

While causing UIC 312 or output components 346 to output the first graphical user interface, UI module 320 may receive an indication of a second user input selecting a data field within the first graphical user interface. UI module 320 may sort the list of prescriptions based on the selected data field to create a resorted list of prescriptions. UI module 320 may then output, for display on UIC 312 or output components 346, at least a portion of the resorted list of prescriptions in a third graphical user interface.

UI module 320 may receive, from the server device, a hold indication for a second prescription. The second prescription is for a patient at the first geographic location, and the hold indication indicates that there are one or more potential errors in the prescription information for the second that has been reviewed by a pharmacist in the second geographic location. In response to receiving the hold indication for the first prescription, UI module 320 may output, for display on UIC 312 or output components 346, the one or more potential errors in the second prescription.

To log in to client device 330, UI module 320 may receive log-in information for a pharmacist at the first geographic location. The log-in information may include one or more of a username and password combination or biometric information (e.g., fingerprint scan, face scan, ocular scan, etc.). UI module 320 may send the log-in information to the server device for verification.

In accordance with some techniques described herein, validation module 322 may create a pharmaceutical profile for a patient in rules data store 326. The pharmaceutical profile may include information regarding one or more of one or more active prescriptions for the patient, one or more inactive past prescriptions for the patient, one or more diagnosis codes for the patient, prescription directions for the one or more active prescriptions for the patient, allergy information for the patient, clinical interventions for the patient, insurance information for the patient, a preferred store for the patient, or demographic information for the patient. UI module 320 may receive real-time prescription information for the patient. Validation module 322 may generate an intervention plan for the patient based on the pharmaceutical profile for the patient and the real-time prescription information for the patient.

For instance, in generating the intervention plan, validation module 322 may compare the real-time prescription information with one or more aspects of the pharmaceutical profile. Validation module 322 may then determine whether the real-time prescription information is compatible with the pharmaceutical profile to ensure that the prescription is proper for that particular patient. Responsive to determining that the real-time prescription information is compatible with the pharmaceutical profile, validation module 322 may generate the intervention plan.

In some instances, the intervention plan may include one or more actions to be taken by either a pharmacy or the patient when the patient arrives at a pharmacy to pick up a current prescription associated with the real-time prescription information. The one or more actions could include any one or more of providing instructions for consuming the current prescription, providing details regarding how the current prescription integrates with the one or more active prescriptions for the patient, providing details regarding additional side effects due to the one or more diagnosis codes for the patient, providing details regarding how the current prescription integrates with the one or more clinical interventions for the patient, determining a need for a co-pay based on the insurance information for the patient, providing recommendations based on the one or more inactive prescriptions, and providing details regarding additional side effects due to the demographic information of the patient.

Client device 330 may update the pharmaceutical profile. For instance, UI module 320 may receive an indication of user input altering one or more aspects of the pharmaceutical profile.

UI module 320 may generate warnings. For instance, responsive to validation module 322 determining that the real-time prescription information is not compatible with the pharmaceutical profile, UI module 320 may generate and output a warning indicating how the real-time prescription information is not compatible with the pharmaceutical profile.

UI module 320 may further receive a list of one or more past prescriptions for the patient. For each of the one or more past prescriptions in the list, validation module 322 may determine whether the respective past prescription has been renewed in the past 30 or 90 days. Responsive to determining that the respective past prescription has been renewed in the past 30 or 90 days, validation module 322 may classify the respective prescription as an active prescription in the pharmaceutical profile for the patient. Conversely, responsive to determining that the respective past prescription has not been renewed in the past 30 or 90 days, validation module 322 may classify the respective prescription as an inactive prescription in the pharmaceutical profile for the patient.

Validation module 322 may also generate clinical information regarding the real-time prescription information. UI module 320 may output the clinical information directly within a user interface for a dispensing application.

Validation module 322 may further compare the pharmaceutical profile to respective criteria for each of one or more clinical intervention trials on each of one or more clinical platforms. Validation module 322 may determine one or more qualified clinical intervention trials as the one or more clinical intervention trials in the one or more clinical platforms that the patient qualifies for based on the pharmaceutical profile for the patient. UI module 320 may then present the one or more qualified clinical intervention trials and information regarding the one or more qualified clinical intervention trials within a user interface for a dispensing application.

In some instances, UI module 320 may receive patient information from one or more of an insurance payor or an employer group. Validation module 322 may then generate the pharmaceutical profile for the patient based on the patient information.

In some instances, the pharmaceutical profile may further include additional information used to generate the intervention plan. Examples of such additional information could include any one or more of a drug history, a treatment direction history, a drug quantity history, a family history, a laboratory assessment history, a vital assessment history, a social behavior, an allergy history, immunization history, patient height, patient weight, and previous recommendations. In this way, the techniques described herein may include the process of verifying, collecting, and clarifying in an electronic format an accurate list of all medications a patient is taking. Examples include drug use, directions, quantity, family history, lab and/or vital assessments, social behaviors, (e.g., tobacco use, alcohol use, caffeine consumption, and exercise), allergies, and other prior recommendations. Including this information in the pharmaceutical profile may assist the system in avoiding medical errors and hospital readmissions while improving patient outcomes. Additionally, these techniques assist with transitional care management. Some initiatives, such as Medicare initiatives, are designed to improve healthcare delivery as well as lower costs so as to keep patients healthier and prevent relapses and readmissions. Through connectivity and the API with outside systems, computing device 310 may be notified of patient discharges from hospitals. Any discrete data elements known to the pharmacy will be pulled into the assessment to provide a robust review yet staying efficient in time spent reducing total cost of care. Examples of discrete data elements are medications, immunizations, height, weight, and blood pressure, among other things.

In some instances, validation module 322 may further schedule, based on the intervention plan, a follow-up appointment for the patient. In this way, computing device 310 may provide a centralized web-based appointment scheduling tool. This includes the ability to document all phone or in-person services and conversations. Computing device 310 allows user administrators to set up and publish daily/weekly/monthly, etc., schedules for medical recommendation assessments. Real-time reporting is also used to assist monitoring the progress of programs and sending appointment reminders and missed appointment calls to the patient. Examples include date, time slot, status, patient information, schedule type, and job area, among other things. In some instances, UI module 320 may further issue, for display on UIC 312, one or more prompts during the follow-up appointment. Validation module 322 may update the pharmaceutical profile based on user-entered replies to the one or more prompts to create an updated pharmaceutical profile and generate an updated intervention plan based on the updated pharmaceutical profile.

In some examples, in generating the intervention plan, validation module 322 may determine, based on the pharmaceutical profile for the patient, whether the patient qualifies for particular preventative treatment program. The eligibility for the particular preventative treatment program may be defined by the patient meeting particular criteria. Responsive to determining that the patient qualifies for the particular preventative treatment program, validation module 322 may include one or more elements of the particular preventative treatment program into the intervention plan. This may be used, for instance, as a user and patient guided assessment to work with Medicare patients developing or updating a personalized prevention plan. This plan may help prevent illness based on current health and risk factors. Patients meeting qualifying criteria may have an eligibility check completed prior to review to ensure eligibility of services for payment purposes. Through the techniques described herein, there will be pre-populated recommendations presented for the patient. Any discrete data elements known to the pharmacy through our dispensing system or connectivity with other vendors will be pulled into the assessment to provide a robust efficient review in time spent reducing total cost of care. Examples of discrete data elements are medications, immunizations, height, weight, and blood pressure, among other things. A medical physician may provide a final sign off and may review all visits. Billing may occur within the platform or computing device 310 may generate a billing file can be exported to the appropriate entity.

In some instances, UI module 320 may further output the intervention plan for review by a medical physician. For example, there may be a queue imbedded within the platform for a medical physician to review completed services. As such, computing device 310 provides the ability for a required physician final review where the pharmacist is the care manager. Examples of such services are annual wellness visits, advance care planning, transitional care management, remote patient monitoring, and chronic care management, among other things.

In some instances, the pharmaceutical profile may include at least a list of each medication being taken by the patient. In such instances, the intervention plan may further include an assessment of each medication being taken by the patient and a determination of whether each medication being taken by the patient is appropriate for the patient based on one or more additional characteristics of the patient in the pharmaceutical profile. In this way, computing device 310 maintains a standard of care that ensures each patient's medications (whether they are prescription, nonprescription, alternative, traditional, vitamins, or nutritional supplements) are individually assessed to determine that each medication is appropriate for the patient, effective for the medical condition, safe given the comorbidities and other medications being taken, and able to be taken by the patient as intended. Comprehensive medication management (CMM) includes an individualized care plan that achieves the intended goals of therapy with appropriate follow-up to determine actual patient outcomes. This all occurs because the patient understands, agrees with, and actively participates in the treatment regimen, thus optimizing each patient's medication experience and clinical outcomes. A pharmacist practicing CMM may be, at times, practicing under a Collaborative Practice Agreement (CPA), which allows the pharmacist to initiate and modify pharmacotherapy under the authority of a prescriber.

CMM builds on CMR by having a broader focus that includes other elements of the patient's care, like clinical outcomes and personal goals of therapy. CMM both incorporates patient history into recommendations and makes recommendations intended to impact elements of the patient's history through measurable clinical outcomes. A readily identifiable example of CMM is anticoagulation management. In pharmacist-led anticoagulation clinics, the pharmacist acts as “provider” with face-to-face appointments where the pharmacist gathers a problem-based patient history, performs point-of-care (POC) testing, and recommends dose adjustments directly to the patient. Pharmacist-led diabetes clinics are another example.

In some instances, the pharmaceutical profile may further include information gathered by a pharmacist during an assessment of the patient during a patient encounter. In such examples, the intervention plan may further include one or more of a pharmacological treatment recommendation and a non-pharmacological referral. For example, in a typical assessment, pharmacists may use a “4R” Assessment, including review, risk, resources, and referral, with the process repeating as needed upon revisits. The assessment tool provided herein takes predictive information or information present at the time of a patient encounter and evaluates the risk, determines resources and referrals. A pharmacy may partner with local community resources to provide referrals or care. The pharmacy may have a specialized healthcare member coming into the pharmacy once a month for testing (e.g., hearing or eye evaluations) of the customers at the pharmacy. Patient can make appointments for these services with the pharmacist.

The benefits for the provider of the service is to assist identification of risks/problems, understand how to be a resource, and refer to any specialize care or products. For the patient these techniques help them understand and easily comprehend the visit, including their review, risk, resources, and referrals. The assessment covers both pharmacological and non-pharmacological recommendations and referrals. Overall, this capability streamlines the process for the provider to find appropriate resources and referrals.

This assessment tool allows the pharmacist to continue to be the most accessible health care personnel providing the best in class patient care. This tool also allows the pharmacist to be part of the patient's care team working with other healthcare professionals reducing total cost of care and improving health outcomes. A use case example of this referral/recommendation tool is when the patient thinks they might be coming down with something. The pharmacist brings the patient into the consultation room for evaluation. The pharmacist goes through a series of assessment questions to help understand why the patient is not feeling well. The pharmacist goes over these results with the patient and refers them to their primary care for further evaluation or the pharmacist prescribes a starter dose of applicable medication due to their provider status or through a collaborative practice agreement.

In some examples, in generating the intervention plan, validation module 322 may identify, based at least in part on the pharmaceutical profile for the patient, one or more drug therapy problems for one or more of the patient or the pharmacist. Validation module 322 may then include the one or more drug therapy problems in the intervention plan. This technique may look at all the clinical data that is available for patient and helps prioritizes their Drug Therapy Problems (DTP) for both the provider and patient. This clinical data would include data the pharmacy may have from pharmacogenomics (PGx) testing. This may streamline a pharmacist's efforts to determine which DTPs to work on with a patient and how many to work on. The pharmacist may address DTPs in an order of importance and not try to change too many things at once. For instance, the patient may complete a Comprehensive Medication Review (CMR). The CMR may indicate that the patient presents for 8 DTPs. The goal would be to work in order of importance so side effects can be monitored and changes are effective.

In some instances, UI module 320 may additionally prompt the pharmacist for performance of one or more steps of a stages-of-change assessment for a behavior in the pharmaceutical profile for the patient. UI module 320 may receive one or more indications of user input indicating results of the one or more steps of the stages-of-change assessment for the behavior. Based at least in part on the results of the one or more steps of the stages-of-change assessment and the behavior, validation module 322 may generate, and UI module 320 may provide and output, one or more of pharmaceutical content and clinical services content for the pharmacist to utilize in treating the patient for a change in the behavior.

A concept behind the techniques described herein is the real-time journey tracking and digital nudging that can be done to afford patient accountability and hence predictability, notwithstanding intended behaviors around drug use, especially in chronic or orphan drug applications. The techniques described herein may capture and store in a database, Prochaska stages-of-change assessments collected by a plurality of digital methods and times. The TTM has found that individuals move through a series of stages-precontemplation (PC), contemplation (C), preparation (PR), action (A), and maintenance (M)-in the adoption of healthy behaviors or cessation of unhealthy ones. Ideally the assessment would be done in a single setting, but pieces of the assessment could be done over time and aggregated into a unified profile. Computing device 310 may then use partial or completed assessments to create and deliver psychographically aligned pharmaceutical or clinical services content (education, marketing, etc.) to drive idea adoption and/or more accurately drive better adherence, compliance, selfcare services and subsequently track a patient's journey.

For pharmacists the insights around “readiness to change” (RTC) will help to lower costs and time in trying to persuade a patient who isn't intrinsically ready to “own” the changed behavior. Correspondingly, pharmaceutical or payer customers engaging pharmacists in clinical services programs with RTC insights, will have more convincing assurance of patient outcomes and/or the understanding of what it takes to nudge patients to be more accountable hence lowering overall long-term spending. Pharmacists using RTC insights could, for the first time ever, also offer cost-reduction guarantees (outcomes) to payers or pharmaceutical customers subsidizing programs. Lastly, consumers considering adopting tailored clinical, nutritional, remote monitoring or tele-services, will finally be more inclined to do so because the content messaging based on RTC factors, will be more personalized (versus generalized). This personalization will lower the adoption friction and increase the probability of adherence and compliance of such clinical services.

Patient journey endpoints (clinical outcomes and/or out-reach or treatment costs), based on RTC factors, could be configurable (retrospectively or prospectively) to autonomously adjust the number of inputs (dosing, outreach, incentives, etc.) to match the patient's readiness to change. This same journey data could be used as a leading indicator to profile (score, handicap, etc.) eligibility for payer or pharmaceutical programs as part of a financial assurance for same companies seeking guaranteed performance.

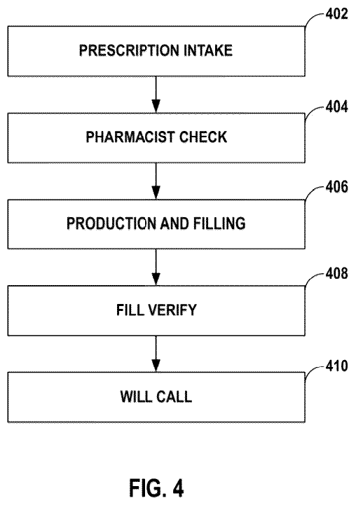

FIG. 4 is a flow diagram illustrating example techniques performed by one or more devices within the system described herein. The techniques of FIG. 4 may be performed by one or more processors of a computing device, such as server device 110 of FIG. 1, one of client devices 130 of FIG. 1, server device 210 illustrated in FIG. 2, or client device 330 of FIG. 3. For purposes of illustration only, the techniques of FIGS. 4A and 4B are described within the context of server device 210 of FIG. 2 and client device 330 of FIG. 3, although computing devices having configurations different than that of server device 210 and client device 330 may perform the techniques of FIG. 4.

In accordance with the techniques described herein, client device 330 may receive a prescription (402) such as from a medical professional. The prescription includes medication information and patient information. Client device 330 sends that prescription information to server device 210, which adds the prescription to a list of prescriptions stored at server device 210 and accessible by client device 330 and a number of other client devices at other locations. Client device 330 may initiate a pharmacist check process (404), which retrieves a first prescription from the list of prescriptions at server device 210. The first prescription may not be a prescription for pickup at a location of client device 330, but instead the first prescription may be for medication at a different location. Once client device 330 completes the check, a pharmacist at the location where the medication is to be picked up may complete production and filling of the prescription (406), may complete the fill verification for the prescription (408), and may place the prescription in the will call queue for pickup (410).

The techniques described herein include a new workflow process to process prescriptions and to increase efficiencies in the pharmacy. The ability to workload balance is predicated on splitting the pharmacist checking process into two steps: Pharmacist Check and Fill Verify. Pharmacist Check (or PC) is the step where the pharmacist checks that the data entered by the technicians matches what was on the prescription hardcopy, as well as reviews any DUR items such as patient allergies or drug interactions. This step can be done by a pharmacist at a remote store. Once that step is completed, the prescription may go to the dispensing application at the originating store to be filled by the technicians at that store. Once the technicians are finished filling the prescription, the script may be available in Fill Verify (FV) for the pharmacist at the store to check. At this stage, they are checking that the prescription has been filled correctly; with the right drug, quantity, etc.

The Workflow platform may be divided into eight sections:

A first section may be PC Verify, which can be used in conjunction with PC Queue. Prescriptions to verify feed to this queue from the PC Queue based on expected fill time. On approval of the current Rx, the next Rx in queue may display. This option may be helpful with Workload Balancing for the off-site pharmacist who is verifying data entry from one or multiple pharmacies.

A second section may be PC Queue. All prescriptions waiting for Pharmacist verification of data entry display in the PC Queue, prioritized by time of pick up.

A third section may be Fill Verify. All prescriptions waiting for Pharmacist verification of filled Rx's display in this queue.

A fourth section may be Admits Queue. Long Term Care Admit Records waiting for pharmacist verification display here. Admit orders can be verified from this queue or from the PC Queue.

A fifth section may be Search. This function allows the user to search prescriptions for a specific patient, facility, prescription number, or date range.

A sixth section may be Specialty, or a specific queue for specialty prescriptions

A seventh section may be Verification. Certain states' boards of pharmacy may require a second quality assurance check on prescriptions where the pharmacist may compare the original, or image of the original written prescription to the information entered into the computer and documenting the completion and accuracy of this comparison.

This process may not occur prior to two hours after the prescription has been initially certified, unless it is completed by a second individual pharmacist as soon as possible after the initial certification has occurred. The process may be completed within 72 hours.

An eighth section may be Clinical. This queue houses all of the clinical interventions generated through the artificial intelligence model. Within each patient, clinical conversations are presented that the pharmacists can discuss with the patient.

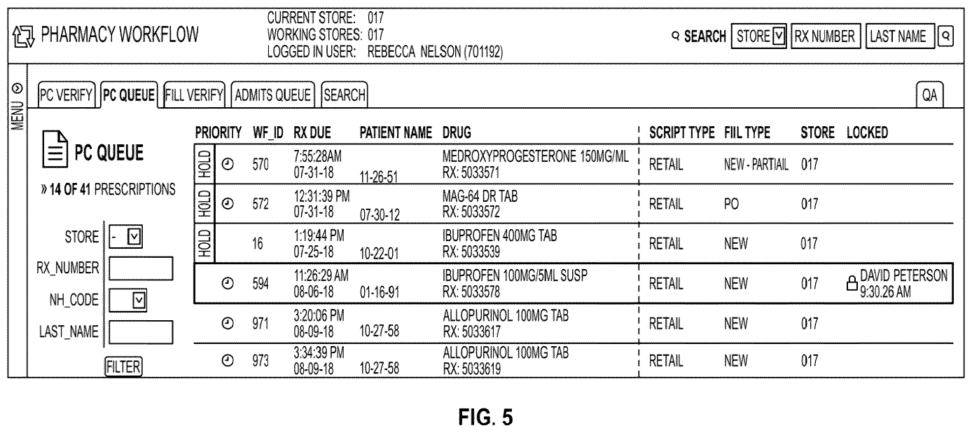

FIG. 5 is an example portion of a graphical user interface showing an example prescription fill list, in accordance with one or more of the techniques described herein. The PC Queue displays all prescriptions waiting for pharmacist verification of the data entry. They are prioritized by expected pick up/delivery time as entered in TWRx. That time displays in the RX DUE column. The PRIORITY column may display a clock icon for patients that are waiting.

Prescriptions can be placed on a HOLD for any reason determined by the pharmacist. For example, if the pharmacist may be trying to contact the prescriber for more information or clarification of an order. If another pharmacist is working on a prescription, their name may display in the LOCKED column and no one else can access that prescription.

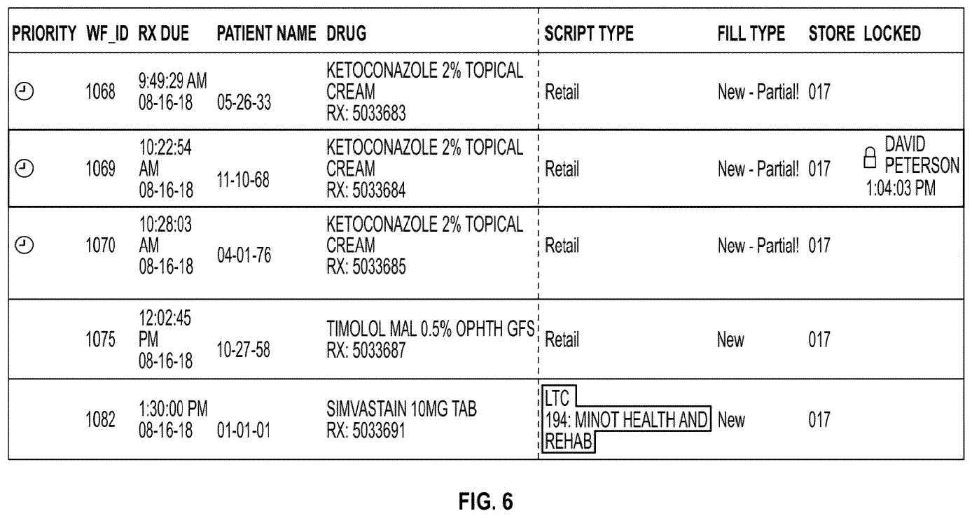

FIG. 6 is an example portion of a graphical user interface showing an example prescription fill list with a long term care option, in accordance with one or more of the techniques described herein. The SCRIPT TYPE column displays if the order is Retail or Long Term Care. If Long Term Care, the name of the facility may display. In the FILL TYPE column, one of the following types may display: New, New—Specialty, New—IOU, New—Partial, Refill, Refill—Specialty, Refill—IOU, Refill-Partial, Refill with Changes, Refill with Changes—IOU, Refill with Changes—Partial, PO (Profile Only), and Full Quantity Remotely Filled.

The system may include a reminder mechanism. Patients that are waiting display the Clock icon in the PRIORITY column, prescriptions placed on hold display with Yellow Highlight, and prescriptions being worked by another user are locked.

If the user is not using the PC Verify feeder functions to verify data entry on prescription orders, in the PC Queue tab, click on the specific Rx to verify. The PC VERIFY tab may auto-feed prescriptions to the pharmacist to verify based on the Rx Due time. The data entered from the dispensing system displays on the left hand side of the screen and the hard copy image from the doctor/physician displays on the right. The user may verify all data was entered to the written hard copy, as well as use the MAGNIFIER or ROTATE options in the upper right hand corner to enlarge or rotate the hard copy image as needed.

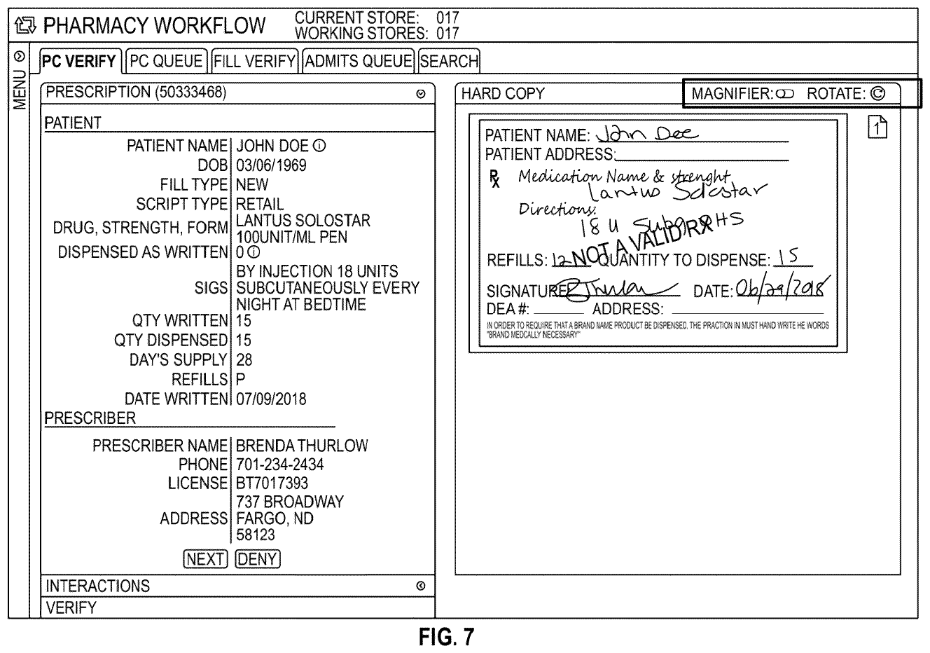

FIG. 7 is an example portion of a graphical user interface showing an example image prescription verification page, in accordance with one or more of the techniques described herein. On prescriptions that are transmitted via ERx, the system displays the information from the ERx in the Hard Copy window. As the user hovers the mouse over the data fields on the left, it may highlight the corresponding fields on the right. This was done to improve speed and accuracy of the ERx checking process.

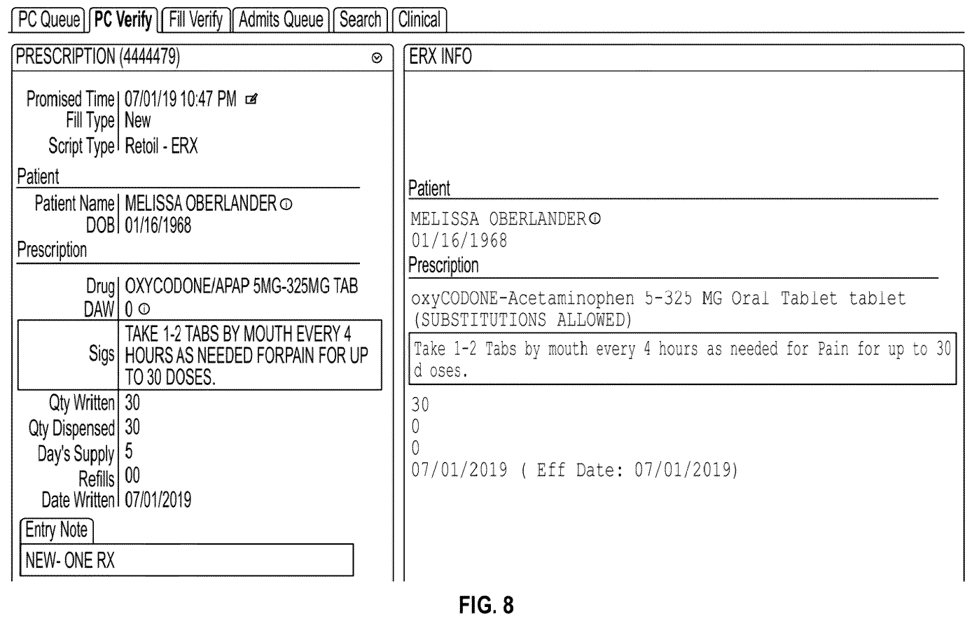

FIG. 8 is an example portion of a graphical user interface showing an example text prescription verification page, in accordance with one or more of the techniques described herein. The information icon next to the patient's name displays the patient's address. Click on the icon to access this information.

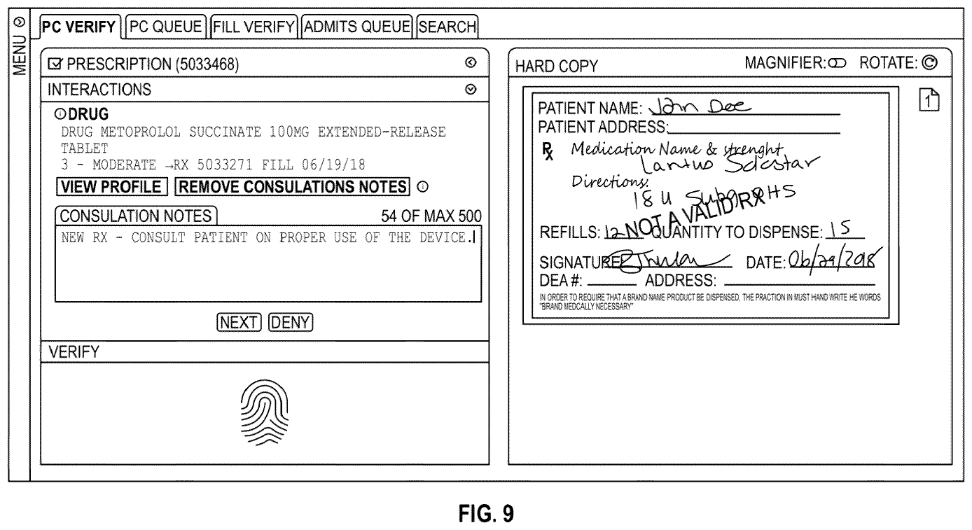

FIG. 9 is an example portion of a graphical user interface showing an example prescription image with a consultation notes field, in accordance with one or more of the techniques described herein. The information icon next to the Dispense As Written (DAW) field may display the DAW codes and meanings. The DAW can also be viewed from the Fill Verify Queue by clicking on SHOW PC VERIFY. For Long Term Care orders, the information icon may display next to the NH Code. Clicking on this icon may display certain parameters for the facility.

For approving data entry, if the data entry on the prescription order is correct, click on the NEXT button to view Interactions. In the Drug Utilization Review screen, interactions may display along with the severity level of each interaction. Click on the information icon next to each interaction, or duplicate therapy for more information.

If any consultation notes need to be attached to the prescription order, click on ADD CONSULTATION NOTES. Click on the information icon next to ADD CONSULTATION NOTES for further instruction on Notes. Enter the consultation notes then click NEXT. Consultation notes, if entered, may appear at Fill Verification where they can be printed and attached to the exterior of the patient's bag. A flag may be sent to POS that a consultation should be conducted, and that information can be found on the attached note.

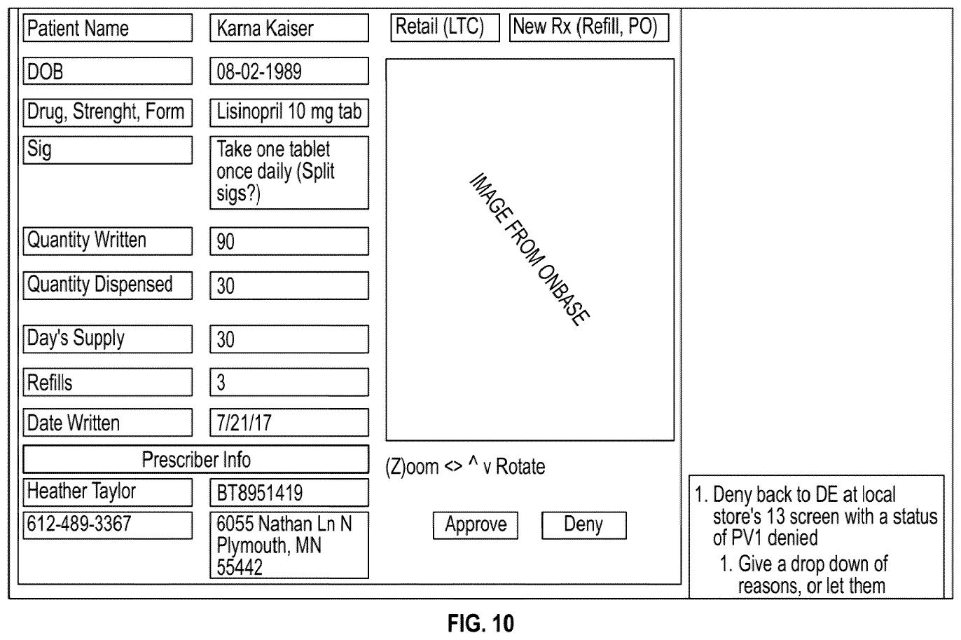

FIG. 10 is an example portion of a graphical user interface showing an example prescription entry page, in accordance with one or more of the techniques described herein.

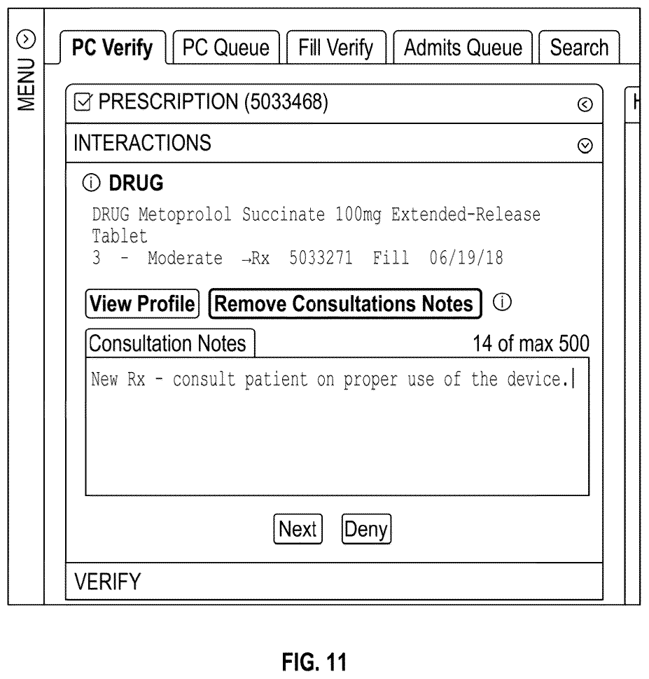

FIG. 11 is an example portion of a graphical user interface showing an example button to remove consultation notes, in accordance with one or more of the techniques described herein. Consultation notes can also be removed at this point if needed by clicking on REMOVE CONSULTATION NOTES. After the data entry has been confirmed, use the biometric scanner to certify the verification.

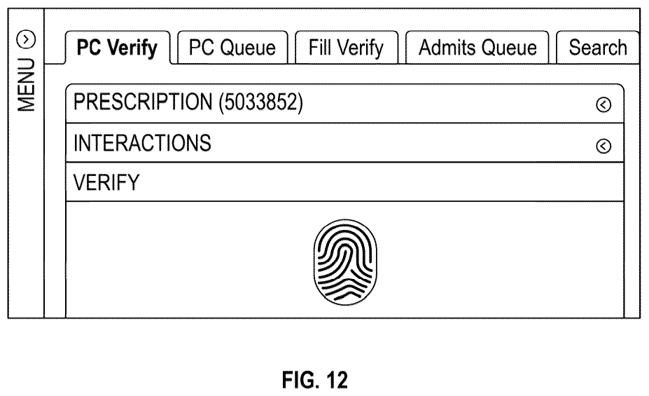

FIG. 12 is an example portion of a graphical user interface showing an example biometric verification request, in accordance with one or more of the techniques described herein.

Prescriptions that may require clarification or additional information from the prescriber, can be placed on CLINICAL HOLD. Click on the CLINICAL HOLD button. Enter any notes explaining the reason for the hold and click PUT ON CLINICAL HOLD.

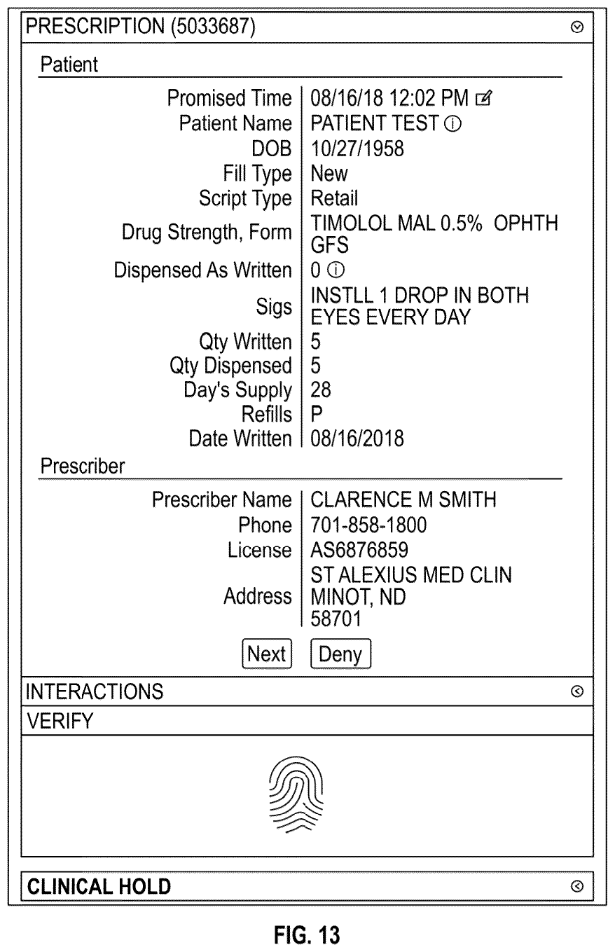

FIG. 13 is an example portion of a graphical user interface showing an example prescription with a clinical hold and a biometric verification request, in accordance with one or more of the techniques described herein.

The note can be viewed any time by clicking on the prescription from PC Queue. To release the prescription from HOLD, in the PC Queue, click on the prescription. Enter any additional notes and click SAVE. Then click RELEASE FROM CLINICAL HOLD. To change the priority time of expected pickup, open the prescription order in PC Queue and click on the pen icon next to the PROMISED TIME above the patient name. Select the new time and click SAVE.

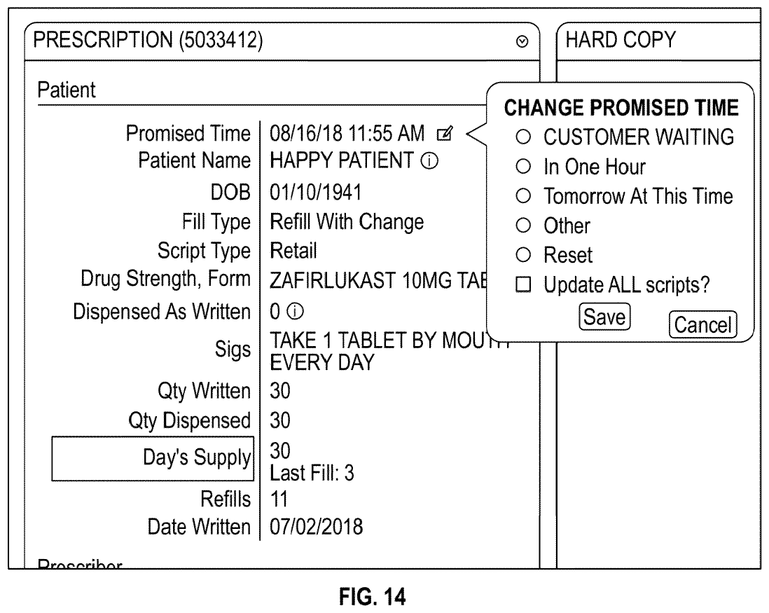

FIG. 14 is an example portion of a graphical user interface showing an example dialog box to change the promised prescription time, in accordance with one or more of the techniques described herein. When verifying a controlled substance prescription, the pharmacist may verify that they have examined the physical hard copy of the prescription unless it was electronically received.

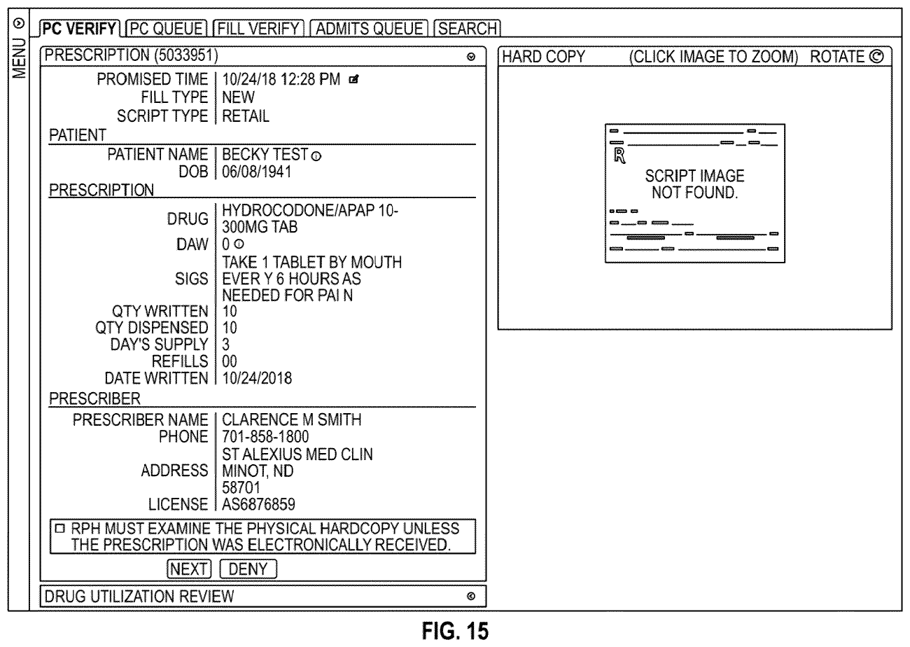

FIG. 15 is an example portion of a graphical user interface showing an example page to verify a prescription for a controlled substance, in accordance with one or more of the techniques described herein. When verifying an electronic prescription, the electronic data received displays as the prescription image.

For Fill Verify, when the Production Technician has filled the order in the dispensing system, it moves into the Fill Verify Queue. The same SCRIPT TYPE and FILL TYPE options may display based on the order type.

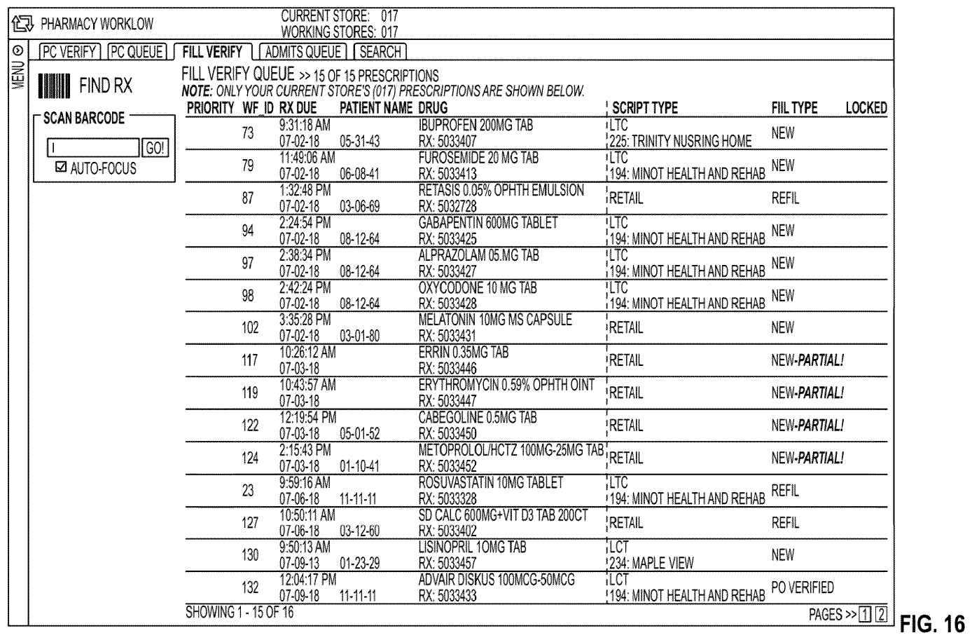

FIG. 16 is an example portion of a graphical user interface showing an example fill verify queue, in accordance with one or more of the techniques described herein. To verify a script, the user has the option to scan the barcode from the vial label or click on the patient's prescription to verify. The prescription may display along with the pill/product image.

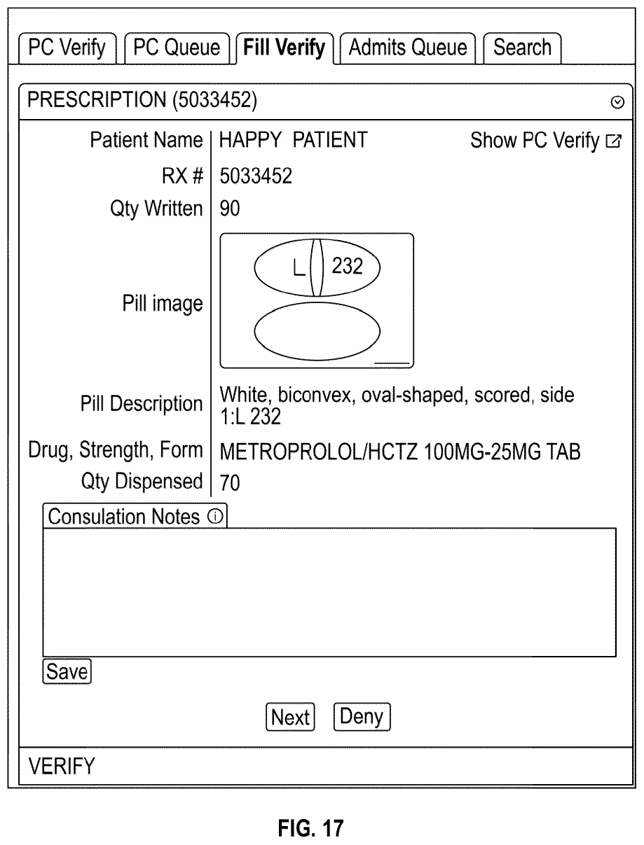

FIG. 17 is an example portion of a graphical user interface showing an example prescription pill image, in accordance with one or more of the techniques described herein. If the fill is a partial IOU, the quantity to dispense may be less than QTY Written and may be indicated as a partial in the FILL TYPE on the main screen.

When verifying a fill for an oral liquid product, the user may be prompted to verify that the stock bottle from which the liquid was withdrawn was checked. Consultation notes may display if entered at PC Check, and notes can be added at this point also. The pharmacist can continue with the approval of the fill or deny the fill.

To DENY the fill, click DENY. Then choose the reason for the denial and add notes. Click DENY to move the prescription back to the FILL QUEUE for the filler to re-do. The reason for the denial/rejection may display with the prescription in the Fill Queue. If the order needs to be denied back to data entry, click on SHOW PC VERIFY. The original PC Verify screen may display. Click on SEND ME TO PC VERIFY TO REJECT.

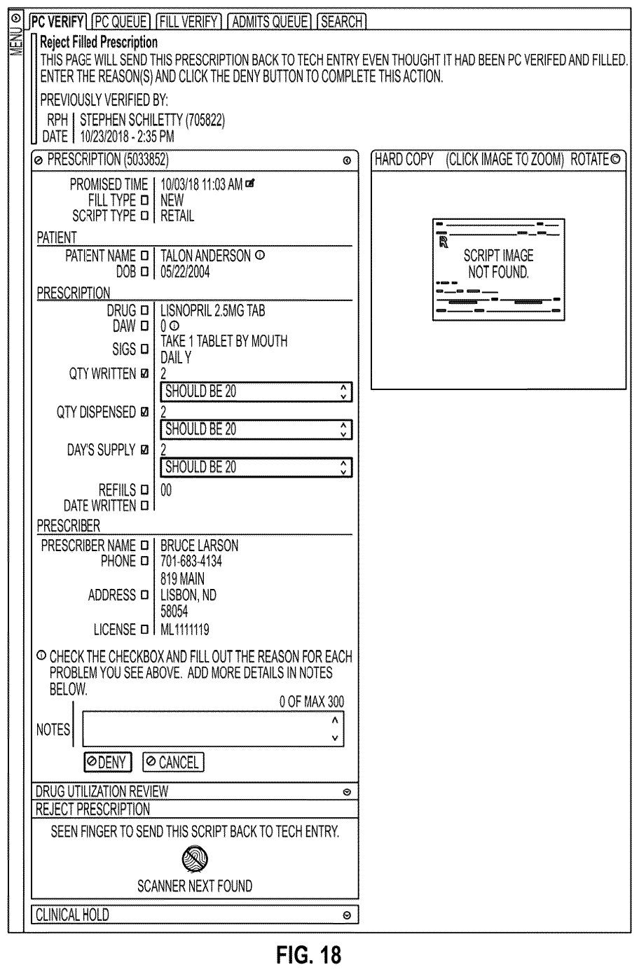

FIG. 18 is an example portion of a graphical user interface showing an example denial of a prescription, in accordance with one or more of the techniques described herein. To deny a prescription, click on DENY. Then check the boxes on the areas that may need to be corrected, enter the correct information, and then click DENY. This may send the prescription back to the dispensing system. To approve a fill, click on NEXT and use the biometrics to certify the fill.

If the order is Long Term Care and packaged in a blister card, an image of the FRONT view of the card may display in the Fill Verify Queue. The HOA and QTY information may display in the LTC CARDS field. If there are multiple cards filled for the prescription order, they may all display. The HOA for each card may display in the LTC CARDS fields as well as on the image of each card. The number of pills per blister may display in each blister accordingly. Half tabs may display as ‘0.50’

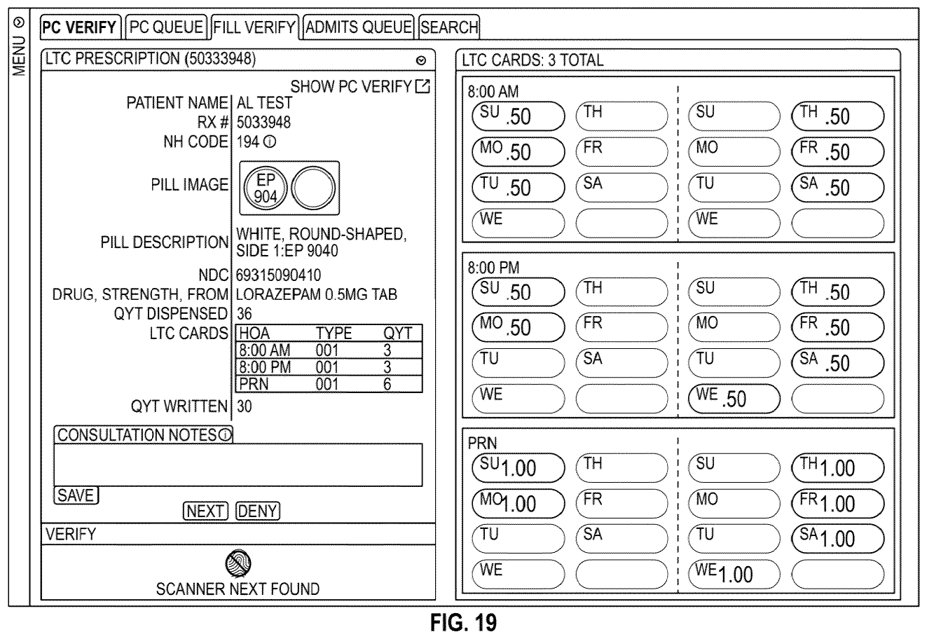

FIG. 19 is an example portion of a graphical user interface showing an example blister packet verification with both full and half tablets, in accordance with one or more of the techniques described herein. The appropriate card(s) may display depending on if it is 14-Day EasyPak or 30-Day Accuflex. The Admits Queue may display all new patients admitted today to a Long Term Care facility.

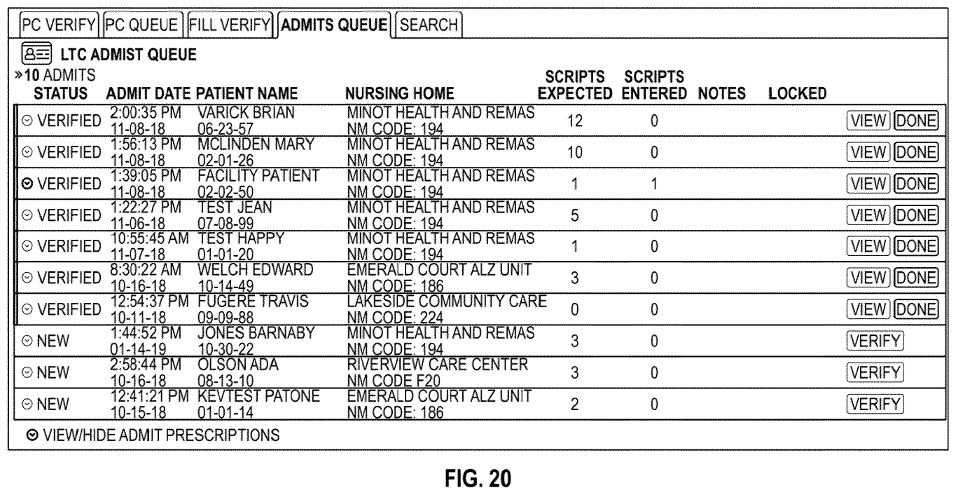

FIG. 20 is an example portion of a graphical user interface showing an example prescription fill list for long term care admissions, in accordance with one or more of the techniques described herein. If all scripts have been entered and verified, they can be marked as DONE to remove them from the queue. Click on DONE, answer OK to the ‘Are You Sure’ question, and the admit may be removed from the queue.

Click on the drop down to view that status of each Rx for the patient. Use the FILL VERIFY button to advance directly to the Fill Verify Queue, if wanted. They can also be verified directly from the Fill Verify Queue. Note: they may be identified as LTC in the Script Type with the name of the facility.

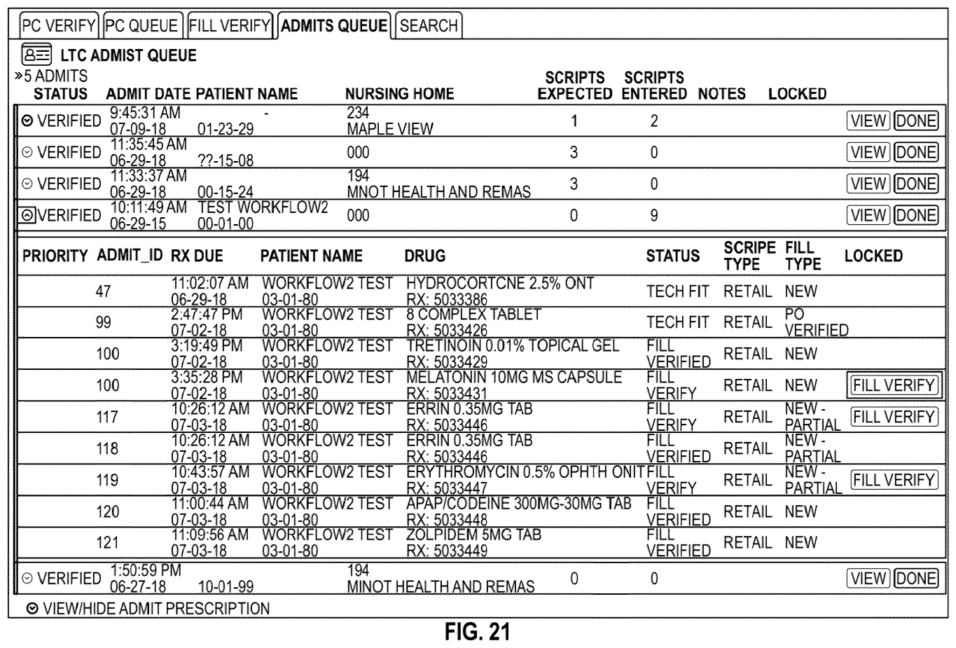

FIG. 21 is an example portion of a graphical user interface showing an example prescription fill list for long term care admissions with multiple prescriptions in a single order, in accordance with one or more of the techniques described herein. To Search for a specific prescription or patient, click on the Search Tab and enter the data fields to search by. In this example, the prescription number was entered.

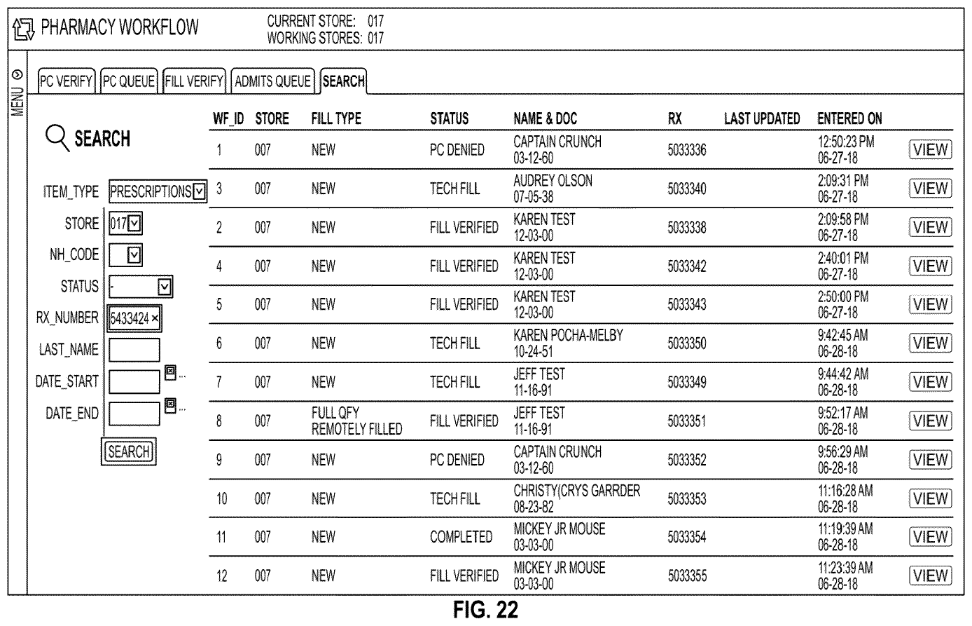

FIG. 22 is an example portion of a graphical user interface showing an example search page, in accordance with one or more of the techniques described herein. The prescription information may display along with the interactions, if any, and the Workflow History of the fill.

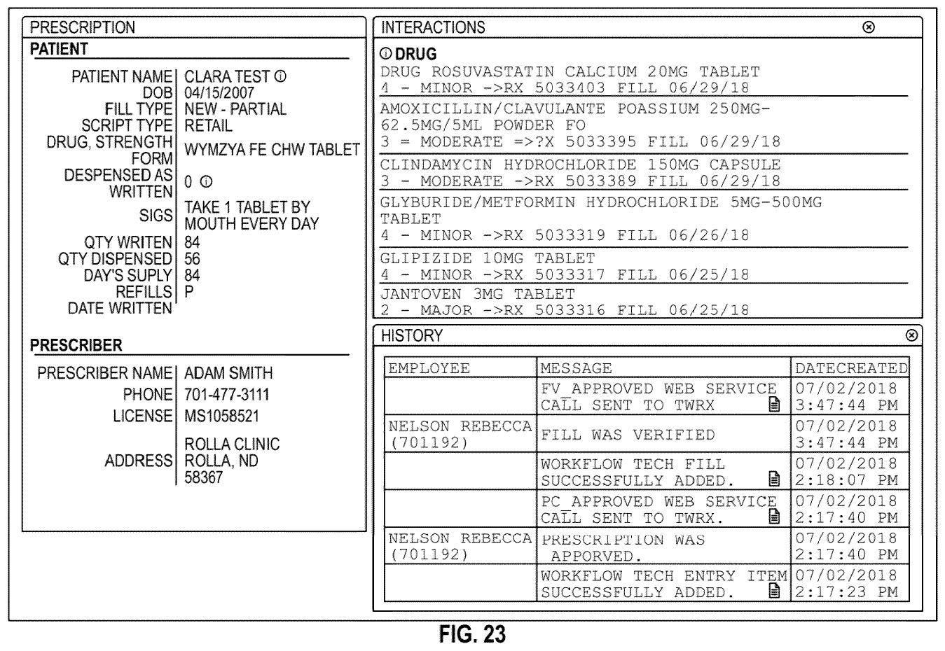

FIG. 23 is an example portion of a graphical user interface showing an example patient workflow history, in accordance with one or more of the techniques described herein. Once a prescription has been initially checked by the originating store, it proceeds in the process to the “Specialty” tab. The queue displays a status to identify which stage in processing that script is in (Tech Check vs. Pharmacist Check). The script undergoes an initial screening by a specially trained pharmacy technician. After that screening, one of the specialty pharmacists may perform the final review of the prescription before it is released for further processing, such as financial assistance or prior authorization resolution. These steps are conducted by the specialty pharmacy team prior to dispensing the medication.

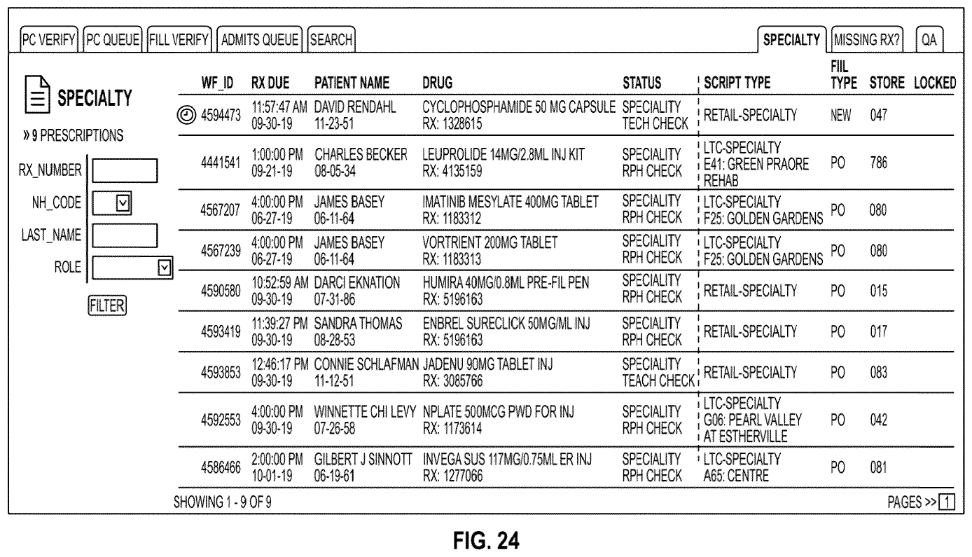

FIG. 24 is an example portion of a graphical user interface showing an example specialty prescription list, in accordance with one or more of the techniques described herein. Within a specific script, the system has created a mechanism for the local store to communicate to the specialty pharmacy team. This information is displayed in the “Specialty Coversheet Info” section of the script.

If the prescription is for an LTC patient, the system has embedded a form for the pharmacist to fax the facility for a hold order. This form may prepopulate with the relevant patient information. Access the Quality Assurance (QA) Queue by clicking on the QA tab in the upper right hand corner of the Workflow screen. The QA Queue is laid out in the same manner as the PC Queue. To complete a QA, click on the prescription.

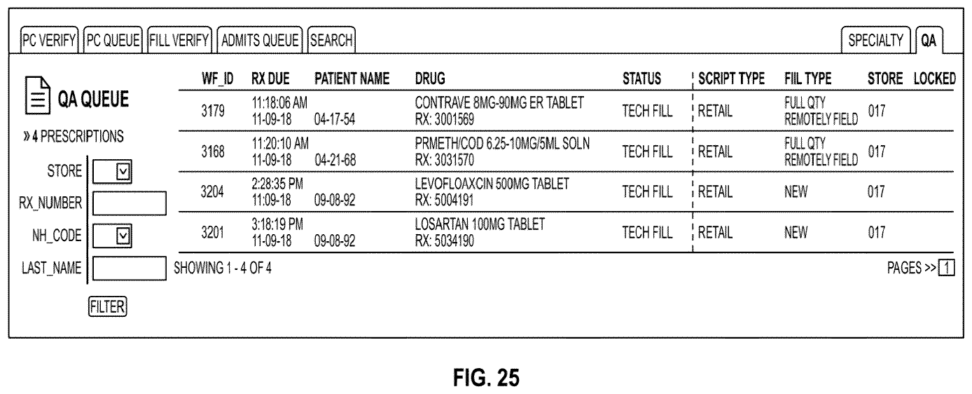

FIG. 25 is an example portion of a graphical user interface showing an example quality assurance queue, in accordance with one or more of the techniques described herein. The Clinical queue may house all of the clinical interventions generated using the artificial intelligence model.

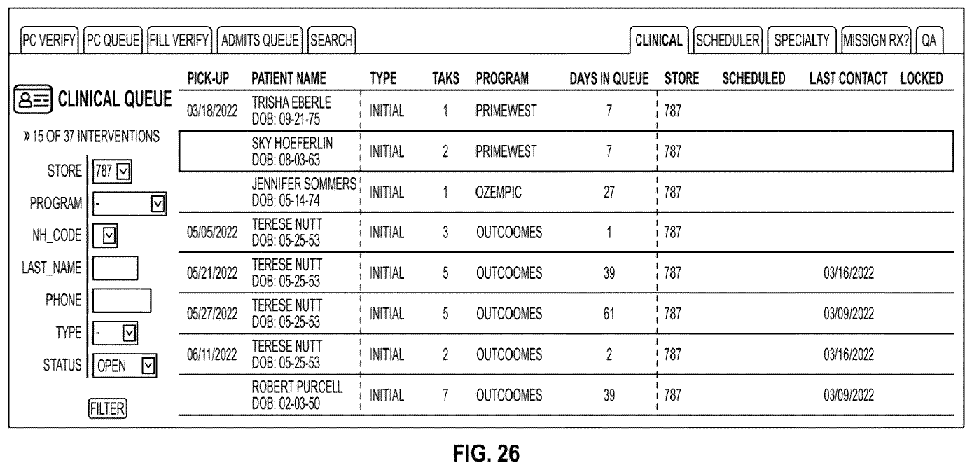

FIG. 26 is an example portion of a graphical user interface showing an example clinical queue, in accordance with one or more of the techniques described herein.

Within each patient, clinical conversations may be presented that the pharmacists can discuss with the patient.

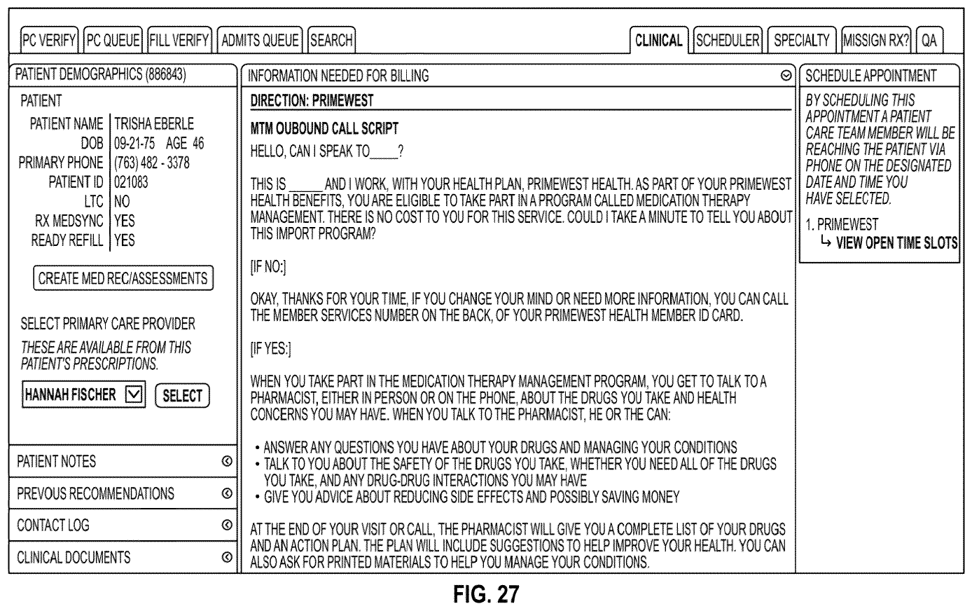

FIG. 27 is an example portion of a graphical user interface showing an example clinical conversation page, in accordance with one or more of the techniques described herein. Workload balancing allows for a pharmacist in one pharmacy to complete PC CHECK in another pharmacy. The pharmacies where a pharmacist can perform workload balancing may automatically be selected for them when they log in. They may be assigned based on states the pharmacist is licensed in.

In the PC CHECK queue, all prescriptions across the pharmacy group assigned to the individual pharmacist may display and are scripts that may need to be PC Verified. These prescriptions are in addition to the pharmacy's local prescriptions that may need PC Check completed for controlled substances, long term care, and prescriptions with marked with a clinical hold. Prescriptions being verified by another pharmacist may display in the queue as locked with the other pharmacist's information in the Locked column.

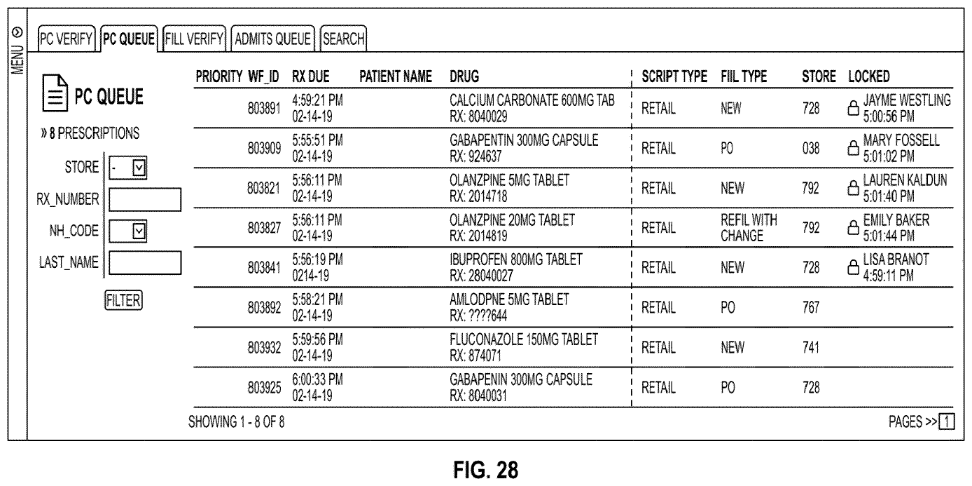

FIG. 28 is an example portion of a graphical user interface showing an example prescription fill queue with multiple prescriptions being locked, in accordance with one or more of the techniques described herein. The expectation is for every pharmacist to verify prescriptions as the come into the queue without picking and choosing. Using PC VERIFY may auto-feed prescriptions to the pharmacist to verify.

The system may also be configured to output important practice reminders, including: Be a good steward of the prescriptions the user is working on. If the user needs to step away from the queue, be sure a prescription is not left opened. This may cause the order to appear locked to any other pharmacist trying to verify; and be especially mindful if the prescription is marked as a Waiter. Complete the verification or click out of the prescription before stepping away.

If there is a prescription open and the browser is closed, the script may stay locked to the user. If the user has a prescription that the user needs to fill and it is locked by another pharmacist for more than 5 minutes, complete a Help Desk Ticket with the Rx # so Tech Support can free up the prescription.

If the user needs to view only prescriptions in the user's pharmacy, the view can be filtered. For example, if the user is looking for a specific prescription, or may need to get an LTC order pushed through prior to delivery, etc. This function should be used sparingly.

Workload balancing also applies to the QA Queue in addition to the PC Check queue. The system described herein may include a number of unique features, including: Auto-feed functionality of next available Rx in queue; Highlighting on the screen—safety and quality control; LTC Card Imaging; Uniqueness of the GUI; Locking of prescriptions, what store they are at, which individual; Waiters notated by the clock icon; Hold—think of it as a virtual stack of problem prescriptions; Co-mingling of both Retail and LTC prescriptions; Future Roadmap Items to Patent; Integration with Patient Safety Evaluation Systems (PSES), which includes tracking errors and near misses in the Patient Event Reporting Tool; and in states that allow technician-product-verification (TPV or TCT), the system can perform this step.

The system may have a will-call management tab which in addition to assisting in locating prescriptions in will-call, may also display consultation notes as well as interventions from the artificial intelligence model. The system may capture images during the filling process to be used for remote FV, which may enable the system to workload balance FV. Additionally, this may help streamline the process for the tele-pharmacies.

It has been recognized that as healthcare shifts to a value-based care model that there may continue to be an ever pressing need to engage with patients to improve outcomes. Based on these needs, several clinical initiatives were developed and implemented in the pharmacies. As the volume of clinical services being performed increased, several challenges were identified by the pharmacists: Time to conduct interventions (e.g., how can a pharmacist step away from their traditional dispensing responsibilities to conduct these interventions); Patient identification (e.g., what conversation does the pharmacist need to have with a given patient); and Multiple platforms (e.g., once the pharmacist completes a conversation, the pharmacist has to log into a variety of different platforms to document the conversation to receive payment, or once in the applicable platform, the pharmacist often has to perform manual data entry in order to get the intervention billed out).

In order to be successful in delivering these clinical services, the system provided solutions to these issues. The system may: Find a way to manage the workflow so the pharmacist had time to conduct the conversation; Easily identify patients in need of an encounter; and Document and bill for those conversations in an efficient manner.

There is not a platform available that could address all three of these challenges. The commercially available clinical platforms were not well designed to fit within a pharmacy dispensing application and documentation processes tended to be overly complicated. The commercially available dispensing applications did not meet the unique business needs and had limited clinical functionality.

As a result, the techniques described herein include pharmacy dispensing workflow application with an embedded Clinical Platform powered by an artificial intelligence model described herein. The application enables workload balancing the dispensing activities across multiple pharmacies, so that the pharmacist has time to step away from the dispensing functions and complete the clinical interventions. The platform then clearly identifies which patients may need a specific encounter, so that the entire pharmacy team has visibility to ensure the billable opportunity is not missed. The platform allows the pharmacists to complete the encounter within the same application they use for prescription fulfillment, streamlining the documentation process. It also enables the whole pharmacy team to deliver interventions to the patients they serve in such a way that is not possible with traditional workflow systems available today.

In a pharmacy workflow, the disclosure describes a process for completing patient care services that is supported by central fill facilities, which remove workload from the local stores, providing time for the pharmacists to intervene. The system then leverages an appointment model, which makes it possible for a patient to make just one trip to the pharmacy each month. These monthly visits enable a longitudinal approach to patient care that addresses patients' and pharmacist's prioritized concerns, goals, and planned interventions. The recurrent nature of these visits allows for ongoing revenue generation.

During that visit, the point of sale system alerts the clerk that there is an opportunity for this patient. The pharmacist is then able to intervene with the patient, making it easy to complete these interventions “on the fly” as patients use the pharmacy for traditional prescription needs. This improves store level completion rates, as well as reduces the number of cases that need to be conducted over the phone. The end result is the ability to take a traditional transaction and add on a billable event, such as an immunization, consultation, or drug administration.

The Clinical Platform is powered by a proprietary data analysis process. The techniques described herein employ analytics and proprietary algorithms to identify profit driving clinical interventions based on patient data. These interventions are based on current clinical guidelines for care and may involve recommendations relating to medication interactions, therapeutic contraindications, or management of a particular disease state. Once generated, these opportunities may be displayed in the Clinical Platform. Examples include: Screening for potential drug therapy problems due to therapeutic duplication; Drug-disease contraindications; Drug-drug interactions, including interaction involving over-the-counter medications; Incorrect drug dosage or duration of drug treatment; Drug-allergy interactions; Clinical abuse/misuse; Inappropriate medications based on clinical criteria; Overutilization or underutilization; Medication cost; Medication Recalls; Medication Shortages; Non-adherence; and Gaps in therapy such as missing medications or immunizations.

The implementation of the artificial intelligence model has yielded dramatic improvements in the ability to complete Comprehensive Medication/Medical Reviews (CMRs) more efficiently and with greater quality and consistency. Prior to implementation, pharmacists would spend about an hour from start to finish when conducting a CMR. Steps such as pre-working the case to develop recommendations, talking to the patient, and then documenting the encounter all took too much time. This made the business model unprofitable. The artificial intelligence model eliminates the need to pre-work the case and presenting the intervention in the Clinical Platform streamlines the documentation. The end result is that the CMRs now take between 5 and 20 minutes to complete. These efficiencies have made it viable to complete CMR's as part of workflow and may be a catalyst for significant growth in clinical revenues.

Additionally, the quality of the CMRs may also improve. Traditional patient care models that relied on a human's ability to identify an issue with the patient and then properly assign a solution result in wide variability in the care that patients receive from provider to provider. The artificial intelligence model may eliminate this phenomenon by consistently presenting clinically relevant and evidence-based recommendations. The end result is that any pharmacist using the system becomes a top-tier clinician, regardless of their individual knowledge.

The artificial intelligence model has utility beyond a clinical recommendation engine. It also serves as an intermediary, translating a variety of data types, aggregating interventions to be completed and exchanging information pertaining to patient care.

In addition, every medication therapy problem (MTP) identified by the artificial intelligence model as well as the intervention to address the problem are tied back to a SNOMED CT (Systematized Nomenclature of Medicine Clinical Terms) code for documentation and billing. SNOMED CT is the most comprehensive multilingual clinical healthcare terminology and is the international medical terminology standard.

Using SNOMED CT within the artificial intelligence model and the Clinical Platform enable the techniques described herein to communicate in a common language with other health care providers, thus increasing the quality of patient care across many different provider specialties. The intent for using SNOMED CT codes was to provide efficiencies in analyzing data to determine the impact that the innovative patient care approach is having on patients. In addition, using SNOMED CT codes enables implementation of the Pharmacist eCare Plan within the platform which serves as a standardized, interoperable document for exchange of prioritized medication-related activities, plans and goals for patients.

The artificial intelligence model can be used in a variety of ways. Pharmacies are able to create their own initiatives using the Clinical Admin Tool. This allows them to customize their programs and interventions based on their organization's priorities. The techniques described herein have used this platform in a variety of ways.

A first way to use the Clinical Platform is for Medication Therapy Management (MTM). The artificial intelligence model identifies interventions for pharmacists to intervene as part of the MTM process. The tool uses proprietary algorithms to identify Medication Therapy Problems (MTPs) as well as potential resolutions and a list of patient-facing recommendations to be included in the standard patient take away as well as a complete medication list along with the directions for use as part of the output for each patient. This significantly reduces the steps and time needed for documentation. It also includes provider facing pre-written evidence-based communication to address the MTPs with the provider.

The system has used this capability in several MTM settings, such as: Comprehensive medication reviews (CMRs); Targeted medication reviews (TMRs); Prospective Medication Reviews (PMRs); Medication Reconciliation; Discharge Counseling; and Immunization and Medication Administration.

The artificial intelligence model helps target patients that may be in need of immunizations. Research has shown that patients are much more likely to accept a vaccine recommendation when they are properly targeted. The system interfaces with state vaccination registries to identify patients with missing vaccines, and then compare those opportunities against patient's disease states. This allows the system to identify patients who are most likely to accept the recommendation and queue up only those conversations. This makes for a more efficient use of the pharmacist's time, while also improving acceptance rates.

A second way to use the Clinical Platform is for Point of Care Testing. Pharmacists play a key role in disease state management. For patients with existing medical conditions, the artificial intelligence model identifies patients that are due for a checkup, such as an A1c or blood pressure check. Additionally, patients that are at elevated risk for certain diseases are identified so the pharmacist can screen them, and if necessary, refer the patient to their primary care provider.

A third way to use the Clinical Platform is for DIR Mitigation. The artificial intelligence model identifies non-adherent patients and presents opportunities for pharmacists to intervene to ensure the patient takes their medication as prescribed. Pharmacists can address the root cause of the non-adherence through patient education and behavior modification through motivational interviewing.

A fourth way to use the Clinical Platform is for Opioid Interventions. Pharmacists are on the front lines of the opioid abuse epidemic and the artificial intelligence model identifies key interventions including opportunities for education, naloxone dispensing, and more. The goal is to lower the accidental overdose of opioids by delivering evidence based targeted interventions.

A fifth way to use the Clinical Platform is for Pharma Sponsored Enhanced Counseling Programs. As many as 30% of people do not pick up their prescribed medications. And when they do, as many as 30% of them stop taking their medicine within 30 days. While medication adherence (or lack thereof) has significant negative impacts on patient health outcomes, it also may have a profound impact on the revenue and perceived value of a pharmaceutical company's branded medications. According to industry research, the U.S. pharmaceutical industry loses an estimated $188 billion each year as a result of medication non-adherence. According to a 2017 survey, brand managers desire to implement robust medication adherence programs, but are all too often faced with organizational barriers, such as being unable to develop a clear ROI (45 percent). The techniques described herein have been able to help pharma partner brand managers break through those barriers with proven ROI which has resulted in renewal year over year of Statements of Work that address the roadblocks for patients to be adherent to their specific products. The artificial intelligence model running in the background identifies the need and opportunity to intervene and those interventions may be presented in the pharmacist workflow. Interventions may include first and second fill counseling, injection training, administration of injectable medications and adherence rescue calls.

The National Committee for Quality Assurance's (NCQA) Health Effectiveness Data Information Set (HEDIS) is a tool utilized by more than 90% of US health plans. It is comprised of 81 measures across 5 domains of care. HEDIS measures address important factors such as Asthma Medication Use, Persistence of Beta-Blocker Treatment after a Heart Attack, Controlling High Blood Pressure, Comprehensive Diabetes Care, Breast Cancer Screening and Antidepressant Medication Management. In addition to the artificial intelligence model's ability to identify opportunities to intervene to improve HEDIS measures it can also accept data feeds from health plans that are used to flag patients that may need interventions to address a HEDIS measure.

Another way to use the Clinical Platform is for Chronic Care Management Interventions. Chronic disease is responsible for about 75% of the nation's aggregate health care spending, as well as 96% of Medicare spending and 86% of Medicaid spending. About 45% of people in the United States suffer from at least 1 chronic disease, and more than two-thirds of all deaths are caused by 1 or more of 5 chronic diseases: cancer, chronic obstructive pulmonary disease, diabetes, heart disease, and stroke, according to the National Association of Chronic Disease Directors. Medication use is the predominant intervention for chronic disease which has presented a significant opportunity for the artificial intelligence model to identify opportunities (e.g. age appropriate dosing, drug interactions, gaps in therapy) to improve a patient's health outcomes. It also has created opportunities to work with health plans on new payment models and new revenue streams tied to the ability to impact the outcomes of patients.

Another way to use the Clinical Platform is for Health Plan, Employer Based Programs, and Department of Health Opportunities-Value Based Care. These entities have recognized that the techniques described herein have expanded the role of pharmacists to deliver clinical interventions in workflow and that have opened up opportunities for computing devices to do medical billing despite not having provider status. Examples of Medical Billing opportunities that computing devices have been allowed to complete include Point of Care Lab Testing (e.g. CLIA Waived Tests such as an A1C), Diabetes Self-Management Education (DSME), Pre-Diabetes Screenings, Immunizations, Medication Administration (i.e. injections) Opioid Use Management and Comprehensive Medications Reviews. In addition to these opportunities the techniques described herein also engage in Medication Reconciliation, Transitional Care Management and Care Coordination.

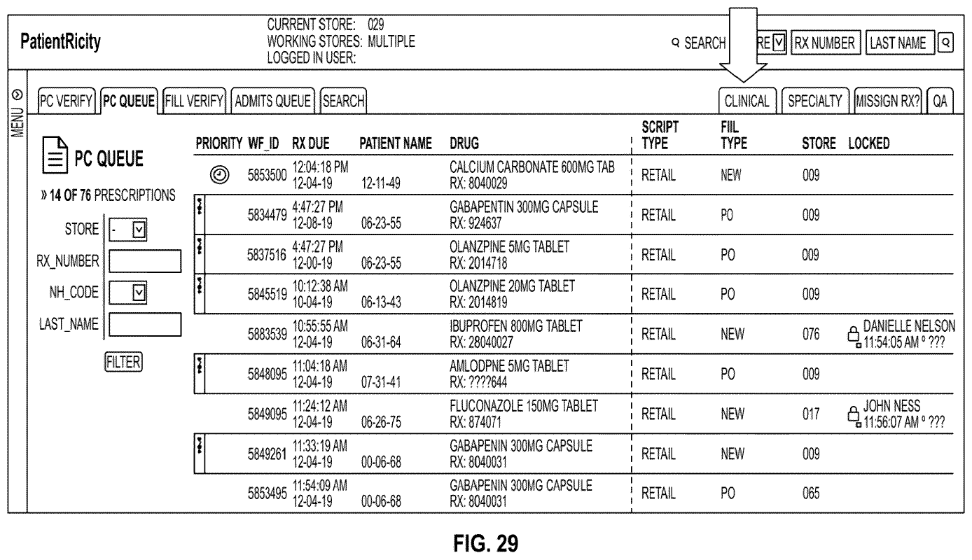

FIG. 29 is an example portion of a graphical user interface showing an example prescription list with a tab to access a clinical platform, in accordance with one or more of the techniques described herein. The Clinical Platform is accessed through a tab on the main page of the application in the upper right-hand corner.

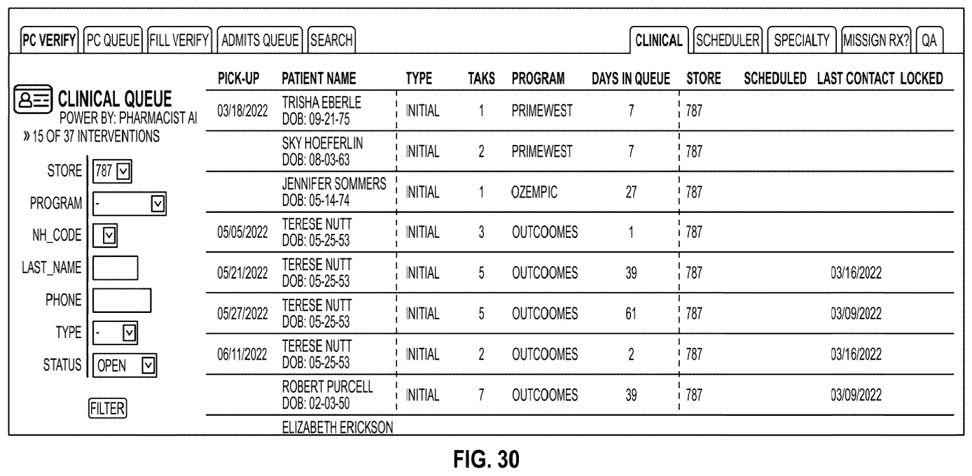

FIG. 30 is an example portion of a graphical user interface showing an example clinical platform, in accordance with one or more of the techniques described herein. The clinical queue displays all patients that have open recommendations which may need to be addressed by the pharmacist. Patients are sorted by the due date of the pending recommendation, which may align with an upcoming pickup date based on prescription fill data. This prioritizes the queue for the staff, so that opportunities may not be missed, as well as taking away the question of what should be done first.

The left-hand side of the screen displays the number of recommendations in the queue. The queue can be sorted by any of the methods displayed: Program, Patient Last Name, Phone Number, Type of Recommendations, or Status (Open or Completed Today). Select or enter the method to sort by, then click on FILTER. Searching by patient name may assist the pharmacist in efficiently locating the recommendation when the patient is in the pharmacy.

The columns on the main page may display the following: Pick-Up: the anticipated date the patient may be in the pharmacy next to pick up a prescription; Patient Name: and date of birth; Type: the type of recommendation; Tasks: the number of recommendations for the patient; Program: the clinical program the recommendation originated from; Days In Queue: the numbers of days the intervention has been in queue; Store: home store number of the patient; Scheduled: indicates if the patient has a scheduled appointment with qualified personnel for selected program; Last Contract: indicates the last day the patient was contacted; Locked: Displays the pharmacist's name who is currently working on the patient. To open a patient record, click on the applicable line. The system may have a built in scheduling system. This allows the user to schedule with a specialist for a particular encounter or referral. The scheduling system may include an admin area for setting up qualified professionals schedules and programs. Users may view their schedule and mark the status of the encounter. The system may send automated calls to patient the day prior to their appointment and may send calls for missed appointments.

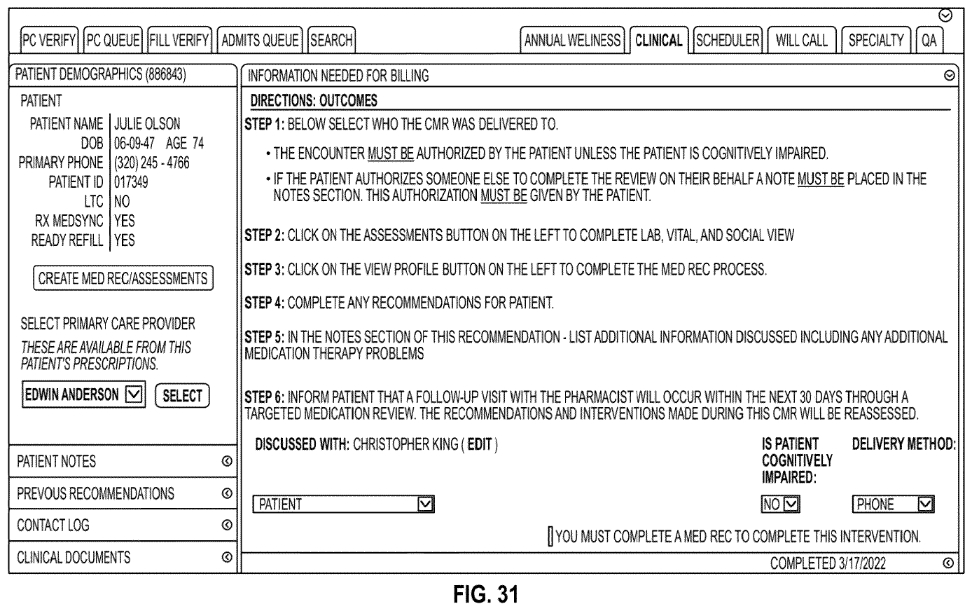

FIG. 31 is an example portion of a graphical user interface showing an example of information usable during patient conversations, in accordance with one or more of the techniques described herein. The information on the left hand side of the screen may display the same information for all recommendation types and programs. It displays relevant information that the pharmacist can use during their conversation with the patient. It also displays the patient's phone number which makes it easy for the pharmacist to call the patient to conduct the intervention over the phone.

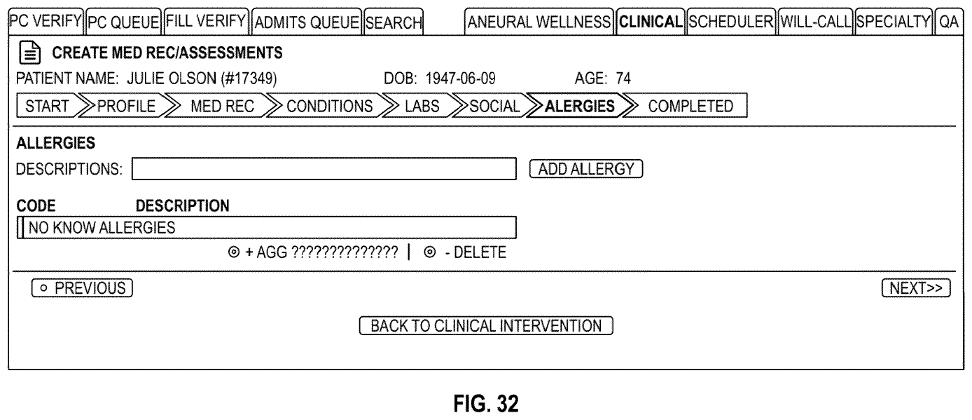

FIG. 32 is an example portion of a graphical user interface showing an example allergy information page, in accordance with one or more of the techniques described herein.

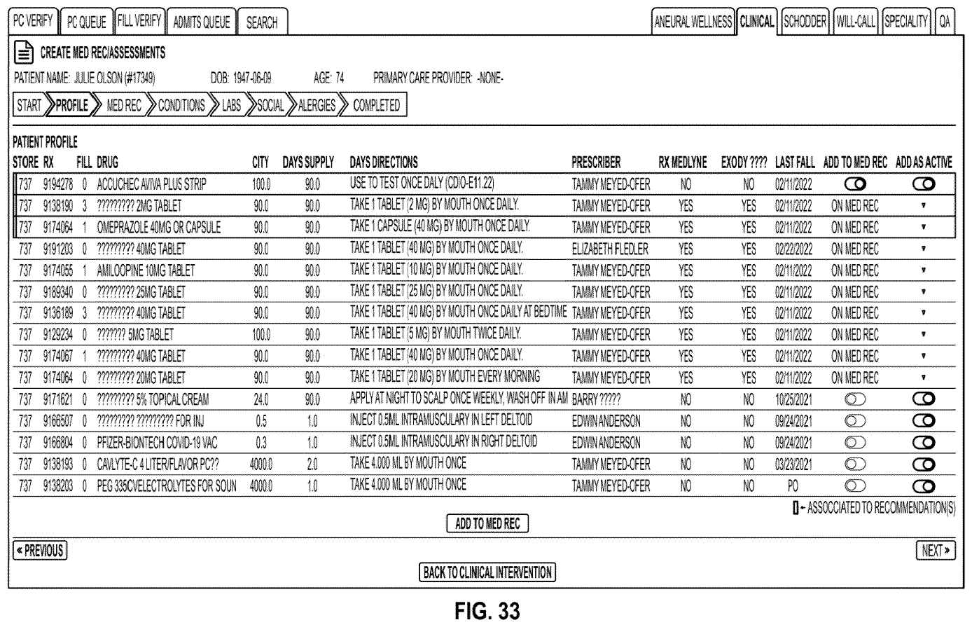

FIG. 33 is an example portion of a graphical user interface showing an example patient profile, in accordance with one or more of the techniques described herein. The complete medication profile may display. Use the scroll bar to move through the profile.

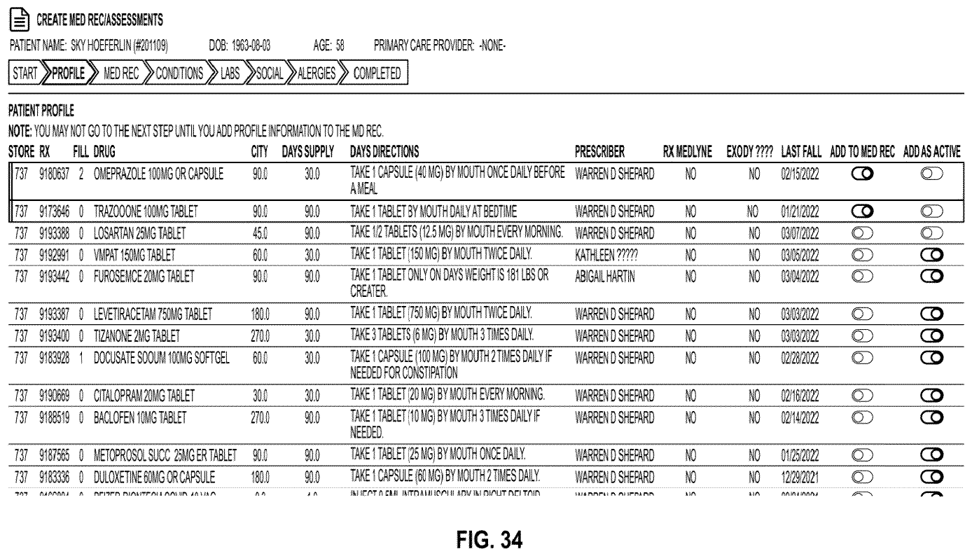

FIG. 34 is an example portion of a graphical user interface showing an example patient profile with inactive medications, in accordance with one or more of the techniques described herein. Medications that are no longer active, such as a limited therapy antibiotic, can be inactivated by changing the ACTIVE button from YES to NO.

Medications that the patient may be receiving from another source (OTCs, herbal products, drugs filled at another pharmacy, etc.) can be added by clicking on ADD MEDICATION. Enter in the information and click on ADD. All fields should be completed. Click CLOSE WINDOW to return to the main clinical screen.

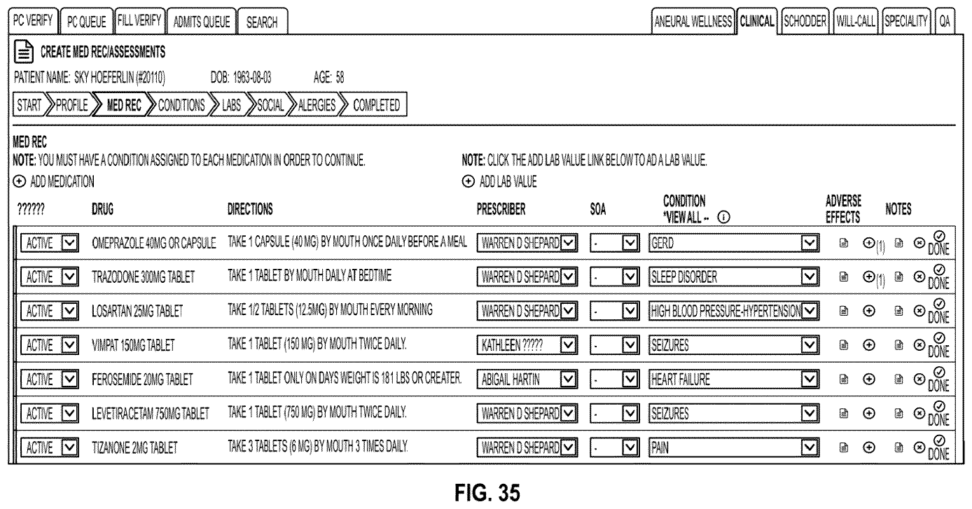

FIG. 35 is an example portion of a graphical user interface showing an example list of medications filled by third party pharmacists, in accordance with one or more of the techniques described herein. As the pharmacist is completing the medication reconciliation process, they are able to review the indications listed for the medications. They can add or modify indications based on their conversation with the patients.

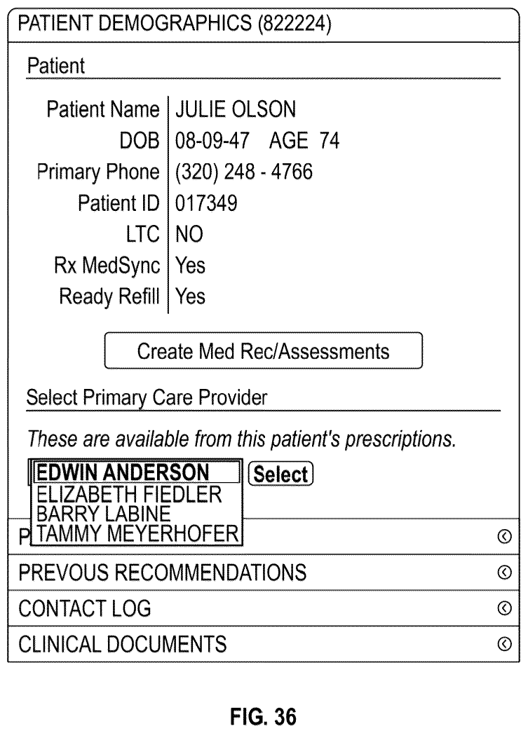

FIG. 36 is an example portion of a graphical user interface showing an example primary care provider page, in accordance with one or more of the techniques described herein. The primary care provider can be changed from the drop down list if needed.

The drop down may display all prescribers that have ordered prescriptions for the patient.

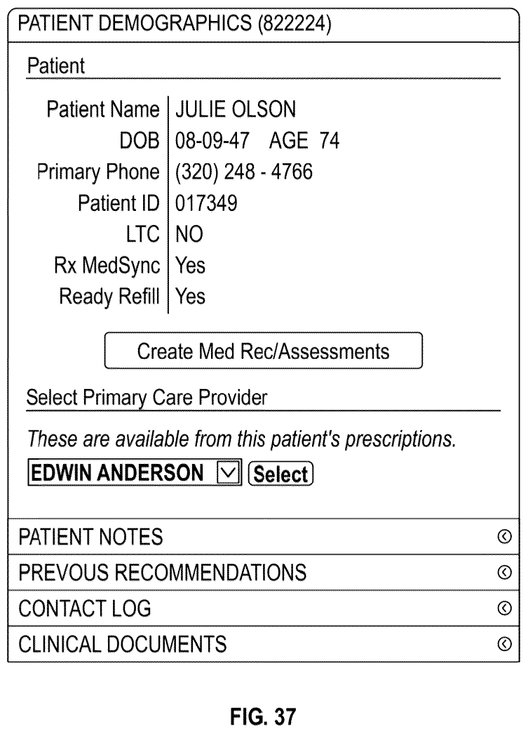

FIG. 37 is an example portion of a graphical user interface showing an example patient demographic page with expandable tiles, in accordance with one or more of the techniques described herein. There are four tiles available on the lower left side of the screen that can be expanded by clicking on the tile.

These tiles are:

Patient Notes—Specific things about the patient that are unrelated to a given program or recommendation. Examples would include “Patient is hard of hearing”, “Best time to reach patient is after 4 pm”, etc. The system is also able to note social history items, such as tobacco or alcohol use.

Previous Recommendations—Displays any recommendations that were previously generated. This is useful for the pharmacist to refer back to in order to see notes regarding previous recommendations, what course of action had been previously discussed, etc. Additionally, this allows a pharmacist to go back to a previous recommendation and update the status. For example, if the system faxed the doctor with a recommendation, and the doctor replied back to the system accepting the recommendation. The system would want to note that here for future reference.

Contact Log—Allows the store to document their attempts to contact the patient. This is useful in case the patient calls back, and the system may determine what the system called them about. Additionally, the time and date of the most recent call attempt may be displayed in the clinical queue. This allows the pharmacy team to monitor their progress, as well as using this information to target their outreach efforts to select patients.

Clinical Documents—This displays any previously generated Medication Action Plans, Patient Cover Letters, and Patient Medication Lists. This is useful in case the system may reprint a document for a patient, caregiver, or prescriber. It is also useful for the pharmacist in case they want to see what was previously discussed with the patient.

To complete recommendations, depending on the type of recommendation, and the program the recommendation is for, the patient screen may display different information. When accessing a Comprehensive Medication Review (CMR) recommendation, the information on the upper right hand side of the screen may display with the CMS requirements for billing a CMR. Use the drop down buttons to change any information: Discussed with: Select Patient, Caregiver, Prescriber, Other; Is Patient cognitively impaired: Select Yes or No; and Delivery Method: Select Face to Face, Phone or Video. Recommendations may display directly under the main window.

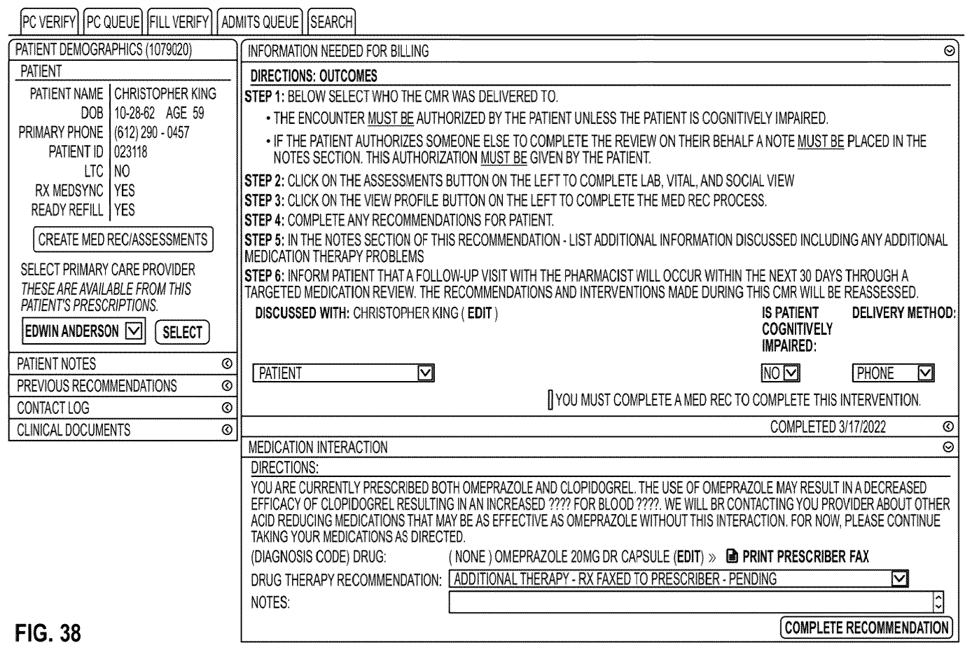

FIG. 38 is an example portion of a graphical user interface showing an example patient profile with substitute recommendations, in accordance with one or more of the techniques described herein. If the recommendation is collapsed and highlighted in green, that indicates the recommendation has been completed but can be updated. Click on the arrow to expand the recommendation.

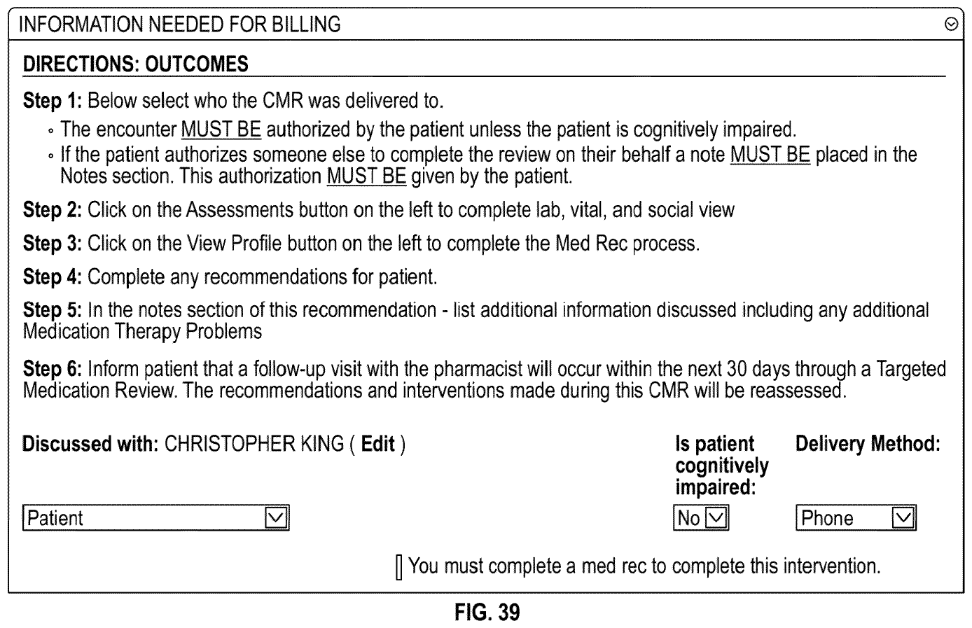

FIG. 39 is an example portion of a graphical user interface showing an example billing information page, in accordance with one or more of the techniques described herein. If the recommendation is collapsed, click on the arrow to expand the recommendation.

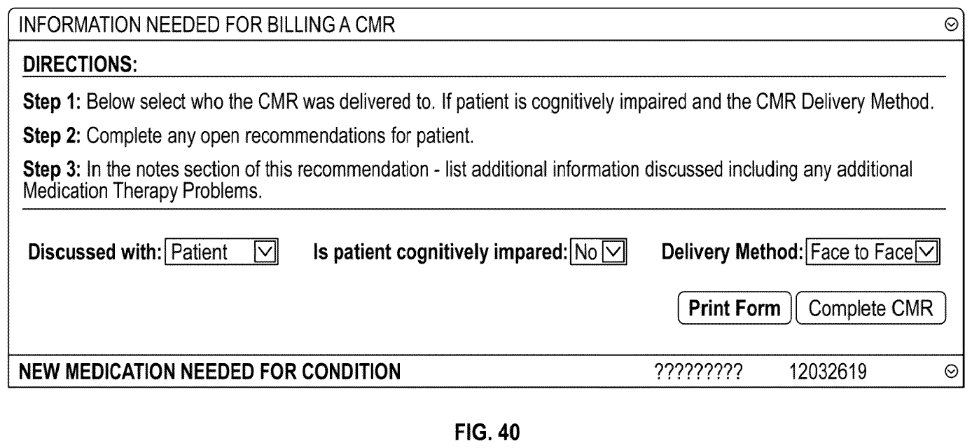

FIG. 40 is an example portion of a graphical user interface showing an example billing information page with a deferred recommendation, in accordance with one or more of the techniques described herein. For new recommendations, read through the Directions and select the CHANGE IN DOSE/FORM/INTERVAL RECOMMENDATION from the drop down options. NOTE: the directions and options may vary based on the condition, drug, etc.

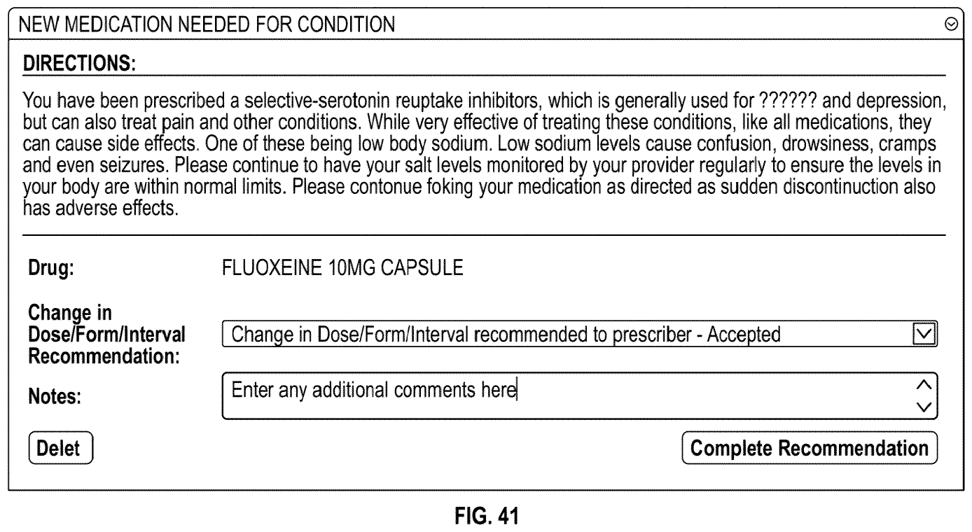

FIG. 41 is an example portion of a graphical user interface showing an example new medication recommendation, in accordance with one or more of the techniques described herein. Add any additional comments in the NOTES field and click on COMPLETE RECOMMENDATIONS.

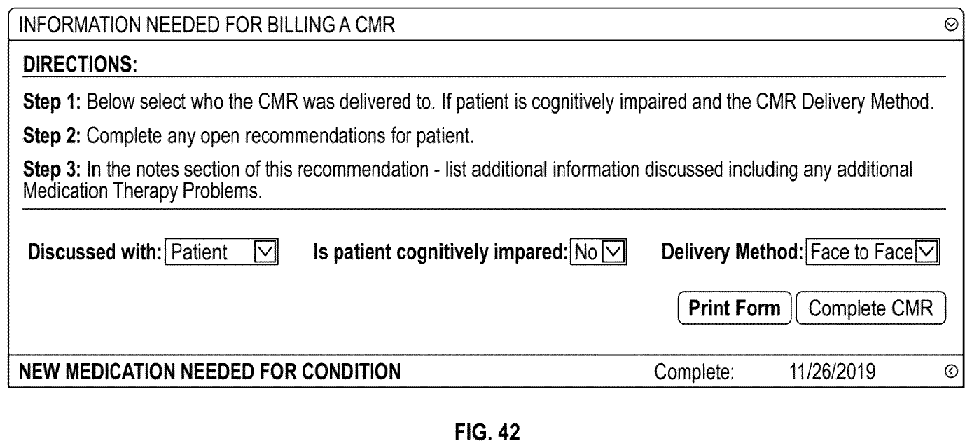

FIG. 42 is an example portion of a graphical user interface showing an example printing dialog box, in accordance with one or more of the techniques described herein. When the recommendation has been updated, click on PRINT FORM. A Cover Letter, Patient Medication List, Patient Action Plan may print with the CMR results which can be provided to the patient.

To complete and close out the encounter, click on COMPLETE. The patient is then removed from the queue. If the patient has multiple recommendations, they may display one after the other.

If the recommendation is a Targeted Clinical Intervention, the name may display in the blue header, such as DIABETES SELF CARE, ADHERENCE, Enhanced Counseling, Drug/Device Training etc. Select the appropriate Recommendation from the drop down, add any notes, and click COMPLETE RECOMMENDATION. Response options displayed in the intervention may vary based on the type of recommendation being made. For billing and reporting, the process may vary based on the type of billing.

For billing for clinical services, the application supports several data export options to facilitate the sharing records of the completed interventions between providers and payors. This data sharing allows providers to keep their electronic health record up to date with information and recommendations from the pharmacy. From a payer's perspective, the data allows them to record case completion and provide payment.

For Continuity of Care Document (CCD). each encounter allows for the generation of a CCD record. The CCD is a text delimited file intended to specify the encoding, structure, and semantics of a patient summary clinical document for exchange between healthcare professionals.

For 837P medical billing, the platform allows for claims to be medically billed electronically through an exchange or direct bill to plan. Billing uses the appropriate current procedural terminology (CPT) codes depending on the services performed. Medically billing pharmacy claims allows for the pharmacy to perform additional services resulting in improved overall health and additional revenue to the pharmacy.

For a pharmacist eCare plan, the pharmacist eCare plan is a standardized, interoperable document for exchange of prioritized medication-related activities, plans and goals for patients between pharmacists, prescribers, other healthcare professionals, as well as patients. Using this communication method, the application is able to transmit the encounters and recommendations directly into the prescribers EHR system, and they are able to accept or reject the recommendations, transmitting that information back to us. The application has a queue to house completed cases that may need to be addressed in order to be billed properly. This queue has two types of cases to address.

A first set of cases are cases that were billed electronically to a payer, which did not adjudicate properly. The majority of these claims may be identified as rejected due to the 835 file that is returned to the system after the 837P file is submitted. These claims may be in a “Rejected” status. Users may be able to work to address the issue and then resubmit the claim once resolved.

A second set of cases are cases that originated from an external clinical platform, where there is not an integration available to upload the completion data. In this scenario, a user may need to log into the external platform and transcribe the data into that platform. Once that has been completed, the user can mark the case as completed in the application. This ensures that the completion data in the system and the external system stay synchronized.

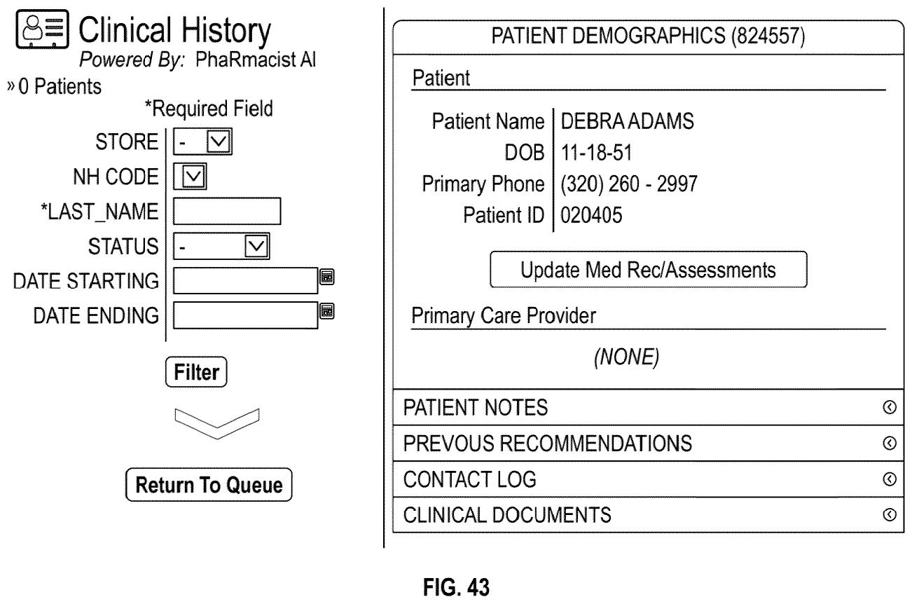

FIG. 43 is an example portion of a graphical user interface showing an example clinical history page, in accordance with one or more of the techniques described herein. After successful adjudication, payors have conducted audits to ensure the pharmacy has proper documentation to demonstrate that the encounter took place. Claims undergoing an audit are able to be viewed by searching for the patient within the Clinical History section of the platform. Once the patient in question is found, the user is able to review the history of their encounters. Data elements include patient demographics, patient notes, previous recommendations, contact log and clinical documents.

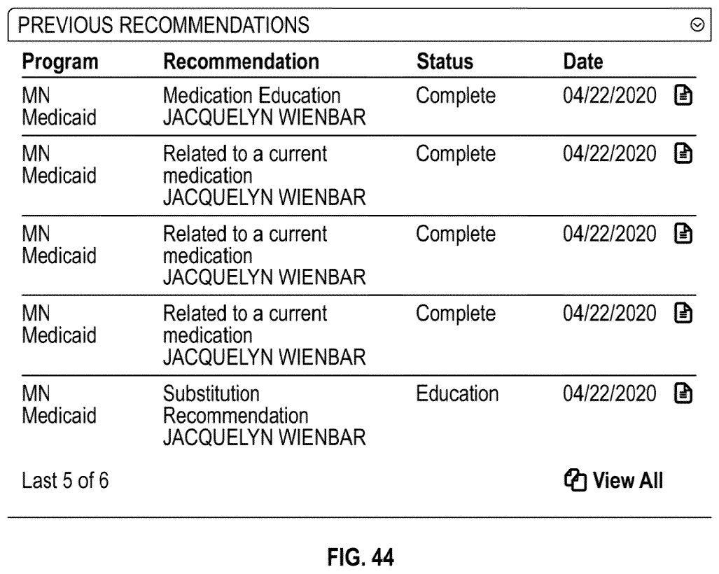

FIG. 44 is an example portion of a graphical user interface showing an example recommendation history page, in accordance with one or more of the techniques described herein.

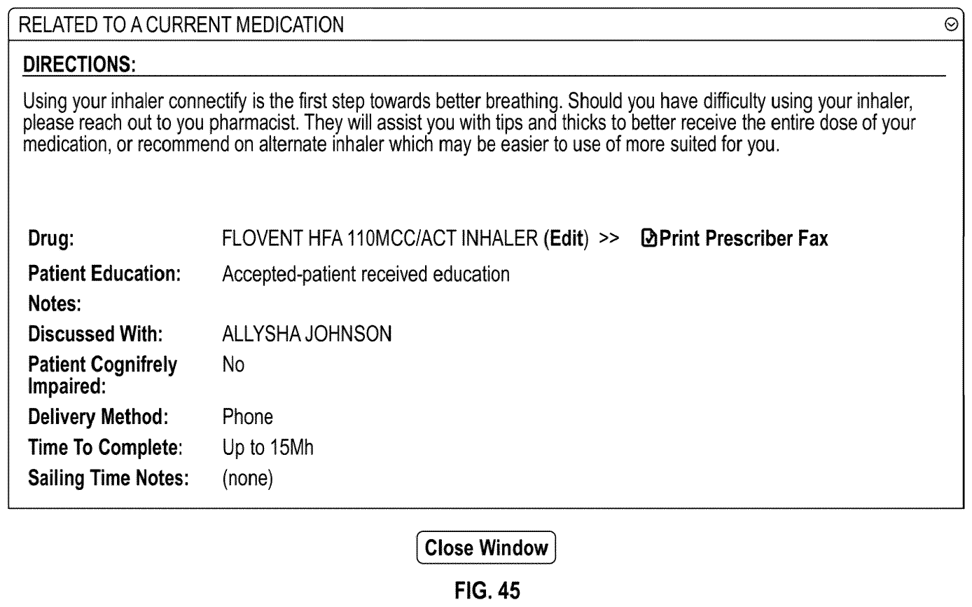

FIG. 45 is an example portion of a graphical user interface showing an example recommendation direction page, in accordance with one or more of the techniques described herein. The user is able to review the previous recommendations. The previous recommendations may display the program, recommendation, status and date of the encounter. The user is able to click into the recommendation to display the details within that recommendation; including notes, who the encounter was discussed with, the patient's cognitive status, delivery method, time to complete, and any applicable notes.

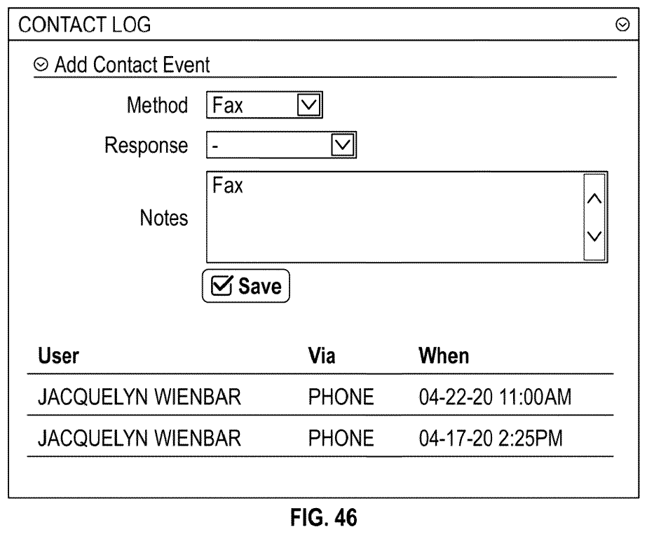

FIG. 46 is an example portion of a graphical user interface showing an example contact log, in accordance with one or more of the techniques described herein. The contact log allows the user to record fax, face to face, phone, email and video contact events. The timestamp is automatically recorded along with the user conducting the contact.

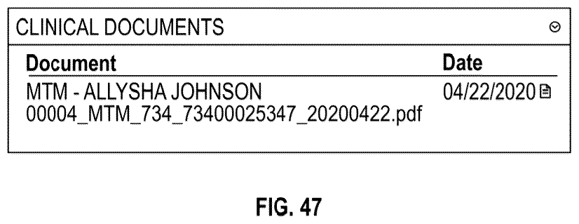

FIG. 47 is an example portion of a graphical user interface showing an example clinical document list, in accordance with one or more of the techniques described herein. The clinical documents section allows the user to review any clinical documents that were generated by the encounter such as the CMS standard patient takeaway, medication action plan, and medication list.

The irretrievability of the times, dates, paperwork, and intervention history all contribute to the creation of a system with a robust capability to defend against an audit. This is an important protection in order to protect revenues against recoupment by the payors.

The techniques described herein are able to generate automated and ad hoc reporting for use by store teams and their field managers in order to monitor and manage performance. These reports list the various programs a pharmacy is administering. The number of cases available, completed, and % completed are listed. The reports are delivered to the users via automated email distribution.

In addition to exception reporting that is pushed to users, a dashboard is available within the application. This dashboard displays a variety of customizable metrics, including clinical intervention completion status, pharmacist and technician dispensing metrics, inventory control, store task completion, payroll control, etc. The data comes from a data warehouse. External users can link up their own Data Warehouse data or use data from their dispensing application. The availability of data may impact the scope of the reporting available.

The Clinical Admin Tool allows non-technical users (for example pharmacy operations or clinical operations users, vs. information technology professionals), to create new programs, recommendations, disease states, and pharmacist facing answer sets without involving scarce IT resources.

In the event that a manufacturer, employer group, or insurance payer decides to initiate a new reimbursable program, super users within a given department (pharmacy ops or clinical ops) can set up a program that may address the patients in question with a customizable set of interventions.

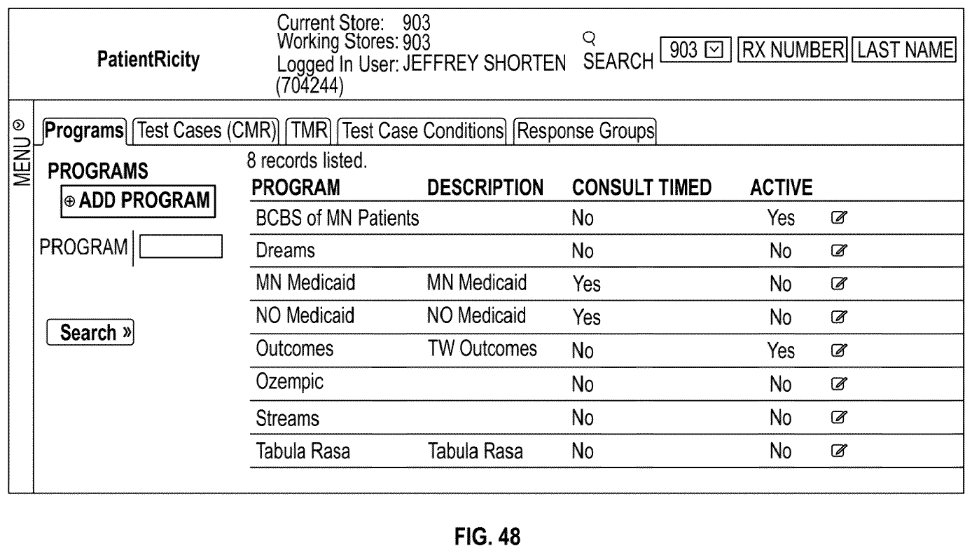

FIG. 48 is an example portion of a graphical user interface showing an example option to add a program, in accordance with one or more of the techniques described herein. The user may first click the “Add a Program” button:

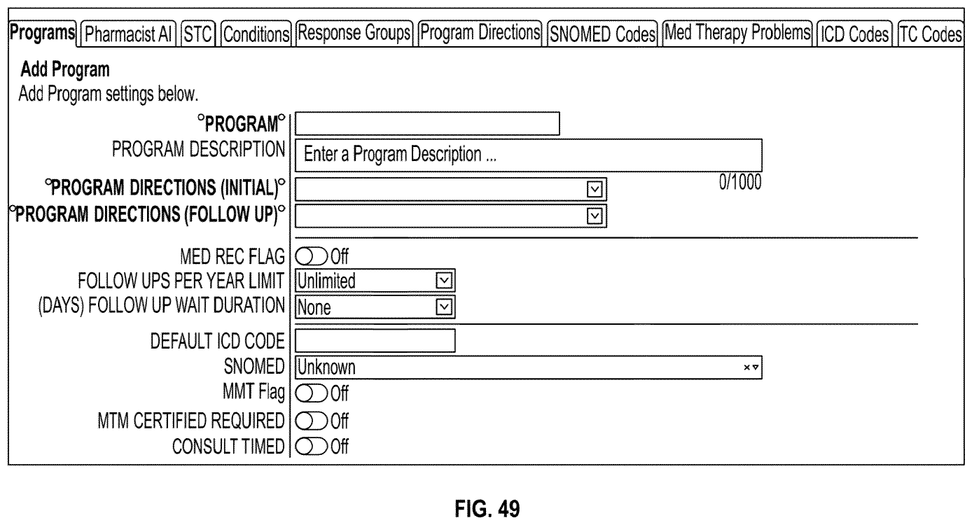

FIG. 49 is an example portion of a graphical user interface showing an example prompt for adding a program, in accordance with one or more of the techniques described herein. Within the Add Program functionality, the super user is allowed to customize the program details, including: Program Details; Program Name; Program Description; and a short definition of what the program is intended to do.

Program pharmacist guidelines provide instruction to the pharmacist on what they are supposed to do for a given patient. For example, if the program is sponsored by pharma to conduct first fill counseling, there would be instructions to the pharmacist letting them know that this is a new medication for this patient, and that they should be counseled on specific points outlined in this section. Follow Ups Per Year Limit allows the user to specify how many visits per year the payer is willing to pay for. Days Follow Up Wait Duration allows the user to set how often a subsequent intervention is queued into the Clinical Platform. For example, some payers may only pay for one intervention per month, so in order to maximize reimbursement, this field would be set to 30 days.

Some programs may require the pharmacist conducting the intervention to be “MTM Certified”. If this option is selected, the artificial intelligence model may access the “Credential Tracker” application to determine if a given pharmacist qualifies to work on this case. If they do not, then that case will not appear in their queue.

Some payers reimburse providers based on the time spent with the patient. These time allotments may be billed using CPT codes. If this option is selected, then cases completed in this program may track the time spent on the intervention and apply the correct CPT code for billing.

If the load balance option is checked, this means that other stores in the same workload balancing grouping would see these cases in their queue. This means that any available pharmacist could work on this case, rather than just being isolated to one given pharmacist at the store the patient goes to. The active option is a way to activate or deactivate a program if needed.

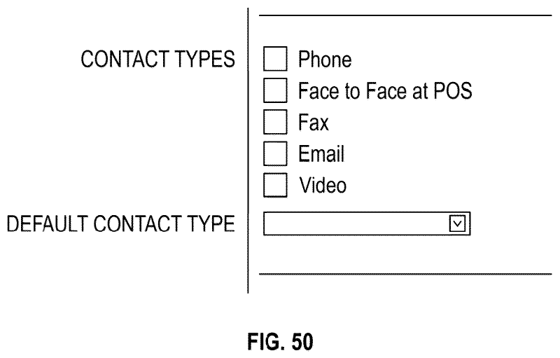

FIG. 50 is an example portion of a graphical user interface showing an example contact method selection, in accordance with one or more of the techniques described herein. Certain programs may only allow for specific communication types. Some insurance plans may only pay for face-to-face consultations. In that case, the system may not let a store complete the intervention through an incorrect method. Based on the types selected above, the contact default dropdown menu allows the user to set the default contact type that may be displayed for the pharmacists. Many programs are highly skewed toward one contact type, and this saves time by reducing clicks for the pharmacist when completing the intervention.

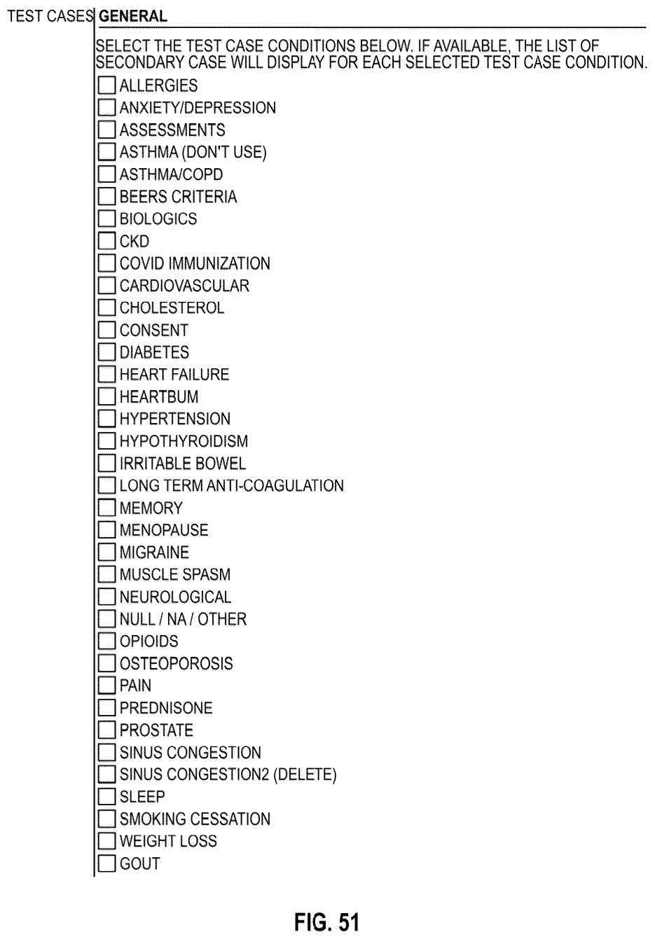

FIG. 51 is an example portion of a graphical user interface showing an example list of test cases in accordance with one or more of the techniques described herein.

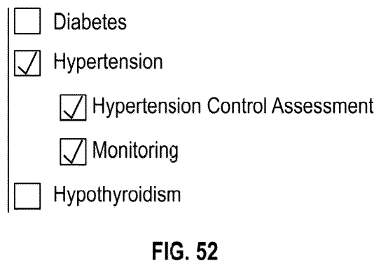

FIG. 52 is an example portion of a graphical user interface showing an example targeted intervention list, in accordance with one or more of the techniques described herein. The test case allows the user to select which types of interventions may trigger for a patient enrolled in this program. For example, if a payer wanted a program designed to manage their blood pressure patients, then the user would select only the hypertension option. Once selected, if there are targeted interventions created for that disease state, then the user has the option to select whichever specific ones the user would want to include in the program.

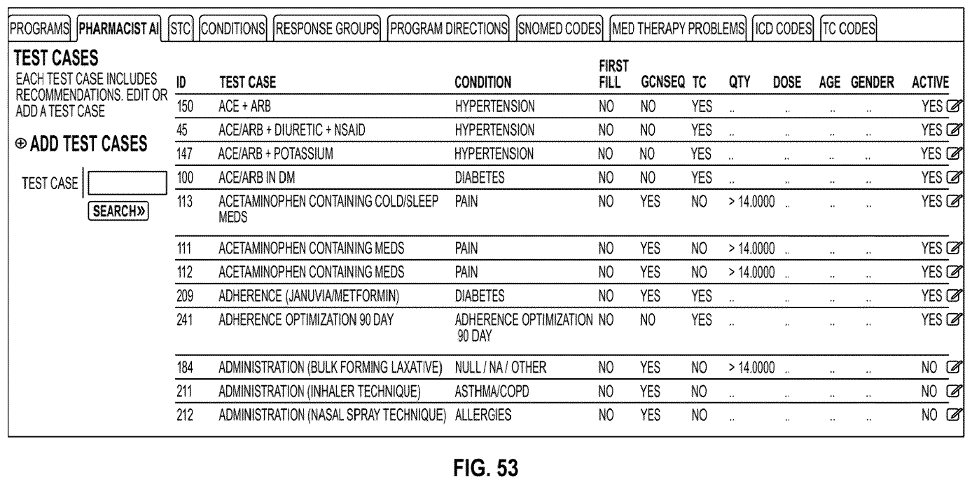

FIG. 53 is an example portion of a graphical user interface showing an example option to add a Pharmacist AI test case, in accordance with one or more of the techniques described herein. Recommendations that are displayed during an encounter are the result of a patient being flagged by a “test case”. If the patient fits the criteria designated within the test case, the associated recommendation may be presented. Users can add a Test Case by clicking the “Add Test Case” button on the left hand side of the Pharmacist AI tab.

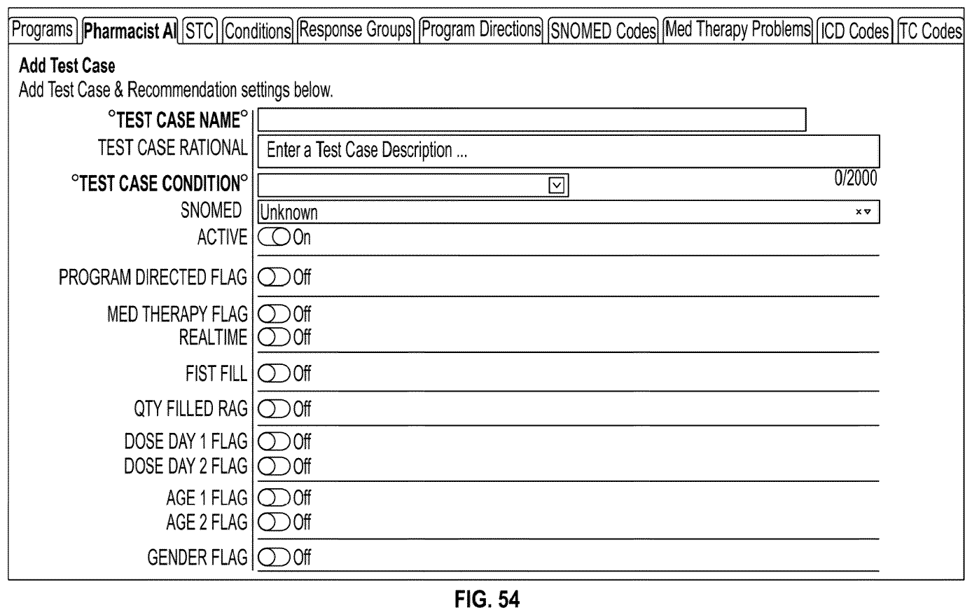

FIG. 54 is an example portion of a graphical user interface showing an example prompt to add a test case, in accordance with one or more of the techniques described herein. Once they are in the “Add Test Case” function, there are several fields that allow the user to set the conditions may be required to trigger this recommendation within the clinical queue for a patient.

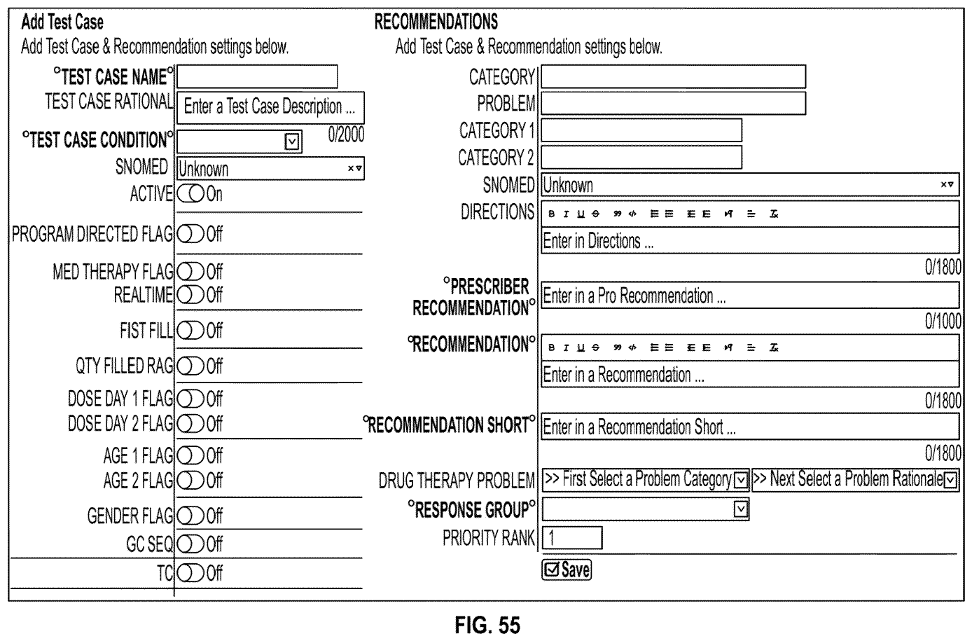

FIG. 55 is an example portion of a graphical user interface showing an example test case recommendation entry page, in accordance with one or more of the techniques described herein. The Test Case Name and Test Case Rational fields provide a short definition of what the test case is intended to look for and references clinical guidelines. The Test Case Condition field enables the user to select the disease state grouping that this test case should be tied to. The Active field provides a way to deactivate a test case if needed. The Program Directed Flag field provides that if this flag is set, then the test case may trigger automatically for any patients enrolled in a given program. No other test case criteria may be evaluated. For example, this could be used if an employer group wants all of their employees to have the pharmacists discuss a specific topic, such as seasonal flu shots.

The Realtime field provides that the test case may be evaluated in real time as a prescription is processed through the dispensing application. The First Fill field provides that the test case is designed to only apply to certain fill numbers, and the system may need to evaluate if the patient has been on this (or a similar) medication before. The Quantity Filled Flag field allows users to set thresholds that would cause the test case to trigger based on the amount of drug being dispensed. The Dose Day 1 & 2 Flag field allows users to set thresholds that would cause the test case to trigger based on daily doses of medication. The Age 1 & 2 Flag field allows users to set thresholds that would cause the test case to trigger based on a patient's age. The Gender Flag field allows users cause the test case to trigger based on a patient's gender.

The GCN Sequence field allows the user to create inclusion and exclusion criteria based on the medications a patient is on. The Therapeutic Code (TC) field allows the user to create inclusion and exclusion criteria based on the medications a patient is on. The Problem field provides a short summary of what the recommendations are addressing.

The Category field is a field to display header information on the user interface of the recommendation within the encounter. The Directions field provides instructions to the pharmacist to let them know what the intervention is about, why it is being conducted, what steps may be needed to be completed, etc. The Provider Recommendation field provides the prescriber facing language that would be sent if the pharmacist wishes to make a recommendation to them. Examples would be requests for a change in dose, information regarding a drug interaction, or a referral if the patient needs to be seen. The Recommendation field provides patient friendly language the pharmacist may read to the patient when conducting the intervention. The Recommendation Short field provides a shorter version of the patient facing recommendation. The number of characters is limited so that the recommendation fits on the CMS compliant patient take away document.

The Drug Therapy Problem field allows the user to set what appropriate Drug Therapy Problem the recommendation is classified as. This streamlines the delivery of CMRs that the pharmacist does not need to spend time trying to figure out what the program is classified as. This is in accordance with PQA MTM frameworks standard.

This framework is editable within the Clinical Admin area. The Response Group field provides is the set of options that the pharmacist may be able to choose from during the intervention within the application. For example, an intervention focused on administering a flu shot to a patient may use the following options:
Patient accepts—RPh to administer
Patent declines—Has appointment with other provider
Patient declines—Self-administers (if applicable)
Not applicable—Medication picked up by caregiver
Patient declines—Other

An intervention focused on a change in dose recommendation to the prescriber would have different options:
Change in Dose/Form/Interval recommended to Prescriber—Accepted
Change in Dose/Form/Interval recommended to Prescriber—Declined
Change in Dose/Form/Interval recommended to Prescriber—No response or pending
Patient Declined
Educated Patient—No further immediate action required
Not clinically relevant

The super user can select any response group they want. If there is not one that matches what they are trying to do, they can create one. This process may be detailed later in this document.

The Priority Rank field affects the order that the recommendations are presented if there are several recommendations active for a patient.

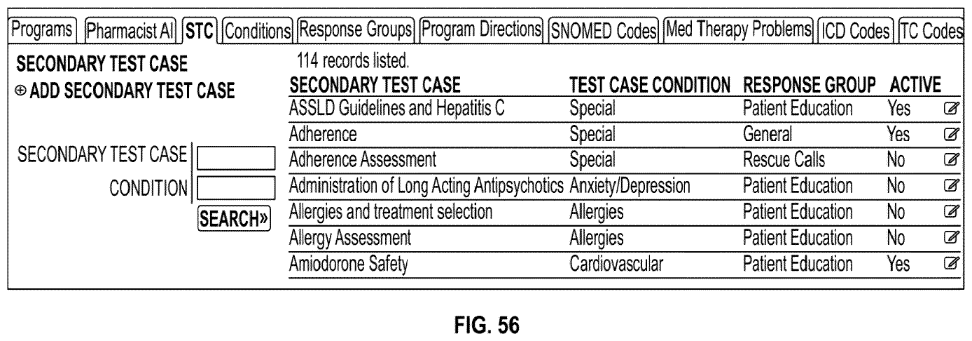

FIG. 56 is an example portion of a graphical user interface showing an example test case page with an option to add a Secondary Test Case, in accordance with one or more of the techniques described herein.

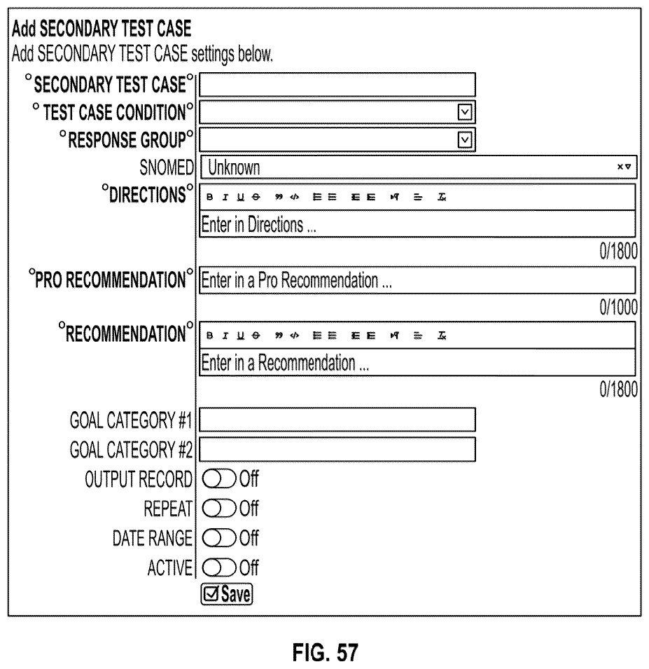

FIG. 57 is an example portion of a graphical user interface showing an example prompt to add a Secondary Test Case in accordance with one or more of the techniques described herein. Users can add a Secondary Test Case by clicking the “Add Secondary Test Case” button on the left hand side of the STC tab. Once they are in the “Add Secondary Test Case” function, there are several fields that may be filled in: Secondary Test Case Name; Test Case Condition (e.g., select the disease state grouping that this Secondary Test Case should be tied to); Response Group (e.g., this is the set of options that the pharmacist may be able to choose from during the intervention within the application, for example an intervention focused on administering a flu shot to a patient may use the following options:
Patient accepts—RPh to administer
Patent declines—Has appointment with other provider
Patient declines—Self-administers (if applicable)
Not applicable—Medication picked up by caregiver
Patient declines—Other

An intervention focused on a change in dose recommendation to the prescriber would have different options:
Change in Dose/Form/Interval recommended to Prescriber—Accepted
Change in Dose/Form/Interval recommended to Prescriber—Declined
Change in Dose/Form/Interval recommended to Prescriber—No response or pending
Patient Declined
Educated Patient—No further immediate action required
Not clinically relevant

The super user can select any response group they want. If there is not one that matches what they are trying to do, they can create one.

Directions (e.g., these are instructions to the pharmacist to let them know what the intervention is about, why it is being conducted, what steps may need to be completed, etc.); Provider Recommendation (e.g., this is the prescriber facing language that would be sent if the pharmacist wishes to make a recommendation to them. Examples would be requests for a change in dose, information regarding a drug interaction, or a referral if the patient needs to be seen); Recommendation (e.g., this is the patient friendly language the pharmacist may read to the patient when conducting the intervention); Goal Category 1 & 2 (e.g., these are fields used to map the intervention to SNOMED CT codes for billing purposes); Output record (e.g., allows the user to specify what format the completion data should be logged as); Repeat (e.g., this lets the user determine if the intervention is meant to be a one-time intervention, or if it is acceptable to revisit this topic for the patient. If selected, the user has the option to specify how many days this intervention should be repeated. For example, diabetes patients should have their A1C checked on a quarterly basis); Date Range (e.g., some interventions are only relevant during certain times of the year. For example, flu shots are only given in the fall. This field allows the user to define those limits); and Active (e.g., this is a way to deactivate a Secondary Test Case if needed).

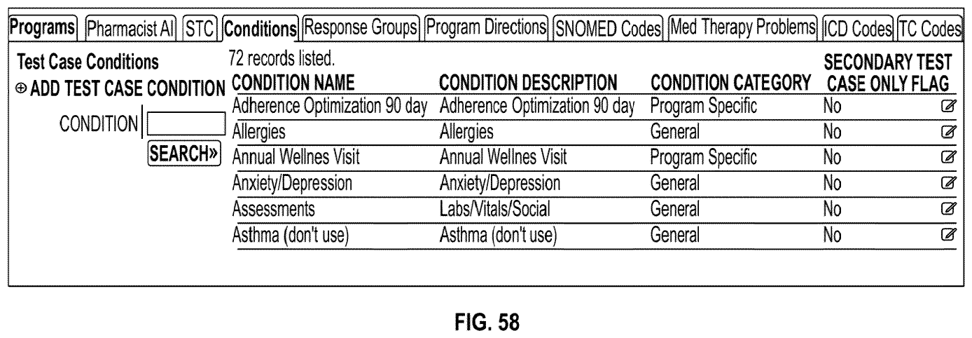

FIG. 58 is an example portion of a graphical user interface showing an example prompt to add a test case condition, in accordance with one or more of the techniques described herein. Additional conditions can be added by clicking the “Add Test Case Condition”. This allows the users to expand the catalog of disease states to address. This can greatly expand the amount of billable interventions the platform can support.

The Condition name is the medical condition. The Description is a short definition of the condition. The Condition Category is used to specify if this condition should be available to be applied to all patients in all programs, or just for a specific program. The Secondary Test Case Only Flag is used to determine if there can be Secondary Test Case Recommendations created using this condition, or it should only be used for an initial intervention.

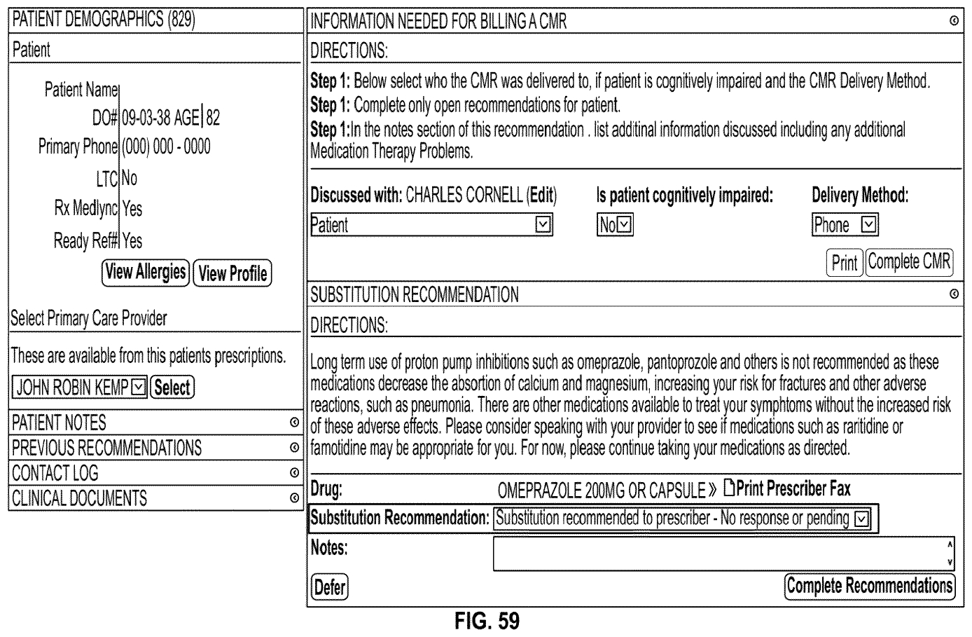

FIG. 59 is an example portion of a graphical user interface showing an example response group option, in accordance with one or more of the techniques described herein. The admin tool allows for the creation of “Response Groups”, which are the set of options that the pharmacist may be able to choose from during the intervention within the application. As new unique interventions are created, it is useful to be able to create new response groups tailored to that recommendation.

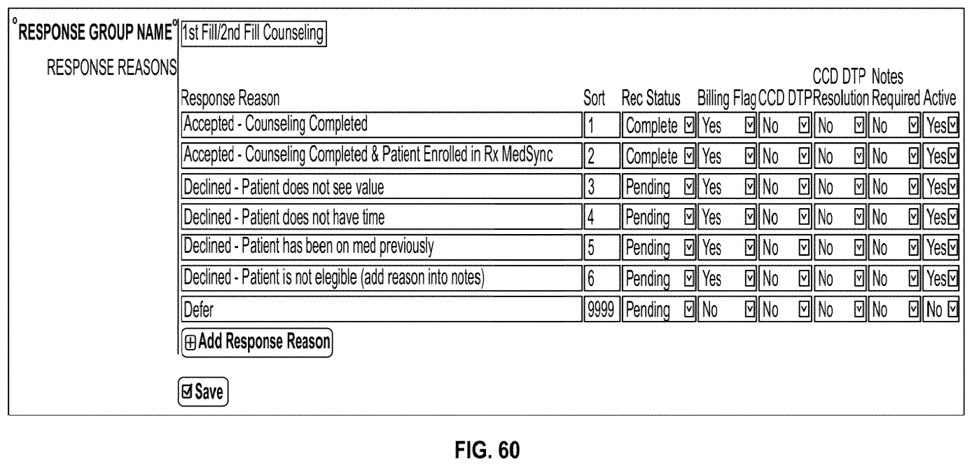

FIG. 60 is an example portion of a graphical user interface showing an example prompt for editing a response group, in accordance with one or more of the techniques described herein. When setting up or editing a response group, the following features are available: Response Group Name (e.g., allows for identification within the list of all response groups. These typically closely match either a SNOMED CT category or a title that refers to a specific program, depending on the application of the response group); Response Reason(s) (e.g., these are the options that the pharmacist can select); Sort (e.g., this is the order the responses may be listed in the drop down for the pharmacist); Rec (Recommendation) Status (e.g., if this response reason is selected, should the system consider this intervention completed. For example, if the system marks a response as “Additional therapy recommendation to prescriber-no response or pending”, then the system should leave the intervention as pending so that the system can go back and change the status if the providers do hear back from the prescriber. This is important because some payers may pay the entity operating these systems if the system gets a response from the prescriber and document that response; Billing Flag: This indicates to the encounter if it is billable; CCD DTP: Flags in the system for the CCD (Continuity of Care Document) if the test case recommendation response is a DTP (Drug Therapy Problem); CCD DTP Resolution: Flags in the system for the CCD if the test case recommendation response is a DTP resolution; Notes Required: enforces the user to enter a note to their response to explain their rationale to their section. The system may still clear that intervention from the active work queue, but the Rec Status would remain pending and the case remains retrievable so the status can be updated); and Filling Flag (e.g., similar to Rec Status, if this option is selected, should the system consider this intervention to be billable. For example, if the option was that the intervention was assessed by the pharmacist to be “not clinically relevant”, then it would not be appropriate to bill the payer in that scenario).

The following documentation describes the underlying logic, configurations and functions of the artificial intelligence model. There is a foundational database, upon which a series of queries are performed to generate the end result of pre-populated interventions for the pharmacists to recommend to their patients. There are two other applications which utilize this database; the web application (Clinical Platform) and the administration application (Clinical Admin Tool). Each of these applications has been designed and built. The Clinical Platform application is a sub application of the application workflow program (under the “Clinical” tab). The web application allows the pharmacist to review all patient recommendations and to complete the recommendations based on their medication profile and pharmacist review. The administration application has two key functions: the ability to administer and create both “Programs” and “Recommendations”.

The program administration allows the system to create a program algorithm (based on specified criteria) over a pool of patients the system would generate recommendations for. For example: an insurance company would like the system to make recommendations for all of their patients that fill medications at pharmacies according to the techniques described herein. Programs criteria include the number of interventions per year, type of recommendations, such as Comprehensive Medication Reviews (CMR) or Follow Up encounters, and how often these can be performed.

The Recommendation administration allows for the creation of interventions based on customizable criteria which are then displayed in the application. Program setup utilizes both Initial and Follow Up encounters as the core intervention types, which are created and displayed.

The artificial intelligence model includes at least five functions: Patient Selection; Script Selection; Pharmacist AI Engine; Secondary Test Case (STC) Condition Engine; and Final Selection.

Patients may be selected to receive an intervention in various ways. A list of patients that are targeted by an outside entity may be furnished to the pharmacies executing the techniques described herein. An example would be an employer group who wanted to have the system screen all of their employees for high blood pressure. They would provide an employee list, and the system would convert it to a flat file and load it into the data warehouse. These patients would then be identified within the application for an intervention.

The artificial intelligence model allows the creation of a “Program” within the administration application which may automatically identify patients that qualify for an intervention based on customizable criteria (e.g. date of birth, age, gender, primary doctor, pharmacy location, nursing home status, and synchronized status, etc.). An example of this would be an insurance company that wants the system to conduct Comprehensive Medication Reviews for all of their patients. The system may look within the database and identify the patients that meet that criteria set. All patient and program combinations are individually evaluated because each program can have a different set of recommendations and conditions tailored to its requirements.

It is possible that one patient may qualify for more than one program at a time. For example, they may be on an insurance plan that wants the system to conduct a CMR, and that patient could be on a drug that is part of a Pharma consultation program. All of the possible interventions may be listed within the patient's profile so the pharmacist can take action on all of the interventions at once.

Once patients have been qualified into their respective program(s), the system loads each patient's scripts for the past 180 days. Key elements of the load include:

NDC (National Drug Code); GCNSEQ (GCN Sequence); TC Code (Therapeutic Code); Quantity; Dose Per Day; Script Number; Fill Number; Store Number; Strength; SIG Code; Refills Allowed; Fill Date; Supply in Days; Current Fill Flag; and Current Fill End Date.

The last two data elements (e.g., Current Fill Flag and Current Fill End Date) are calculated by using the combination of fill date, supply in days, and refills allowed. Current fill flag and current fill end date are used by the analyzer engine to select active scripts to be evaluated by the Pharmacist AI and Secondary Test Case recommendation engines.

The Pharmacist AI recommendation engine is the main function within the artificial intelligence model. This stage is where the selected patient's script data is evaluated against a library of possible recommendations. If a match is found, that recommendation is then displayed in the Clinical Platform for the pharmacist to address with the patient.

There are seven sets of criteria that are evaluated to determine if a recommendation is applicable for a given patient:

Generic Code Number (e.g., all drugs have an NDC number assigned to them. This number represents not only the name of the drug (Lisinopril) and the strength (10 mg), but it also specifies the manufacturer and the pack size for the stock bottle. There could be dozens or hundreds of specific NDCs out there for Lisinopril 10 mg. A GCN attempts to roll all of those individual NDCs into one grouping. Each NDC is tied to one GCN SEQ number. The system typically builds the recommendations at the GCN level. The GCN SEQ based recommendations are based on 2 concepts: ‘Sequence Group’ and ‘Criteria Type’. Sequence Group(s) are the number of groups that belong to the test case for each Criteria Type. For example: A recommendation has 3 sequence groups, each group has 1 or more GCN SEQ numbers in each Sequence Group. This allows the system to write a condition where the system may have a medication in each of the three Sequence Groups above to test positive for this test case. Criteria Type is the type of inclusion or exclusion. In the above example the system may have a medication in all three groups for a positive test case. This Criteria Type would be a ‘select/include’. The second type the system can have would be an ‘exclusion’. Modifying the existing example: to have three ‘select/include’ Sequence groups (like above) and 1 ‘exclude’ Sequence Group. If the system matches all 3 groups above and the system does not have a medication in the exclude criteria this test case would still be positive. If the system had an excluded medication instead then it would invalidate the test case to a negative result. This is a way for the system to write complex GCN SEQ conditions where the system may need to check multiple groups of medications and/or invalidate this condition by one or more medication group exclusions);
Therapeutic Code (TC Code) (e.g., a way of dividing up NDCs based on their ability to treat disease states. Differing from GCN SEQ, an NDC can have multiple TC codes (one for each disease state that can be treated with this medication). TC Codes are treated in the same fashion as GCN SEQ condition above. The system has Sequence Group and Criteria Type that are used in the same way to write complex TC code based conditions for a test case);
Gender (e.g., Gender based recommendation for male or female);
First Fill (e.g., First Fill recommendations occur when the system has a first and or second fill for a particular NDC(s), which is linked to a targeted clinical intervention from an insurance provider or manufacturer. The criteria for first fill can be made at the NDC, GCN SEQ, and or TC code);
Age (e.g., age-based recommendations can be created for specific ranges of ages);
Quantity Dispensed (e.g., recommendations based on the quantity of dosage units dispensed can be created); and
Daily Dose (e.g., dose-based recommendations are identified by looking at the number dosage units prescribed per day and the strength of the medication. To evaluate the strength of the medication a patient is taking per day means that the system may take all the patient's scripts and tally the dose per day they are taking and compare against the range specified in the recommendation).

In relation to Secondary Test Case Engine, the artificial intelligence model may use the medical conditions a patient has to generate intervention opportunities. The system obtains a comprehensive list of conditions in three ways: As prescriptions flow through the application, some of them contain ICD-10 diagnosis codes; the system can infer the medical condition a patient has based on the test case a patient fails; and the system can ask the patient during their initial encounter.

The system can then generate future recommendations for the patient relating to that medical condition, in the form of a Follow Up encounter using the Secondary Test Case (STC) Recommendation Engine. Some programs may require ongoing engagement with a patient after an initial intervention. The STC Engine may determine which STC interventions are queued up for each patient. The cadence of occurrence or reoccurrence is configurable at the program level. Each intervention also has a configurable prioritization to ensure the most relevant and impactful recommendations are completed first.

STC recommendations are triggered off of a patient's disease state assigned via the Pharmacist AI Engine. For example, Pharmacist AI identifies a patient that is on a blood pressure medication. That identification process causes the patient to be tagged as having the disease state of hypertension. Subsequent interventions could then be designed around managing the disease state, such as: Offering a blood pressure screening; conducting a monthly hypertension control assessment; referring the patient to their doctor if their blood pressure is not well controlled; diet and exercise tips; and by creating interventions based on the disease state, rather than just off of the singular blood pressure medication, the system can create a wider array of recommendations.

Additional Follow Ups can also be triggered based on the responses to prior Follow Ups, creating a cascading effect. In the case of the hypertensive patient, in month one, a provider may conduct an initial blood pressure screening, and the system sees that their blood pressure was too high. Based on this, the system would reach out to the doctor to recommend an increase in dose of their medication.

In month two, a new STC generates to conduct a follow up with the patient to see if their doctor has increased their dose. In this case, the answer is yes. The fact that there was a dose change would then trigger another intervention. In month three, the system could recheck their blood pressure and screen for potential side effects. If the pharmacist marks the patient as controlled with no side effects, perhaps no Follow Ups would generate the next month. The next Follow Up that would trigger for this patient could be a quarterly blood pressure re-check.

If the pharmacist marks that the patient is still uncontrolled or experiencing side effects, the system would reach out to the MD again (similarly to the first Follow Up). In this case, the system would get a Follow Up for month four and the sequence would repeat. The end result of the STC Engine is the ability to engage patients longitudinally. This has the effect of improving the patient's health outcomes, while at the same time creating additional reoccurring revenue opportunities for the pharmacy.

For the final selection, once the Pharmacist AI and STC Engines have identified which recommendations are applicable for the patient, the system may assess a patient's history within a given program to ensure that the system only queues up interventions that are billable. For example, if a program only pays for an initial visit with the pharmacist, and that visit occurs in June. In September, a medication is added to the patient's profile that flags in the Pharmacist AI Engine. In this case, the system would not want to conduct a second visit, because it is not billable. The system may exclude the patient from being queued for the pharmacist to intervene with. If the patient qualifies for a different program that is billable, then they would get queued up for a second encounter.

Most clinical platforms do not have access to real time or next day information from a dispensing application. Their data comes from payer data based on pharmacy claims history. This data could be weeks to months old. Real time data allows for immediate generation of interventions, so that those opportunities are not missed when the patient is in the pharmacy.

Another issue with relying on claims data is that it typically does not include the directions. The directions are used to ensure the patient is taking the medications correctly, and also for inclusion on the CMS mandated patient take away documents.

Because this data is absent, the pharmacist has to manually type these in during the medication reconciliation process. This step makes that process take longer, driving inefficiency.

The system collects ICD-10 diagnosis codes as prescriptions are processed through the pharmacist verification process. This makes the system less reliant on inferring diagnosis codes based on the medication the patient is on. This leads to more precise patient records, which leads to more accurate recommendations and increases the number of claims that successfully adjudicate. Pharmacist can add or modify these diagnosis codes during the medication reconciliation process.

The system is unique based on the integration with the dispensing application. The system may output the drug profile, allergies, conditions, etc. within the application. Users do not have to go back into their dispensing application to review that information. Additionally, the integration allows for real time updates to these data sets.

Competitor clinical platforms only show a subset of the patient's medications, based on their most recent claims (typically 30 or 90 days). The system shows older scripts, and the system makes a determination on “active or inactive”. By being able to mark a medication no longer active, the user is able to see past history, however, may know that the medication is no longer applicable. This view help pharmacists make better recommendations. During the medication reconciliation process, the user can toggle the medication status based on their conversation with the patient.

Most integrations between dispensing systems and clinical modules utilize a link out to an external website. The system displays the clinical information directly within the pharmacist's dispensing application. Comparing other clinical management software with the current application, the system is unique because the system includes the pharmacists checking process in the software. This allows for pharmacist workload balancing between pharmacies. Pharmacies with dispensing applications that do not have workload balancing are able to obtain that feature by using the software. That feature improves the pharmacist's ability to complete these interventions.

Clinical interventions are able to be workload balanced across stores, without having to log into each store's portal. Other systems force the user to log in as the other store. The system has a built in credential tracker, ensuring that the cases being presented to the pharmacist can be completed based on the status for their credentialing with the applicable payer.

The system is able to display patients that are eligible for clinical interventions which are hosted in other clinical platforms. This is unique in that the system is able to display all interventions available for a pharmacy to complete in a single application. This makes it easier for the pharmacist to ensure that no interventions are missed, driving revenue and patient care. Additionally, it reduces complexity related to training pharmacists across multiple platforms.

The system simply uses the patient's name, date of birth, and store number, and some details on the nature of the externally hosted intervention. From there, the system can load the opportunity into the dispensing application and have the artificial intelligence model generate interventions for the pharmacist to discuss. Additionally, the system is able to load similar patient data directly from insurance payors or employer groups.

This application is especially unique based on the level of customization available to the super users in the system. Current clinical software suppliers are in full control of the types of interventions available, the verbiage contained within those interventions, etc. Those suppliers act as middlemen between the pharmacies and the payors. The result is that pharmacies are limited in their ability create new revenue streams, based on the book of business the supplier has been able to secure. There could be opportunities that one supplier has, that another does not. In order to go after all possible revenue, a pharmacy would have to subscribe to multiple vendors.

The software cuts out that middleman and puts the pharmacy in control of their book of business. They can work directly with payors, local employers, local health systems, individual doctors; anyone that wants to create a patient care program can be supported on this platform. The pharmacy has all the tools necessary to build their own custom program without having to include a vendor at all. This drastically improves the speed of implementation.

The Clinical Admin Tool is the key to facilitating this flexibility. Super users are able to build customizable programs, recommendations, conditions, and response groups. This allows them to tailor their programs based on the business opportunities they would like to pursue. The system uses custom programs to address: Medication Therapy Management (MTM); Immunization and Medication Administration; Point of Care Testing; DIR Mitigation; Opioid Interventions; Pharma Sponsored Enhanced Counseling Programs; HEDIS Measure Interventions; Chronic Care Management Interventions; and Health Plan, Employer Based Programs, and Department of Health Opportunities-Value Based Care.

These are just the topics that have been chosen to address. There are very likely additional opportunities that other pharmacies would like to pursue, and this system allows them to do so. It is to be recognized that depending on the example, certain acts or events of any of the techniques described herein can be performed in a different sequence, may be added, merged, or left out altogether (e.g., not all described acts or events are necessary for the practice of the techniques). Moreover, in certain examples, acts or events may be performed concurrently, e.g., through multi-threaded processing, interrupt processing, or multiple processors, rather than sequentially.

In one or more examples, the functions described may be implemented in hardware, software, firmware, or any combination thereof. If implemented in software, the functions may be stored on or transmitted over as one or more instructions or code on a computer-readable medium and executed by a hardware-based processing unit. Computer-readable media may include computer-readable storage media, which corresponds to a tangible medium such as data storage media, or communication media including any medium that facilitates transfer of a computer program from one place to another, e.g., according to a communication protocol. In this manner, computer-readable media generally may correspond to (1) tangible computer-readable storage media which is non-transitory or (2) a communication medium such as a signal or carrier wave. Data storage media may be any available media that can be accessed by one or more computers or one or more processors to retrieve instructions, code and/or data structures for implementation of the techniques described in this disclosure. A computer program product may include a computer-readable medium.

By way of example, and not limitation, such computer-readable storage media can comprise RAM, ROM, EEPROM, CD-ROM or other optical disk storage, magnetic disk storage, or other magnetic storage devices, flash memory, or any other medium that can be used to store desired program code in the form of instructions or data structures and that can be accessed by a computer. Also, any connection is properly termed a computer-readable medium. For example, if instructions are transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. It should be understood, however, that computer-readable storage media and data storage media do not include connections, carrier waves, signals, or other transitory media, but are instead directed to non-transitory, tangible storage media. Disk and disc, as used herein, includes compact disc (CD), laser disc, optical disc, digital versatile disc (DVD), floppy disk and Blu-ray disc, where disks usually reproduce data magnetically, while discs reproduce data optically with lasers. Combinations of the above should also be included within the scope of computer-readable media.

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor,” as used herein may refer to any of the foregoing structure or any other structure suitable for implementation of the techniques described herein. In addition, in some aspects, the functionality described herein may be provided within dedicated hardware and/or software modules configured for encoding and decoding, or incorporated in a combined codec. Also, the techniques could be fully implemented in one or more circuits or logic elements.

The techniques of this disclosure may be implemented in a wide variety of devices or apparatuses, including a wireless handset, an integrated circuit (IC) or a set of ICs (e.g., a chip set). Various components, modules, or units are described in this disclosure to emphasize functional aspects of devices configured to perform the disclosed techniques, but do not necessarily require realization by different hardware units. Rather, as described above, various units may be combined in a codec hardware unit or provided by a collection of interoperative hardware units, including one or more processors as described above, in conjunction with suitable software and/or firmware.

Various examples of the disclosure have been described. Any combination of the described systems, operations, or functions is contemplated. These and other examples are within the scope of the following claims.

1. A non-transitory computer-readable storage medium containing instructions that, when executed, cause one or more processors of a computing device to:
create a pharmaceutical profile for a patient, wherein the pharmaceutical profile comprises information regarding one or more of one or more active prescriptions for the patient, one or more inactive past prescriptions for the patient, one or more diagnosis codes for the patient, prescription directions for the one or more active prescriptions for the patient, allergy information for the patient, clinical interventions for the patient, insurance information for the patient, a preferred store for the patient, and demographic information for the patient;
receive real-time prescription information for the patient;
receive a set of criteria for an intervention program set by one of an employer of the patient, an insurance payor of the patient, and a pharmacy for the patient;
determine that a drug identified in the real-time prescription information satisfies a criterion in the set of criteria for the intervention program;
determine, based on the pharmaceutical profile for the patient, that the information in the pharmaceutical profile satisfies a remainder of the criteria in the set of criteria for the intervention program; and
generate, based on the pharmaceutical profile for the patient, the intervention program, and the real-time prescription information for the patient an intervention plan for the patient, wherein the intervention plan comprises specialized instructions for the patient regarding joining the intervention program.

2. The non-transitory computer-readable storage medium of claim 1, wherein the intervention plan comprises one or more actions to be taken by either a pharmacy or the patient when the patient arrives at a pharmacy to pick up a current prescription associated with the real-time prescription information.

3. The non-transitory computer-readable storage medium of claim 2, wherein the one or more actions comprise one or more of:
providing instructions for consuming the current prescription;
providing details regarding how the current prescription integrates with the one or more active prescriptions for the patient;
providing details regarding additional side effects due to the one or more diagnosis codes for the patient;
providing details regarding how the current prescription integrates with the one or more clinical interventions for the patient;
determining a need for a co-pay based on the insurance information for the patient;
providing recommendations based on the one or more inactive prescriptions; and
providing details regarding additional side effects due to the demographic information of the patient.

4. (canceled)

5. (canceled)

6. (canceled)

7. The non-transitory computer-readable storage medium of claim 1, wherein the instructions, when executed, further cause the one or more processors to:
receive a list of one or more past prescriptions for the patient; and
for each of the one or more past prescriptions in the list:
determine whether the respective past prescription has been renewed in the past 30 or 90 days;
responsive to determining that the respective past prescription has been renewed in the past 30 or 90 days, classify the respective prescription as an active prescription in the pharmaceutical profile for the patient; and
responsive to determining that the respective past prescription has not been renewed in the past 30 or 90 days, classify the respective prescription as an inactive prescription in the pharmaceutical profile for the patient.

8. (canceled)

9. (canceled)

10. The non-transitory computer-readable storage medium of claim 1, wherein the instructions, when executed, further cause the one or more processors to:
compare the pharmaceutical profile to respective criteria for each of one or more clinical intervention trials on each of one or more clinical platforms;
determine one or more qualified clinical intervention trials as the one or more clinical intervention trials in the one or more clinical platforms that the patient qualifies for based on the pharmaceutical profile for the patient; and
present the one or more qualified clinical intervention trials and information regarding the one or more qualified clinical intervention trials within a user interface for a dispensing application.

11. The non-transitory computer-readable storage medium of claim 1, wherein the instructions, when executed, further cause the one or more processors to:
receive patient information from one or more of an insurance payor or an employer group; and
generate the pharmaceutical profile for the patient based on the patient information.

12. The non-transitory computer-readable storage medium of claim 1, wherein the pharmaceutical profile comprises one or more of a drug history, a treatment direction history, a drug quantity history, a family history, a laboratory assessment history, a vital assessment history, a social behavior, an allergy history, immunization history, patient height, patient weight, and previous recommendations.

13. The non-transitory computer-readable storage medium of claim 1, wherein the instructions, when executed, further cause the one or more processors to:
schedule, based on the intervention plan, a follow-up appointment for the patient.

14. (canceled)

15. The non-transitory computer-readable storage medium of claim 1, wherein the instructions causing the one or more processors to generate the intervention plan comprise instructions that, when executed, cause the one or more processors to:
determine, based on the pharmaceutical profile for the patient, whether the patient qualifies for particular preventative treatment program, wherein eligibility for the particular preventative treatment program is defined by the patient meeting particular criteria; and
responsive to determining that the patient qualifies for the particular preventative treatment program, include one or more elements of the particular preventative treatment program into the intervention plan.

16. The non-transitory computer-readable storage medium of claim 1, wherein the instructions, when executed, further cause the one or more processors to:
output the intervention plan for review by a medical physician.

17. The non-transitory computer-readable storage medium of claim 1,
wherein the pharmaceutical profile includes at least a list of each medication being taken by the patient, and
wherein the intervention plan comprises an assessment of each medication being taken by the patient and a determination of whether each medication being taken by the patient is appropriate for the patient based on one or more additional characteristics of the patient in the pharmaceutical profile.

18. The non-transitory computer-readable storage medium of claim 1,
wherein the pharmaceutical profile includes information gathered by a pharmacist during an assessment of the patient during a patient encounter, and
wherein the intervention plan comprises a pharmacological treatment recommendation.

19. The non-transitory computer-readable storage medium of claim 1,
wherein the pharmaceutical profile includes information gathered by a pharmacist during an assessment of the patient during a patient encounter, and
wherein the intervention plan comprises a non-pharmacological referral.

20. The non-transitory computer-readable storage medium of claim 1, wherein the instructions causing the one or more processors to generate the intervention plan comprise instructions that, when executed, cause the one or more processors to:
identify, based at least in part on the pharmaceutical profile for the patient, one or more drug therapy problems for one or more of the patient or the pharmacist; and
include the one or more drug therapy problems in the intervention plan.

21. (canceled)

22. A method comprising:
creating, by one or more processors, a pharmaceutical profile for a patient, wherein the pharmaceutical profile comprises information regarding one or more of one or more active prescriptions for the patient, one or more inactive past prescriptions for the patient, one or more diagnosis codes for the patient, prescription directions for the one or more active prescriptions for the patient, allergy information for the patient, clinical interventions for the patient, insurance information for the patient, a preferred store for the patient, and demographic information for the patient;
receiving, by the one or more processors, real-time prescription information for the patient;
receiving, by the one or more processors, a set of criteria for an intervention program set by one of an employer of the patient, an insurance payor of the patient, and a pharmacy for the patient;
determining, by the one or more processors, that a drug identified in the real-time prescription information satisfies a criterion in the set of criteria for the intervention program;
determining, by the one or more processors, based on the pharmaceutical profile for the patient, that the information in the pharmaceutical profile satisfies a remainder of the criteria in the set of criteria for the intervention program; and
generating, by the one or more processors, based on the pharmaceutical profile for the patient, the intervention program, and the real-time prescription information for the patient an intervention plan for the patient, wherein the intervention plan comprises specialized instructions for the patient regarding joining the intervention program.

23. A computing device comprising:
a storage component; and
one or more processors configured to:
create a pharmaceutical profile for a patient, wherein the pharmaceutical profile comprises information regarding one or more of one or more active prescriptions for the patient, one or more inactive past prescriptions for the patient, one or more diagnosis codes for the patient, prescription directions for the one or more active prescriptions for the patient, allergy information for the patient, clinical interventions for the patient, insurance information for the patient, a preferred store for the patient, and demographic information for the patient;
receive real-time prescription information for the patient;
receive a set of criteria for an intervention program set by one of an employer of the patient, an insurance payor of the patient, and a pharmacy for the patient;
determine that a drug identified in the real-time prescription information satisfies a criterion in the set of criteria for the intervention program;
determine, based on the pharmaceutical profile for the patient, that the information in the pharmaceutical profile satisfies a remainder of the criteria in the set of criteria for the intervention program; and
generate, based on the pharmaceutical profile for the patient, the intervention program, and the real-time prescription information for the patient an intervention plan for the patient, wherein the intervention plan comprises specialized instructions for the patient regarding joining the intervention program.

24. The non-transitory computer-readable storage medium of claim 1, wherein the one or more diagnosis codes for the patient are collected as prescriptions for the patient are processed through a pharmacist verification process.

25. The non-transitory computer-readable storage medium of claim 12, wherein the laboratory assessment history or a physiological-sign assessment history comprises blood pressure information.

26. The non-transitory computer-readable storage medium of claim 12, wherein the social behavior comprises one or more of tobacco use, alcohol use, caffeine consumption, and exercise.

27. The non-transitory computer-readable storage medium of claim 13, wherein scheduling the follow-up appointment for the patient comprises using a centralized web-based appointment scheduling tool.

28. The non-transitory computer-readable storage medium of claim 15, wherein the particular criteria comprise one or more of date of birth, age, gender, family doctor, pharmacy location, nursing home status, and synchronized status.

29. The non-transitory computer-readable storage medium of claim 15, wherein the particular preventative treatment program comprises developing or updating a personalized prevention plan.

30. The non-transitory computer-readable storage medium of claim 15, wherein the particular preventative treatment program comprises a checkup selected from the group consisting of an A1c check and a blood pressure check.

31. The non-transitory computer-readable storage medium of claim 16, wherein the review by the medical physician comprises a final sign off for a clinical service.

32. The non-transitory computer-readable storage medium of claim 31, wherein the clinical service is selected from the group consisting of an annual wellness visit, advance care planning, transitional care management, remote patient monitoring, and chronic care management.

33. The non-transitory computer-readable storage medium of claim 17, wherein the determination of whether each medication being taken by the patient is appropriate for the patient comprises determining whether each medication is effective for a medical condition, safe given one or more comorbidities and other medications being taken, and able to be taken by the patient as intended.

34. The non-transitory computer-readable storage medium of claim 20, wherein the one or more drug therapy problems comprise one or more of therapeutic duplication, a drug-disease contraindication, a drug-drug interaction, an incorrect drug dosage, an incorrect duration of drug treatment, a drug-allergy interaction, medication cost, non-adherence, and a gap in therapy comprising a missing medication or immunization.

35. The non-transitory computer-readable storage medium of claim 20, wherein generating the intervention plan further comprises generating patient-facing recommendations to address the one or more drug therapy problems.

36. The non-transitory computer-readable storage medium of claim 35, wherein generating the intervention plan further comprises generating provider-facing pre-written evidence-based communication to address the one or more drug therapy problems with a provider.
